(19) United States
(12) Patent Application Publication
(10) Pub. No.: US 2023/0358751 A1
(43) Pub. Date: Nov. 9, 2023

(54) METHODS FOR THE DETECTION AND TREATMENT OF PROSTATE CANCER

(71) Applicant: Board of Regents, The University of Texas System, Austin, TX (US)

(72) Inventors: Samir HANASH, Houston, TX (US); Jody VYKOUKAL, Houston, TX (US); Timothy THOMPSON, Houston, TX (US); Johannes Francois FAHRMANN, Houston, TX (US)

(21) Appl. No.: 18/145,602

(22) Filed: Dec. 22, 2022

Related U.S. Application Data
(63) Continuation of application No. PCT/US2021/040812, filed on Jul. 8, 2021.
(60) Provisional application No. 63/049,521, filed on Jul. 8, 2020.
(60) Provisional application No. 63/067,601, filed on Aug. 19, 2020.

(51) Int. Cl. G01N 33/574

(52) U.S. Cl. CPC G01N 33/57434; G01N 2405/10; G01N 2800/52; G01N 2800/56; G01N 2800/50

(57) ABSTRACT

Provided are methods and related kits for detection of early stage prostate cancer, and determination of risk of being at risk for progression of prostate cancer.

### CROSS REFERENCE TO RELATED APPLICATIONS

This application is a bypass continuation of International Application No. PCT/US2021/040812, filed Jul. 8, 2021, which claims the benefit of U.S. Provisional Patent Application No. 63/049,521, filed Jul. 8, 2020, and U.S. Provisional Patent Application No. 63/067,601, filed Aug. 19, 2020, the disclosures of which are incorporated by reference herein in their entireties.

The patent or application file contains at least one drawing executed in color. Copies of this patent or patent application publication with color drawing(s) will be provided by the Office upon request and payment of the necessary fee.

### STATEMENT REGARDING FEDERALLY SPONSORED RESEARCH

This invention was made with government support under grant number CA223527 awarded by the National Institutes of Health. The government has certain rights in the invention.

### BACKGROUND

Elevated serum Cav-1 levels have been associated with high-risk prostate cancer, castration-resistance, and biochemical recurrence after prostatectomy. Previously it has been demonstrated that increased plasma Cav-1 are associated with early disease reclassification in individuals with prostate cancer that initially present with clinically localized disease. Cav-1 is the eponymous protein component of caveolae: bulb-shaped, 50-100 nm invaginations of the plasma membrane that are enriched in glycosphingolipids and cholesterol. Cav-1 also functions in organizing membrane microdomain composition and in modulating transmembrane signal transduction. Increasing evidence indicates that Cav-1 functions as an essential lipid chaperone to facilitate cellular lipid trafficking and homeostasis, endo- and exocytosis, and mechanoprotection of cell membranes. Cav-1 is known to transport molecules including insulin, chemokines, albumin and low- and high-density lipoproteins (LDL and HDL). Recently, Cav-1 containing extracellular vesicles in white adipose tissue were found to traffic intracellular exchange of protein and lipid between endothelial cells and adipocytes in response to system metabolic state.

In cancer, the role of Cav-1 is dynamic and context-dependent. Cav-1 has been demonstrated to regulate and promote the activities of receptor tyrosine kinases, G-protein coupled receptors, integrins and cadherins. Expression of Cav-1 has been closely associated with aggressive phenotypes in various tumor types and has been linked to epithelial-mesenchymal plasticity, tumor invasion and metastatic potential, and radio- and multidrug resistance.

Although Cav-1 has been associated with altered metabolism in prostate cancer, the mechanism by which Cav-1 effects metabolic rewiring has not been previously determined. Interrogation of Cav-1 function in the context of prostate tumor metabolism uncovered an integrated metabolic program of enhanced lipid scavenging and differential ceramide metabolism active in prostate tumors that exhibit Gleason grade progression following initial enrollment into active surveillance. Importantly, this metabolic phenotype yields biomarkers of disease progression and identifies points of therapeutic susceptibility. Key features of this tumor supportive metabolic program include Cav-1 mediated lipid uptake, increased tumor catabolism of extracellular sphingomyelins (SMs), and altered ceramide metabolism coupled with increased glycosphingolipid synthesis and efflux of circulating Cav-1-sphingolipid particles that comprise cargoes indicative of intersection with mitochondrial remodeling. On the basis of these mechanistic findings, potential actionable metabolic vulnerabilities by targeting Cav-1-mediated lipid scavenging and metabolism in a mouse model of aggressive prostate cancer have been tested.

Metabolomic profiling of baseline plasmas from a longitudinal prospective cohort of prostate cancer active surveillance (AS) participants identified alterations in plasma sphingolipids as prominent features in AS progressor subjects. These metabolite features can be combined to yield a signature predictive for disease progression in early-stage prostate cancer. Previous work showed that baseline plasma caveolin-1 (Cav-1) was an independent predictor of disease classification in a similar AS cohort. This study was predicated on the well-established role of tissue localized and secreted Cav-1 in aggressive, and potentially drug-resistant prostate cancer. Plasma Cav-1 can additionally be integrated with plasma sphingolipid features into a combined predictive signature. Mechanistic studies have been performed to explicate principal biological processes involved in the tumor supportive onco-metabolism underlying the observed plasma signature features. Using a syngeneic RM-9 mouse model of prostate cancer and established human prostate cancer cell lines, evidence has been obtained that Cav-1 promotes rewiring of cancer cell lipid metabolism towards a program of exogenous lipid scavenging and vesicle biogenesis intersecting with sphingolipid metabolism; that activation of this program is evidenced in a plasma signature; and that this program presents a metabolic vulnerability that is targetable as anti-tumor therapy.

### SUMMARY

Provided are methods and kits for evaluating the status of prostate cancer. The methods and kits use multiple assays of biomarkers contained within a biological sample obtained from a subject. The analysis of one or more of the biomarkers: CAV-1, SM(40:2), SM(44:2), lactosylceramide(32:0) (“LacCer 32:0”), lactosylceramide(36:0) (“LacCer 36:0”), trihexosylceramide(34:1) (“TriHexCer”) and hexosylceramide(40:0) (“HexCer 40:0”), provides high-accuracy risk assessment and prognosis for the progression of prostate cancer.

A regression model was identified that can predict the risk of prostate cancer progression for a subject based on the levels of one or more of the biomarkers CAV-1, SM(40:2), SM(44:2), LacCer 32:0, LacCer 36:0, TriHexCer 34:1 and HexCer 40:0 that are found in a biological sample from the subject.

Accordingly, provided herein are methods of determining and/or quantifying the risk for the progression of pancreatic cancer in a subject, comprising measuring the level of one or more of the biomarkers CAV-1, SM(40:2), SM(44:2), LacCer 32:0, LacCer 36:0, TriHexCer 34:1 and HexCer 40:0, in a sample from the subject.

Also provided are methods of treatment or prevention of progression of prostate cancer in a subject in whom the levels of one or more of the biomarkers CAV-1, SM(40:2), SM(44:2), LacCer 32:0, LacCer 36:0, TriHexCer 34:1 and HexCer 40:0, classifies the subject as being at risk for progression of prostate cancer.

Also provided are corresponding kits for determining the presence of indicators for progression of prostate cancer in a sample from the subject, for determening the risk for progression of prostate cancer subject, and for determining and/or quantifying the risk for the progression of prostate cancer in a subject, comprising materials for measuring one or more of the biomarkers CAV-1, SM(40:2), SM(44:2), LacCer 32:0, LacCer 36:0, TriHexCer 34:1 and HexCer 40:0 in the sample.

In some embodiments, biomarkers are measured in blood samples drawn from subjects. In some embodiments, the presence or absence or, alternatively, the quantity, of biomarkers in a biological sample can be determined. In some embodiments, the level of biomarkers in a biological sample can be quantified.

In some embodiments, a surface is provided to analyze a biological sample. In some embodiment, biomarkers of interest adsorb nonspecifically onto this surface. In some embodiments, receptors specific for biomarkers of interest are incorporated onto this surface. In some embodiments, the surface is associated with a particle, for example, a bead.

In some embodiments, the biomarker binds to a particular receptor molecule, and the presence or absence or, alternatively, the quantity, of the biomarker-receptor complex can be determined. In some embodiments, the amount of biomarker-receptor complex can be quantified. In some embodiments, the receptor molecule is linked to an enzyme to facilitate detection and quantification.

In some embodiments, the biomarker binds to a particular relay molecule, and the biomarker-relay molecule complex in turn binds to a receptor molecule. In some embodiments, the presence or absence or, alternatively, the quantity, of the biomarker-relay-receptor complex can be determined. In some embodiments, the amount of biomarker-relay-receptor complex can be quantified. In some embodiments, the receptor molecule is linked to an enzyme to facilitate detection and quantification.

In some embodiments, a biological sample is analyzed sequentially for individual biomarkers. In some embodiments, a biological sample is divided into separate portions to allow for simultaneous analysis for multiple biomarkers. In some embodiments, a biological sample is analyzed in a single process for multiple biomarkers.

In some embodiments, the absence or presence of biomarker can be determined by visual inspection. In some embodiments, the quantity of biomarker can be determined by use of a spectroscopic technique. In some embodiments, the spectroscopic technique is mass spectrometry. In some embodiments, the spectroscopic technique is UV/Vis spectrometry. In some embodiments, the spectroscopic technique is an excitation/emission technique such as fluorescence spectrometry. In some embodiments, the spectroscopic technique is mass spectrometry. In some embodiments, the spectroscopic technique is combined with a chromatographic technique. In some embodiments, the chromatographic technique is liquid chromatography. In some embodiments, the chromatographic technique is high-performance liquid chromatography (“HPLC”). In some embodiments, the chromatographic technique is gas chromatography (“GC”).

In some embodiments, the analysis of biomarkers CAV-1, SM(40:2), SM(44:2), LacCer 32:0, LacCer 36:0, TriHexCer 34:1 and HexCer 40:0 can be combined with analysis of additional biomarkers. In some embodiments, the additional biomarkers can be protein biomarkers. In some embodiments, the additional biomarkers can be non-protein biomarkers.

In some embodiments, a kit is provided for analysis of a biological sample. In some embodiments, the kit can contain the chemicals and reagents required to perform the analysis. In some embodiments, the kit contains a means for manipulating biological samples in order to minimize the required operator intervention. In some embodiments, the kit can record the outcome of an analysis digitally. In some embodiments, the kit can perform any needed mathematical processing of data generated by the analysis.

In another aspect, the disclosure provides a method of determining the risk of progression for prostate cancer in a subject, utilizing a biomarker panel and a protein marker panel wherein the biomarker panel comprises one or more of the biomarkers CAV-1, SM(40:2), SM(44:2), LacCer 32:0, LacCer 36:0, TriHexCer 34:1 and HexCer 40:0; wherein the method comprises performing the following steps on a biological sample obtained from the subject; measuring the levels of the biomarkers and the protein biomarkers in the biological sample; wherein the amount of the biomarkers and the protein biomarkers determines the risk of progression of prostate cancer in the subject.

In another aspect, the disclosure provides a kit for a method as described herein, comprising a first reagent solution that comprises a first solute for detection of CAV-1, a second reagent solution that comprises a second solute for detection of SM(40:2), a third reagent solution that comprises a third solute for detection of SM(44:2), a fourth reagent solution that comprises a fourth solute for detection of LacCer 32:0, a fifth reagent solution that comprises a fifth solute for detection of LacCer 36:0, a sixth reagent solution that comprises a sixth solute for detection of TriHexCer 34:1, and a seventh reagent solution that comprises a seventh solute for detection of HexCer 40:0.

In one embodiment, such a kit comprises a device for contacting the reagent solutions with a biological sample. In another embodiment, such a kit comprises at least one surface with means for binding at least one biomarker. In another embodiment, the at least one biomarker is selected from the group consisting of CAV-1, SM(40:2), SM(44:2), LacCer 32:0, LacCer 36:0, TriHexCer 34:1 and HexCer 40:0.

In another aspect, the disclosure provides a method of treating a subject suspected of risk for progression of prostate cancer, comprising: analyzing the subject for risk of progression of prostate cancer with a method as described herein and administering a therapeutically effective amount of a treatment for the prostate cancer. In one embodiment, the treatment is surgery, chemotherapy, radiation therapy, targeted therapy, or a combination thereof. In another embodiment, such a method comprises at least one receptor molecule that selectively binds to one or more of the biomarkers selected from the group consisting of CAV-1, SM(40:2), SM(44:2), LacCer 32:0, LacCer 36:0, TriHexCer 34:1 and HexCer 40:0. In another embodiment, detection of the amount of CAV-1, SM(40:2), SM(44:2), LacCer 32:0, LacCer 36:0, TriHexCer 34:1 and HexCer 40:0 comprises the use of a solid particle. In another embodiment, the solid particle is a bead. In another embodiment, at least one of the reporter molecules is linked to an enzyme. In another embodiment, at least one of the protein or metabolite markers generates a detectable signal. In another embodiment, the detectable signal is detectable by a spectrometric method. In another embodiment, the spectrometric method is mass spectrometry. In another embodiment, such a method comprises inclusion of patient history information into the assignment of being at risk for progression of prostate cancer or not being at risk for progression cancer. In another embodiment, such a method comprises administering at least one alternate diagnostic test for a patient assigned as being at risk for progression of prostate cancer.

In another aspect, the disclosure provides a method of treatment or prevention of progression of prostate cancer in a subject in whom the levels of one or more of the biomarkers CAV-1, SM(40:2), SM(44:2), LacCer 32:0, LacCer 36:0, TriHexCer 34:1 and HexCer 40:0 identifies a risk for progression of prostate cancer in the subject, comprising one or more steps of: administering a chemotherapeutic drug to the subject with prostate cancer; administering therapeutic radiation to the subject with prostate cancer; and surgery for partial or complete surgical removal of cancerous tissue in the subject with prostate cancer. In one embodiment, the levels of CAV-1, SM(40:2), SM(44:2), LacCer 32:0, LacCer 36:0, TriHexCer 34:1 and HexCer 40:0 are elevated. In another embodiment, the levels of CAV-1, SM(40:2), SM(44:2), LacCer 32:0, LacCer 36:0, TriHexCer 34:1 and HexCer 40:0 are elevated in comparison to the levels of CAV-1, SM(40:2), SM(44:2), LacCer 32:0, LacCer 36:0, TriHexCer 34:1 and HexCer 40:0 in a reference subject or group that is not at risk for progression of prostate cancer. In another embodiment, the reference subject or group is healthy. In another embodiment, the reference subject or group has indolent prostate cancer. In another embodiment, the levels of TriHexCer 34:1 and SM 40:2 are elevated in the subject relative to a healthy subject. In another embodiment, the levels of TriHexCer 34:1 and SM 40:2 are elevated in comparison to the levels of in a reference subject or group that does not have aggressive prostate cancer. In another embodiment, the levels of TriHexCer 34:1 and SM 40:2 are elevated in comparison to the levels of in a reference subject or group that has indolent prostate cancer.

In another aspect, the disclosure provides a method of treatment or prevention of progression of prostate cancer in a subject in whom the levels of CAV-1, SM(40:2), SM(44:2), LacCer 32:0, LacCer 36:0, TriHexCer 34:1 and HexCer 40:0 identifies the subject as having or being at risk for progression of prostate cancer comprising one or more of: administering a chemotherapeutic drug to the subject with prostate cancer; administering therapeutic radiation to the subject with prostate cancer; and surgery for partial or complete surgical removal of cancerous tissue in the subject with prostate cancer. In one embodiment, the levels of CAV-1, SM(40:2), SM(44:2), LacCer 32:0, LacCer 36:0, TriHexCer 34:1 and HexCer 40:0 are elevated. In another embodiment, the levels of CAV-1, SM(40:2), SM(44:2), LacCer 32:0, LacCer 36:0, TriHexCer 34:1 and HexCer 40:0 are elevated in comparison to the levels of CAV-1, SM(40:2), SM(44:2), LacCer 32:0, LacCer 36:0, TriHexCer 34:1 and HexCer 40:0 in a reference subject or group that is not at risk for progression of prostate cancer. In another embodiment, the reference subject or group is healthy. In another embodiment, the reference subject or group has indolent prostate cancer. In another embodiment, the levels of CAV-1, SM(40:2), SM(44:2), LacCer 32:0, LacCer 36:0, TriHexCer 34:1 and HexCer 40:0 are elevated in comparison to the levels of CAV-1, SM(40:2), SM(44:2), LacCer 32:0, LacCer 36:0, TriHexCer 34:1 and HexCer 40:0 in a reference subject or group that has adenocarcinoma. In another embodiment, the levels of CAV-1, SM(40:2), SM(44:2), LacCer 32:0, LacCer 36:0, TriHexCer 34:1 and HexCer 40:0 are elevated in comparison to the levels of CAV-1, SM(40:2), SM(44:2), LacCer 32:0, LacCer 36:0, TriHexCer 34:1 and HexCer 40:0 in a reference subject or group that has squamous cell cancer. In another embodiment, the subject is at high risk for progression of prostate cancer.

In another aspect, the disclosure provides a method of treating a subject suspected of risk for progression of prostate cancer, comprising analyzing the subject for risk for progression of prostate cancer with a method as disclosed herein; administering a therapeutically effective amount of a treatment for the prostate cancer. In one embodiment, the treatment is surgery, chemotherapy, radiation therapy, targeted therapy, or a combination thereof.

Also provided is a method of classifying a subject with prostate cancer as being at risk of developing aggressive prostate cancer or not being at risk of developing aggressive prostate cancer, predicting a predisposition to aggressive prostate cancer in a subject, diagnosing aggressive prostate cancer in a subject with prostate cancer, determining the risk of a subject for having aggressive prostate cancer, predicting the likelihood of progression of prostate cancer in a subject with prostate cancer, providing a prognosis for a subject with prostate cancer, or selecting a subject with prostate cancer for treatment with an anticancer therapy, comprising: (a) measuring the levels of one or more of the biomarkers Caveolin-1 (CAV-1), sphingomyelin 40:2 (SM 40:2), sphingomyelin 44:2 (SM 44:2), lactosylceramide 32:0 (LacCer 32:0), lactosylceramide 36:0 (LacCer 36:0), trihexosylceramide 34:1 (TriHexCer 34:1), and hexosylceramide 40:0 (HexCer 40:0) in a biological sample from said subject using an in vitro assay and (b) comparing the levels of one or more of the biomarkers CAV-1, SM 40:2, SM 44:2, LacCer 32:0, LacCer 36:0, TriHexCer 34:1, and HexCer 40:0 in said sample with a reference, wherein an altered amount of one or more of the biomarkers CAV-1, SM 40:2, SM 44:2, LacCer 32:0, LacCer 36:0, TriHexCer 34:1, and HexCer 40:0 relative to said reference provides an indication selected from the group consisting of: an indication that the subject is at risk of developing aggressive prostate cancer or not at risk of developing aggressive prostate cancer, an indication of a predisposition of the subject to aggressive prostate cancer, an indication of the likelihood of progression of the prostate cancer in the subject, an indication of progression-free survival of the subject, an indication of the likely outcome of treatment of the prostate cancer, and an indication that the subject is a candidate for treatment with an anticancer therapy.

Also provided is a method of classifying a subject with prostate cancer as being at risk of developing aggressive prostate cancer or not being at risk of developing aggressive prostate cancer, predicting a predisposition to aggressive prostate cancer in a subject, diagnosing aggressive prostate cancer in a subject with prostate cancer, determining the risk of a subject for having aggressive prostate cancer, predicting the likelihood of progression of prostate cancer in a subject with prostate cancer, providing a prognosis for a subject with prostate cancer, or selecting a subject with prostate cancer for treatment with an anticancer therapy, comprising: (a) measuring the levels of sphingomyelin 40:2 (SM 40:2), sphingomyelin 44:2 (SM 44:2), lactosylceramide 32:0 (LacCer 32:0), lactosylceramide 36:0 (LacCer 36:0), and trihexosylceramide 34:1 (TriHexCer 34:1) in a biological sample from said subject using an in vitro assay and (b) comparing the levels of SM 40:2, SM 44:2, LacCer 32:0, LacCer 36:0, and TriHexCer 34:1 in said sample with a reference, wherein an altered amount of SM 40:2, SM 44:2, LacCer 32:0, LacCer 36:0, and TriHexCer 34:1 relative to said reference provides an indication selected from the group consisting of: an indication that the subject is at risk of developing aggressive prostate cancer or not at risk of developing aggressive prostate cancer, an indication of a predisposition of the subject to aggressive prostate cancer, an indication of the likelihood of progression of the prostate cancer in the subject, an indication of progression-free survival of the subject, an indication of the likely outcome of treatment of the prostate cancer, and an indication that the subject is a candidate for treatment with an anticancer therapy.

Also provided is a method of classifying a subject with prostate cancer as being at risk of developing aggressive prostate cancer or not being at risk of developing aggressive prostate cancer, predicting a predisposition to aggressive prostate cancer in a subject, diagnosing aggressive prostate cancer in a subject with prostate cancer, determining the risk of a subject for having aggressive prostate cancer, predicting the likelihood of progression of prostate cancer in a subject with prostate cancer, providing a prognosis for a subject with prostate cancer, or selecting a subject with prostate cancer for treatment with an anticancer therapy, comprising: (a) measuring the levels of sphingomyelin 40:2 (SM 40:2), lactosylceramide 36:0 (LacCer 36:0), and trihexosylceramide 34:1 (TriHexCer 34:1) in a biological sample from said subject using an in vitro assay and (b) comparing the levels of SM 40:2, LacCer 36:0, and TriHexCer 34:1 in said sample with a reference, wherein an altered amount of SM 40:2, LacCer 36:0, and TriHexCer 34:1 relative to said reference provides an indication selected from the group consisting of an indication that the subject is at risk of developing aggressive prostate cancer or not at risk of developing aggressive prostate cancer, an indication of a predisposition of the subject to aggressive prostate cancer, an indication of the likelihood of progression of the prostate cancer in the subject, an indication of progression-free survival of the subject, an indication of the likely outcome of treatment of the prostate cancer, and an indication that the subject is a candidate for treatment with an anticancer therapy.

Also provided is a method of classifying a subject with prostate cancer as being at risk of developing aggressive prostate cancer or not being at risk of developing aggressive prostate cancer, predicting a predisposition to aggressive prostate cancer in a subject, diagnosing aggressive prostate cancer in a subject with prostate cancer, determining the risk of a subject for having aggressive prostate cancer, predicting the likelihood of progression of prostate cancer in a subject with prostate cancer, providing a prognosis for a subject with prostate cancer, or selecting a subject with prostate cancer for treatment with an anticancer therapy, comprising (a) measuring the levels of trihexosylceramide 34:1 (TriHexCer 34:1) in a biological sample from said subject using an in vitro assay and (b) comparing the levels of TriHexCer 34:1 in said sample with a reference, wherein an altered amount of TriHexCer 34:1 relative to said reference provides an indication selected from the group consisting of an indication that the subject is at risk of developing aggressive prostate cancer or not at risk of developing aggressive prostate cancer, an indication of a predisposition of the subject to aggressive prostate cancer, an indication of the likelihood of progression of the prostate cancer in the subject, an indication of progression-free survival of the subject, an indication of the likely outcome of treatment of the prostate cancer, and an indication that the subject is a candidate for treatment with an anticancer therapy.

Also provided is a method of classifying a subject with prostate cancer as being at risk of developing aggressive prostate cancer or not being at risk of developing aggressive prostate cancer, predicting a predisposition to aggressive prostate cancer in a subject, diagnosing aggressive prostate cancer in a subject with prostate cancer, determining the risk of a subject for having aggressive prostate cancer, predicting the likelihood of progression of prostate cancer in a subject with prostate cancer, providing a prognosis for a subject with prostate cancer, or selecting a subject with prostate cancer for treatment with an anticancer therapy, comprising (a) measuring the levels of sphingomyelin 40:2 (SM 40:2) in a biological sample from said subject using an in vitro assay and (b) comparing the levels of SM 40:2 in said sample with a reference, wherein an altered amount of SM 40:2 relative to said reference provides an indication selected from the group consisting of an indication that the subject is at risk of developing aggressive prostate cancer or not at risk of developing aggressive prostate cancer, an indication of a predisposition of the subject to aggressive prostate cancer, an indication of the likelihood of progression of the prostate cancer in the subject, an indication of progression-free survival of the subject, an indication of the likely outcome of treatment of the prostate cancer, and an indication that the subject is a candidate for treatment with an anticancer therapy.

Also provided is a method of classifying a subject with prostate cancer as being at risk of developing aggressive prostate cancer or not being at risk of developing aggressive prostate cancer, predicting a predisposition to aggressive prostate cancer in a subject, diagnosing aggressive prostate cancer in a subject with prostate cancer, determining the risk of a subject for having aggressive prostate cancer, predicting the likelihood of progression of prostate cancer in a subject with prostate cancer, providing a prognosis for a subject with prostate cancer, or selecting a subject with prostate cancer for treatment with an anticancer therapy, comprising (a) measuring the levels of: sphingomyelin 40:2 (SM 40:2), lactosylceramide 36:0 (LacCer 36:0), and trihexosylceramide 34:1; and/or trihexosylceramide 34:1; and/or sphingomyelin 40:2 (SM 40:2) in a biological sample from said subject using an in vitro assay and (b) comparing the levels of SM 40:2 in said sample with a reference, wherein an altered amount of SM 40:2 relative to said reference provides an indication selected from the group consisting of an indication that the subject is at risk of developing aggressive prostate cancer or not at risk of developing aggressive prostate cancer, an indication of a predisposition of the subject to aggressive prostate cancer, an indication of the likelihood of progression of the prostate cancer in the subject, an indication of progression-free survival of the subject, an indication of the likely outcome of treatment of the prostate cancer, and an indication that the subject is a candidate for treatment with an anticancer therapy.

Also provided is a method of treatment or prevention of progression of prostate cancer in a subject in whom the levels of sphingomyelin 40:2 (SM 40:2), lactosylceramide 36:0 (LacCer 36:0), and trihexosylceramide 34:1 are elevated relative to a reference without prostate cancer, comprising one or more of: administering an anticancer drug to the subject with prostate cancer; administering therapeutic radiation to the subject with prostate cancer; and surgery for partial or complete surgical removal of cancerous tissue in the subject with prostate cancer.

Also provided is a method of treatment or prevention of progression of prostate cancer in a subject in whom the levels of trihexosylceramide 34:1 are elevated relative to a reference without prostate cancer, comprising one or more of: administering an anticancer drug to the subject with prostate cancer; administering therapeutic radiation to the subject with prostate cancer; and surgery for partial or complete surgical removal of cancerous tissue in the subject with prostate cancer.

Also provided is a method of treatment or prevention of progression of prostate cancer in a subject in whom the levels of sphingomyelin 40:2 (SM 40:2) are elevated relative to a reference without prostate cancer, comprising one or more of: administering an anticancer drug to the subject with prostate cancer; administering therapeutic radiation to the subject with prostate cancer; and surgery for partial or complete surgical removal of cancerous tissue in the subject with prostate cancer.

Also provided is a method of treatment or prevention of progression of prostate cancer in a subject in whom the levels of: (a) sphingomyelin 40:2 (SM 40:2), lactosylceramide 36:0 (LacCer 36:0), and trihexosylceramide 34:1; and/or (b) trihexosylceramide 34:1; and/or (c) sphingomyelin 40:2 (SM 40:2) are elevated relative to a reference without prostate cancer, comprising one or more of: administering an anticancer drug to the subject with prostate cancer; administering therapeutic radiation to the subject with prostate cancer; and surgery for partial or complete surgical removal of cancerous tissue in the subject with prostate cancer.

Also provided is a diagnostic panel for aggressive prostate cancer comprising Caveolin-1 (CAV-1), sphingomyelin 40:2 (SM 40:2), sphingomyelin 44:2 (SM 44:2), lactosylceramide 32:0 (LacCer 32:0), lactosylceramide 36:0 (LacCer 36:0), trihexosylceramide 34:1 (TriHexCer 34:1), and hexosylceramide 40:0 (HexCer 40:0).

Also provided is a diagnostic panel for aggressive prostate cancer comprising sphingomyelin 40:2 (SM 40:2), sphingomyelin 44:2 (SM 44:2), lactosylceramide 32:0 (LacCer 32:0), lactosylceramide 36:0 (LacCer 36:0), and trihexosylceramide 34:1 (TriHexCer 34:1).

Also provided is a diagnostic panel for aggressive prostate cancer comprising sphingomyelin 40:2 (SM 40:2), lactosylceramide 36:0 (LacCer 36:0), and trihexosylceramide 34:1 (TriHexCer 34:1).

Also provided is a diagnostic panel for aggressive prostate cancer comprising trihexosylceramide 34:1 (TriHexCer 34:1).

Also provided is a diagnostic panel for aggressive prostate cancer comprising sphingomyelin 40:2 (SM 40:2).

Also provided is a diagnostic panel for aggressive prostate cancer comprising: sphingomyelin 40:2 (SM 40:2), lactosylceramide 36:0 (LacCer 36:0), and trihexosylceramide 34:1; and/or trihexosylceramide 34:1; and/or sphingomyelin 40:2 (SM 40:2).

Also provided is a method of treatment or prevention of progression of prostate cancer in a subject in whom the levels of Caveolin-1 (CAV-1), sphingomyelin 40:2 (SM 40:2), sphingomyelin 44:2 (SM 44:2), lactosylceramide 32:0 (LacCer 32:0), lactosylceramide 36:0 (LacCer 36:0), trihexosylceramide 34:1 and hexosylceramide 40:0 are elevated relative to a reference without prostate cancer, comprising one or more of administering an anticancer drug to the subject with prostate cancer; administering therapeutic radiation to the subject with prostate cancer; and surgery for partial or complete surgical removal of cancerous tissue in the subject with prostate cancer.

Also provided is a method of treatment or prevention of progression of prostate cancer in a subject in whom the levels of sphingomyelin 40:2 (SM 40:2), sphingomyelin 44:2 (SM 44:2), lactosylceramide 32:0 (LacCer 32:0), lactosylceramide 36:0 (LacCer 36:0), and trihexosylceramide 34:1 are elevated relative to a reference without prostate cancer, comprising one or more of administering an anticancer drug to the subject with prostate cancer; administering therapeutic radiation to the subject with prostate cancer; and surgery for partial or complete surgical removal of cancerous tissue in the subject with prostate cancer.

Also provided is a method of treatment or prevention of progression of prostate cancer in a subject in whom the levels of sphingomyelin 40:2 (SM 40:2), lactosylceramide 36:0 (LacCer 36:0), and trihexosylceramide 34:1 are elevated relative to a reference without prostate cancer, comprising one or more of administering an anticancer drug to the subject with prostate cancer; administering therapeutic radiation to the subject with prostate cancer; and surgery for partial or complete surgical removal of cancerous tissue in the subject with prostate cancer.

Also provided is a method of treatment or prevention of progression of prostate cancer in a subject in whom the levels of trihexosylceramide 34:1 are elevated relative to a reference without prostate cancer, comprising one or more of administering an anticancer drug to the subject with prostate cancer; administering therapeutic radiation to the subject with prostate cancer; and surgery for partial or complete surgical removal of cancerous tissue in the subject with prostate cancer.

Also provided is a method of treatment or prevention of progression of prostate cancer in a subject in whom the levels of sphingomyelin 40:2 (SM 40:2) are elevated relative to a reference without prostate cancer, comprising one or more of administering an anticancer drug to the subject with prostate cancer; administering therapeutic radiation to the subject with prostate cancer; and surgery for partial or complete surgical removal of cancerous tissue in the subject with prostate cancer.

Also provided is a method of treatment or prevention of progression of prostate cancer in a subject in whom the levels of sphingomyelin 40:2 (SM 40:2), lactosylceramide 36:0 (LacCer 36:0), and trihexosylceramide 34:1; and/or trihexosylceramide 34:1; and/or sphingomyelin 40:2 (SM 40:2) are elevated relative to a reference without prostate cancer, comprising one or more of administering an anticancer drug to the subject with prostate cancer; administering therapeutic radiation to the subject with prostate cancer; and surgery for partial or complete surgical removal of cancerous tissue in the subject with prostate cancer.

### BRIEF DESCRIPTION OF THE DRAWINGS

FIG. 1A depicts individual ROC AUC for plasmosphingomyelins and glycosphingolipids (light grey=baseline; dark grey=12 month) in early stage prostate cancer patients experiencing disease progression. (i) SM(32:1) (ii) SM(32:2) (ii) SM(34:1) (iv) SM(34:2) (v) SM(36:1) (vi) SM(36:2) (vii) SM(40:2) (viii) SM(42:1) (ix) SM(42:3) (x) GlucosylCer(39:2) (xi) LactosylCer(32:0) (xii) LactosylCer(32:1) (xiii) LactosylCer(34:1) (xiv) TrihexosylCer(d18:1/16:0) (xv) DihexosylCer(34:1) (xvi) DihexosylCer(36:1) (xvii) TrihexosylCer(34:1). FIG. 1B depicts Volcano plot illustrating hazard ratios (horizontal) for individual plasma lipid species stratified by lipid domains in predicting disease progression using baseline plasma samples from the larger prospective cohort (n=459); vertical axis=−log(p-value). FIG. 1C depicts Kaplan-Meier survival curve illustrates progression free survival (vertical axis) over time (months; horizontal axis) for participants with a plasma sphingolipid signature (plasma Cav-1 plus 6 sphingolipids); plasma sphingolipid signature levels≤4.33 or >4.33.

FIGS. 2A-2Q depict intra-patient comparison of sphingolipids identified in the Discovery Cohort. (i) aggressive_baseline; (ii) aggressive_12M; (2A) glucosylceramide (38:2) (2B) lactosylceramide (32:0) (2C) lactosylceramide (32:1) (2D) lactosylceramide (34:1) (2E) NeuAc?2-3Gal?1-4Glc?-Cer(d18:1/16:0) (2F) NeuAc?2-3Gal?-Cer(34:1) (2G) NeuAc?2-3Gal?-Cer(36:1) (2H) TriHexCer 34:1 (2I) SM(32:1) (2J) SM(32:2) (2K) SM(34:1) (2L) SM(34:2) (2M) SM(36:1) (2N) SM(36:2) (2O) SM(40:2) (2P) SM(42:1) (2Q) SM(40:3).

FIG. 3A depicts immunoblots for Cav-1 in PC-3M cells following 72 hour treatment with SFM or lipid-containing SFM. SSALPs of defined lipid composition were generated and spiked into media. sLDL: synthetic ‘LDL-like’ particles; sHDL: synthetic ‘HDL-like’ particles; PC—phosphatidylcholine; TO—trioleate; CE: cholesteryl oleate; FC: Free cholesterol. (i) SFM (vehicle) (ii) sHDL (PC/TO^{low}) (iii) sHDL (PC/CE/TO^{high}) (iv) sHDL (PC/FC/CE/TO^{high}) (v) sHDL (PC/TO^{high}) (vi) sHDL (PC/SM/CE/TO^{high}) (vii) sHDL (PC/SM/FC/CE/TO^{high}) (viii) sHDL (PC/CE/TO^{high}) (ix) sHDL (PC/SM/TO^{low}) (x) sHDL (PC/SM/FC/TO^{high}) (xi) SFM (Control) (xii) sHDL (PC/SM/FC/CE/TO^{low}) (xiii) sHDL (PC/SM/CE/TO^{low}) (xiv) SFM (Vehicle). FIG. 3B depicts relative lipid composition of SSALPs. (i) LDL (PC/TO^{high}) (ii) LDL (PC/CE/TO^{high}) (iii) LDL (PC/SM/TO^{high}) (iv) LDL (PC/SM/CE/TO^{high}) (V) LDL (PC/SM/FC/TO^{high}) (vi) LDL (PC/SM/FC/CE/TO^{high}) (vii) HDL (PC/SM/TO^{low}) (viii) HDL (PC/SM/CE/TO^{low}) (ix) HDL (PC/SM/FC/TO^{low}) (x) HDL (PC/SM/FC/TO^{low}). FIG. 3C depicts representative immunoblots for Cav-1 in LNCaP (left) and PC-3M (right) prostate cancer cells following Cav-1 overexpression or knockdown, respectively. FIG. 3D depicts baseline assessment of Dil-SSALP uptake by LNCaP, PC-3M and RM-9 prostate cancer cells pretreated with Dil-SSALPs.

FIG. 4A depicts fold change (vertical axis, relative to median of cell line-specific control) in lipid domains following overexpression or transient knockdown of CAV-1 in LNCaP and PC-3M, respectively. (i) acylcarnitines (ii) cardiolipins (iii) ceramides (iv) cholesterol esters (v) diacylglycerols (vi) glycosphingolipids (vii) lysophospholipids (viii) phospholipids (ix) sphingomyelins (x) triacylglycerols. For lipid domains, the aggregate intensity of individual annotated lipid species corresponding to the respective lipid domain was used. Statistical significance was determined by 2-sided student t-test. FIG. 4B depicts relative abundance (area units±StDev) of lactosylceramides following overexpression of CAV-1 in LNCaP or transient knockdown of CAV-1 in PC-3M. (i) lactosylceramide(30:1) (ii) lactosylceramide(18:1/20:4) (iii) lactosylceramide(18:1/16:0). Statistical significance was determined by 2-sided student t-test. Lipid abundances were normalized based on total cell number.

FIGS. 5A and 5B shows biochemical networks illustrating gene expression of enzymes central to ceramide metabolism in prostate cancer cell lines (5A) and prostate adenocarcinomas (5B) stratified by high or low CAV-1 expression. For CCLE data (5A), prostate cancer cell lines were stratified by mean CAV1 mRNA expression into either high (log 2 mRNA range: 11.01-13.61) or low (log 2 mRNA range 4.16-6.88) CAV1 expression. For TCGA data (5B), prostate adenocarcinomas were stratified into the highest or lowest CAV-1 expression quartiles. Node size reflects magnitude of change. Edges and arrows illustrate direction of biochemical reactions. Thickened black node borders indicates statistically significant differences.

FIG. 6 provides an overview of ceramide biosynthetic pathways.

FIG. 7A depicts a schematic illustrating potential biochemical fates of sphingomyelin(18:1/18:1)-d₉. FIG. 7B depicts relative abundance (Area units) for (i) sphingomyelin(18:1/18:1), (ii) ceramide(18:1/18:1), (iii) glucosylceramide(18:1/18:1) as well as their deuterated (d₉) isotopologues (iv, v, and vi, respectively) in LNCaP, PC-3M and RM-9 following 48 hour treatment with SSALPs-enriched in sphingomyelin(18:1/18:1)-d₉. Values presented above bar plots indicate the ratio between the ceramide(18:1/18:1)-d₉ and sphingomyelin(18:1/18:1)-d₉.

FIGS. 8A, 8B, and 8C show the relationship between CAV-1 and mitochondrial morphology. (8A) Representative images of mitochondria and lysosomes in PC-3M (upper) and LNCaP (lower) cells transfected with CellLight Lysosome-GFP (lysosomal associated membrane protein 1) and CellLight Mitochondria-RFP (leader sequence of E1 alpha pyruvate dehydrogenase). (8B) Uptake of C11 TopFluor-SM in PC-3M cells following knockdown of CAV1. (8C) Violin plots illustrating intensities of TopFluor-SM in PC-3M cells following knockdown of CAV1. Vertical axis=RFU±SEM. Statistical significance was determined using One-way ANOVA; pairwise comparisons were performed using Tukey HSD multiple comparison test and adjusted p-value reported. (i) siCtrl (ii) Mock (iii) siCAV-1 (iv) siCAV-2.

FIGS. 9A, 9B, 9C, and 9D show representative images from staining for (i) lysosomes (CellLight Lysosome-GFP (lysosomal associated membrane protein 1)), (ii) mitochondria (CellLight Mitochondria-RFP (leader sequence of E1 alpha pyruvate dehydrogenase), and (iii) merged image, in PC-3M cells following knockdown of CAV1.

FIGS. 10A, 10B, and 10C show representative images from (10A) staining for mitochondria mass (MitoTracker Green) in PC-3M cells following knockdown of CAV1. Scale bar indicates 20 μm. Intensity scale bars are provided next to each figure. Also shown are violin plots illustrating intensities of MitoTracker Green (10B) and MitoTracker CMXRos (10C) in PC-3M cells following knockdown of CAV1. Vertical axis=RFU±SEM. Statistical significance was determined using One-way ANOVA; pairwise comparisons were performed using Tukey HSD multiple comparison test and adjusted p-value reported.

FIGS. 11A and 11B show representative images from (11A) co-staining for (ii) CAV1 (FITC) and (iii) mitochondrial potential/reactive oxygen species (MitoTracker Red CMXRos), and and (i) mergedi mamges, in PC-3M cells following knockdown of CAV1. (11B) Intracellular levels of reactive oxygen species assessed via CellROX Deep red in PC-3M cells following knockdown of CAV1. (i) siCtrl (ii) ciCAV1-1.

FIGS. 12A, 12B, and 12C show secretion of Cav-1 containing extracellular vesicles, enriched in sphingomyelins and lactosylceramides, upon elevated CAV1 expression. (12A) Schematic of multi-fractionation approach. (12B) Cav-1 levels (ng/mL) in extracellular vesicles derived from conditioned media of LNCaP and PC-3M following overexpression of Cav-1 or transient knockdown of CAV1 in the presence or absence of BSA or human-derived lipoproteins, respectively. (i) media+serum-lipoproteins+LDL (ii) media+serum-lipoproteins+BSA (iii) media+serum-lipoproteins. Vertical axis=ng CAV1/mL. (12C) Particle counts per mL (vertical axis) from conditioned media of (i) LNCaP and (ii) PC-3M following overexpression of Cav-1 or knockdown of CAV1, respectively. Horizontal axis=size/nm. Statistical significance was determined by 2-sided Student T-test comparing the area under the curve following Cav-1 overexpression or knockdown relative to the respective scramble controls.

FIGS. 13A, 13B, 13C, and 13D show levels of sphingomyelins (13A) and lactosylceramides (13B) in conditioned media of LNCaP and PC-3M following overexpression of Cav-1 or transient knockdown of CAV-1 in the presence or absence of BSA or human-derived lipoproteins, respectively. (i) base media (ii) CAV(NC1) (iii) CAV(si8) (iv) CAV− (v) CAV+ (vi) base media+BSA (vii) CAV(NC1)/BSA (viii) CAV(si8)/BSA (ix) CAV−/BSA (x) CAV+/BSA (xi) base media+LDL (xii) CAV(NC1)/LDL (xiii) CAV(si8)/LDL (xiv) CAV−/LDL (xv) CAV+/LDL. (13C) Lipid composition of Evs isolated from conditioned media of LNCaP (left) and PC-3M (right) prostate cancer cells. (i) acylcarnitine (ii) oxylipin (iii) lysophospholipid (iv) phospholipid (v) sphingomyelins (vi) ceramide (vii) glycosphingolipid (viii) cardiolipin (ix) monoacylglycerol (x) diacylglycerol (xi) triacylglycerol (xii) cholesterol ester. (13D) Proteins in PC-3M-derived EVs that are annotated as localized to the mitochondria based on the COMPARTMENTS localization evidence database scores. (i) peroxisome (ii) Golgi apparatus (iii) endosome (iv) lysosome (v) endoplasmic reticulum (vi) mitochondrion (vii) cytoskeleton (viii) extracellular region (ix) plasma membrane (x) nucleus (xi) cytosol.

FIGS. 14A and 14B show heatmaps illustrating subcellular localization of protein features identified in EVs isolated from conditioned media of (14A) PC-3M or (14B) LNCaP prostate cancer cells. Subcellular localization is based on the COMPARTMENTS localization evidence database scores. (i) peroxisome (ii) Golgi apparatus (iii) endosome (iv) lysosome (v) endoplasmic reticulum (vi) mitochondrion (vii) cytoskeleton (viii) extracellular region (ix) plasma membrane (x) nucleus (xi) cytosol.

FIGS. 15A, 15B, and 15C show (15A) viability curves for (left) RM-9 and (right) PC-3M cells following 48 hour treatment with control, (i) PPMP, (ii) PDMP, or (iii) Eliglustat. Horizontal axis=log(M); vertical axis=% viability relative to control. Also shown are relative abundances of (15B) ceramides and (15C) glycosphingolipids following 6 hr treatment with (left) RM-9 and (right) PC-3M with either vehicle (ethanol) or inhibitor. (i) vehicle (ii) 128 μM Eliglustat (iii) 64 μM PDMP (iv) 64 μM PPMP. Statistical significance was determined by 2-sided student t-test comparing the aggregate intensities of individual lipid species corresponding to the respective lipid domain.

FIGS. 16A, 16B, and 16C show (16A) the induction of autophagy/mitophagy by exposure of PC-3M prostate cancer cells to Eliglustat. (i) cytotoxicity (ii) apoptosis at 4 hrs (iii) apoptosis at 24 hrs. N=3 biologically independent replicates per experimental condition. Values represent log(relative fluorescence units)±StDev. Also shown are volcano plots illustrating differences in annotated lipid species stratified by lipid domains in (16B) PC-3M and (16C) RM-9 prostate cancer cells following 6 hr challenge with 128 μM Eliglustat. (i) acylcarnitines (ii) cardiolipins (iii) diacylglycerols (iv) ether lysophospholipids (v) ether phospholipids (vi) lysophospholipids (vii) phospholipids (viii) cholesterol esters (ix) triacylglycerols (x) sphingomyelins. N=3 biologically independent replicates per experimental condition. Statistical significance was determined by 2-sided student t-test.

FIGS. 17A, 17B, 17C, and 17D show representative confocal microscopy images for PC-3M cells following 24 hr pre-transfection with (i) CellLight Mitochondria-RFP (leader sequence of E1 alpha pyruvate dehydrogenase) or (ii) CellLight Lysosome-GFP (lysosomal associated 1052 membrane protein 1) followed by acute (6 hr) treatment with either (17A) vehicle or (17C) Eliglustat (128 μM). (17B) and (17D) correspond to enlargements of features from (17A) and (17C), respectively. (iii) merged images.

FIGS. 18A and 18B show (18A) the viability (MTS assay) of PC-3M cells treated with 128 μM eliglustat after (left) knockdown of CAV1: or (right) following pre-treatment with Cav-1 monoclonal blocking antibody: (i) siCtrl (ii) siCAV1-1 (iii) siCAV1-2 (iv) IgG=vehicle (v) IgG+Eliglustat (vi) abCAV1+Vehicle (vii) abCAV1+Eliglustat. (18B) Viability (MTS assay) of LNCaP cells treated with 64 μM eliglustat following Cav-1 overexpression.

FIGS. 19A, 19B, 19C, and 19D show the efficacy of eliglustat in an in vivo mouse model. (19A) Relative fluorescence units±SEM of RM-9 tumors following daily intraperitoneal injection with either (i) saline (n=23) or (ii) eliglustat (60 mg/kg) (n=9). (19B) Tumor Volume±SEM following treatment with either saline (n=15) or eliglustat (60 mg/kg) (n=8). Statistical significance was determined by 2-sided Wilcoxon-rank sum test. (19C) Representative IVIS images following treatment. (19D) Relative abundance (area units±StDev) of glycosphinoglipids in RM-9 tumors following treatment. Statistical significance was determined by 2-sided Wilcoxon-rank sum test comparing the aggregate intensities of individual lipid species corresponding to the respective lipid domain.

FIGS. 20A, 20B, 20C, 20D, 20E, and 20F show quantitative analyses derived from immunohistochemistry staining for (20A) Cav-1, (20B) BrdU-TUNEL, (20C) PCNA, (20D) HMGB1 and (20E) LC3B in RM-9 tumors following treatment with either saline (n=6-8 mice) or eliglustat (n=7 mice). Vertical axes are (20A) Cav-1 staining score, (20B) apoptotic bodies/fd, (20C) % of PCNA labeling, (20D) % of cytoplasmic HMGB1, and (20E) LC-3B positive cells/fd. Statistical significance was determined by 2-sided Wilcoxon rank sum test. Also shown is (20F) volcano plot illustrating fold change in individual annotated lipid species stratified by lipid domain in plasma of RM-9 bearing C57BL/6N mice or control mice. (i) acylcarnitines (ii) ceramides (iii) cholesterol esters (iv) diacylglycerols (v) free fatty acids (vi) glycosphingolipids (vii) lysophospholipids (viii) oxylipins (ix) phospholipids (x) sphingomyelins (x) triacylglycerols.

FIG. 21 depicts odds ratios (95% CI) for per unit increase for TrihexosylCer(34:1), LactosylCer(36:0), LactosylCer(32:0), SM(44:2), SM(40:2), the plasma sphingolipid signature (SphingoSignature), and a simplified sphingolipid signature (Simplified Signature) for assessing risk of disease progression among men on active surveillance for prostate cancer in an independent validation cohort consisting of 248 participants (35 progressors, 213 non progressors). * indicates statistical significance, 1-sided p<0.05.

### DETAILED DESCRIPTION

In one aspect, the disclosure provides a method of determining the risk for progression of prostate cancer in a subject, comprising performing the following steps on a biological sample obtained from the subject: measuring the level of CAV-1 in the biological sample, measuring the level of SM(40:2) in the biological sample, measuring the level of SM(44:2) in the biological sample, measuring the level of LacCer 32:0 in the biological sample, measuring the level of LacCer 36:0 in the biological sample, measuring the level of TriHexCer 34:1 in the biological sample, and measuring the level of HexCer 40:0 in the biological sample; wherein the amount of CAV-1, SM(40:2), SM(44:2), LacCer 32:0, LacCer 36:0, TriHexCer 34:1 and HexCer 40:0 determines the risk for progression of prostate cancer in the subject.

In another aspect, the disclosure provides a method of determining the risk for progression of prostate cancer in a subject, comprising performing the following steps on a biological sample obtained from the subject: contacting the sample with a first reporter molecule that binds CAV-1, a second reporter molecule that binds SM(40:2), a third reporter molecule that binds SM(44:2), a fourth reporter molecule that binds LacCer 32:0, a fifth reporter molecule that binds LacCer 36:0, a sixth reporter molecule that binds TriHexCer 34:1, and a seventh reporter molecule that binds HexCer 40:0; wherein the amount of the first reporter molecule, the second reporter molecule, the third reporter molecule, the fourth reporter molecule, the fifth reporter molecule, the sixth reporter molecule, and the seventh reporter molecule determines the risk for progression of prostate cancer in the subject.

In another aspect, the disclosure provides a method of determining the risk for progression of prostate cancer in a subject, comprising performing the following steps on a biological sample obtained from the subject: performing spectrometric analysis for CAV-1, performing spectrometric analysis for SM(40:2), performing spectrometric analysis for SM(44:2), performing spectrometric analysis for LacCer 32:0, performing spectrometric analysis for LacCer 36:0, performing spectrometric analysis for TriHexCer 34:1, performing spectrometric analysis for HexCer 40:0, wherein the spectrometric analyses determine the risk for progression of prostate cancer in the subject. In some aspects, the spectrometric analyses are quantitative analyses. In some aspects, the spectrometric analyses are mass spectrometric analyses. In some aspects, the spectrometric analyses are performed concurrently. In some aspects, the spectrometric analyses are performed sequentially. In some aspects, the method further comprises a chromatographic step. In some aspects, the method further comprises a liquid chromatographic step. In some aspects, the method further comprises a high performance liquid chromatographic (“HPLC”) step. In some aspects, the method further comprises a gas chromatographic (“GC”) step. In some aspects, the chromatographic step is coupled directly to the spectrometric step. In some aspects, the chromatographic step separates at least one analyte from at least one other analyte.

In another aspect, the disclosure provides a method of determining the risk for progression of prostate cancer in a subject, comprising performing the following steps on a biological sample obtained from the subject: providing a surface that binds CAV-1, SM(40:2), SM(44:2), LacCer 32:0, LacCer 36:0, TriHexCer 34:1 and HexCer 40:0; incubating the surface with the biological sample; contacting the surface with a first reporter molecule that binds CAV-1, contacting the surface with a second reporter molecule that binds SM(40:2), contacting the surface with a third reporter molecule that binds SM(44:2), contacting the surface with a fourth reporter molecule that binds LacCer 32:0, contacting the surface with a fifth reporter molecule that binds LacCer 36:0, contacting the surface with a sixth reporter molecule that binds TriHexCer 34:1, and contacting the surface with a seventh reporter molecule that binds HexCer 40:0; measuring the amount of the first reporter molecule that is associated with the surface; measuring the amount of the second reporter molecule that is associated with the surface; measuring the amount of the third reporter molecule that is associated with the surface; measuring the amount of the fourth reporter molecule that is associated with the surface; measuring the amount of the fifth reporter molecule that is associated with the surface; measuring the amount of the sixth reporter molecule that is associated with the surface; measuring the amount of the seventh reporter molecule that is associated with the surface; wherein the amount of the first reporter molecule, the second reporter molecule, the third reporter molecule, the fourth reporter molecule, the fifth reporter molecule, the sixth reporter molecule, and the seventh reporter molecule, determines the risk for progression of prostate cancer in the subject.

In another aspect, the disclosure provides a method of determining the risk for progression of prostate cancer in a subject, comprising performing the following steps on a biological sample obtained from the subject: providing a first surface with means for binding CAV-1, providing a second surface with means for binding SM(40:2), providing a third surface with means for binding SM(44:2), providing a fourth surface with means for binding LacCer 32:0, providing a fifth surface with means for binding LacCer 36:0, providing a sixth surface with means for binding TriHexCer 34:1, and providing a seventh surface with means for binding HexCer 40:0; incubating the first surface with the biological sample; incubating the second surface with the biological sample; incubating the third surface with the biological sample; incubating the fourth surface with the biological sample; incubating the fifth surface with the biological sample; incubating the sixth surface with the biological sample; incubating the seventh surface with the biological sample; contacting the first surface with a first reporter molecule that binds CAV-1, contacting the second surface with a first reporter molecule that binds SM(40:2), contacting the third surface with a first reporter molecule that binds SM(44:2), contacting the fourth surface with a first reporter molecule that binds LacCer 32:0, contacting the fifth surface with a first reporter molecule that binds LacCer 36:0, contacting the sixth surface with a first reporter molecule that binds TriHexCer 34:1, and contacting the seventh surface with a first reporter molecule that binds HexCer 40:0; measuring the amount of the first reporter molecule associated with the first surface; measuring the amount of the second reporter molecule associated with the second surface; measuring the amount of the third reporter molecule associated with the third surface; measuring the amount of the fourth reporter molecule associated with the fourth surface; measuring the amount of the fifth reporter molecule associated with the fifth surface; measuring the amount of the sixth reporter molecule associated with the sixth surface; measuring the amount of the seventh reporter molecule associated with the seventh surface; wherein the amount of the first reporter molecule, the second reporter molecule, the third reporter molecule, the fourth reporter molecule, the fifth reporter molecule, the sixth reporter molecule, and the seventh reporter molecule determines the risk for progression of prostate cancer in the subject.

In another aspect, the disclosure provides a method of determining the risk for progression of prostate cancer in a subject, comprising performing the following steps on a biological sample obtained from the subject: providing a surface with means for binding CAV-1, SM(40:2), SM(44:2), LacCer 32:0, LacCer 36:0, TriHexCer 34:1 and HexCer 40:0; incubating the surface with the biological sample; contacting the surface with a first relay molecule that binds CAV-1, contacting the surface with a second relay molecule that binds SM(40:2), contacting the surface with a third relay molecule that binds SM(44:2); contacting the surface with a fourth relay molecule that binds LacCer 32:0; contacting the surface with a fifth relay molecule that binds LacCer 36:0; contacting the surface with a sixth relay molecule that binds TriHexCer 34:1; and contacting the surface with a seventh relay molecule that binds HexCer 40:0; contacting the surface with a first reporter molecule that binds to the first relay molecule; contacting the surface with a second reporter molecule that binds to the second relay molecule; contacting the surface with a third reporter molecule that binds to the third relay molecule; contacting the surface with a fourth reporter molecule that binds to the fourth relay molecule; contacting the surface with a fifth reporter molecule that binds to the fifth relay molecule; contacting the surface with a sixth reporter molecule that binds to the sixth relay molecule; contacting the surface with a seventh reporter molecule that binds to the seventh relay molecule; measuring the amount of the first reporter molecule associated with the first relay molecule and CAV-1; measuring the amount of the second reporter molecule associated with the second relay molecule and SM(40:2); measuring the amount of the third reporter molecule associated with the third relay molecule and SM(44:2); measuring the amount of the fourth reporter molecule associated with the fourth relay molecule and LacCer 32:0; measuring the amount of the fifth reporter molecule associated with the fifth relay molecule and LacCer 36:0; measuring the amount of the sixth reporter molecule associated with the sixth relay molecule and TriHexCer 34:1; and measuring the amount of the seventh reporter molecule associated with the seventh relay molecule and HexCer 40:0; wherein the amount of the first reporter molecule, the second reporter molecule, the third reporter molecule, the fourth reporter molecule, the fifth reporter molecule, the sixth reporter molecule, and the seventh reporter molecule determines the risk for progression of prostate cancer in the subject.

In one embodiment, the amounts of CAV-1, SM(40:2), SM(44:2), LacCer 32:0, LacCer 36:0, TriHexCer 34:1 and HexCer 40:0 or the reporter molecules bound thereto are elevated in the subject relative to a healthy subject. In one embodiment, the amounts of CAV-1, SM(40:2), SM(44:2), LacCer 32:0, LacCer 36:0, TriHexCer 34:1 and HexCer 40:0 or the reporter molecules bound thereto are elevated in the subject relative to a subject without prostate cancer. In one embodiment, the amounts of CAV-1, SM(40:2), SM(44:2), LacCer 32:0, LacCer 36:0, TriHexCer 34:1 and HexCer 40:0 or the reporter molecules bound thereto are elevated in the subject relative to a subject with indolent prostate cancer.

In another embodiment, at least one of the reporter molecules provides a detectable signal. In another embodiment, the detectable signal is detectable by a method selected from UV-visible spectroscopy, mass spectrometry, nuclear magnetic resonance (NMR) spectroscopy, proton NMR spectroscopy, nuclear magnetic resonance (NMR) spectrometry, gas chromatography-mass spectrometry (GC-MS), liquid chromatography-mass spectrometry (LC-MS), correlation spectroscopy (COSy), nuclear Overhauser effect spectroscopy (NOESY), rotating frame nuclear Overhauser effect spectroscopy (ROESY), LC-TOF-MS, LC-MS/MS, and capillary electrophoresis-mass spectrometry. In another embodiment, the spectrometric method is mass spectrometry. In another embodiment, the panel comprises biomarkers that have been identified by a method selected from UV-visible spectroscopy, mass spectrometry, nuclear magnetic resonance (NMR) spectroscopy, proton NMR spectroscopy, nuclear magnetic resonance (NMR) spectrometry, gas chromatography-mass spectrometry (GC-MS), liquid chromatography-mass spectrometry (LC-MS), correlation spectroscopy (COSy), nuclear Overhauser effect spectroscopy (NOESY), rotating frame nuclear Overhauser effect spectroscopy (ROESY), LC-TOF-MS, LC-MS/MS, and capillary electrophoresis-mass spectrometry. In another embodiment, the panel comprises biomarkers that have been identified by UV-visible spectroscopy or proton NMR spectroscopy.

In another embodiment, the first reporter binds selectively to CAV-1. In another embodiment, the second reporter binds selectively to SM(40:2). In another embodiment, the third reporter binds selectively to SM(44:2). In another embodiment, the fourth reporter binds selectively to LacCer 32:0. In another embodiment, the fifth reporter binds selectively to LacCer 36:0. In another embodiment, the sixth reporter binds selectively to TriHexCer 34:1. In another embodiment, the seventh reporter binds selectively to HexCer 40:0.

In another embodiment, determination of CAV-1, SM(40:2), SM(44:2), LacCer 32:0, LacCer 36:0, TriHexCer 34:1 and HexCer 40:0 levels is made at substantially the same time. In another embodiment, determination of CAV-1, SM(40:2), SM(44:2), LacCer 32:0, LacCer 36:0, TriHexCer 34:1 and HexCer 40:0 levels is made in a stepwise manner. In another embodiment, such methods comprise inclusion of subject history information into the determination of risk for progression of prostate cancer. In another embodiment, such methods comprise administering at least one alternate diagnostic test for a subject assigned as being at risk for risk for progression of prostate cancer.

In another aspect, the disclosure provides a method of treating a subject suspected of being at risk for progression of prostate cancer, comprising analyzing the subject for risk for progression of prostate cancer with a method as described herein, and administering a therapeutically effective amount of a treatment for the cancer. In one embodiment, the treatment is surgery, chemotherapy, immunotherapy, radiation therapy, targeted therapy, or a combination thereof.

In another aspect, the disclosure provides a method of determining the risk for progression of prostate cancer in a subject, comprising performing the following steps on a biological sample obtained from the subject:
measuring the level of CAV-1 in the biological sample; measuring the level of SM(40:2) in the biological sample; measuring the level of SM(44:2) in the biological sample; measuring the level of LacCer 32:0 in the biological sample; measuring the level of LacCer 36:0 in the biological sample; measuring the level of TriHexCer 34:1 in the biological sample; measuring the level of pro-SFTPB in the biological sample; determining the level of CAV-1 relative to a first standard value, wherein the ratio is predictive of the risk for progression of prostate cancer; determining the level of SM(40:2) relative to a second standard value, wherein the ratio is predictive of the risk for progression of prostate cancer; determining the level of SM(44:2) relative to a third standard value, wherein the ratio is predictive of the risk for progression of prostate cancer; determining the level of LacCer 32:0 relative to a fourth standard value, wherein the ratio is predictive of the risk for progression of prostate cancer; determining the level of LacCer 36:0 relative to a fifth standard value, wherein the ratio is predictive of the risk for progression of prostate cancer; determining the level of TriHexCer 34:1 relative to a sixth standard value, wherein the ratio is predictive of the risk for progression of prostate cancer; determining the level of HexCer 40:0 relative to a seventh standard value, wherein the ratio is predictive of the risk for progression of prostate cancer; and assigning a risk for progression of prostate cancer or not at risk for progression of prostate cancer, as determined by statistical analysis of the ratios of CAV-1, SM(40:2), SM(44:2), LacCer 32:0, LacCer 36:0, TriHexCer 34:1 and HexCer 40:0 levels.

In another aspect, the disclosure provides a method of predicting the risk for progression of prostate cancer in a subject, comprising performing the following steps on a biological sample from the subject obtained from the subject: measuring the levels of the CAV-1, SM(40:2), SM(44:2), LacCer 32:0, LacCer 36:0, TriHexCer 34:1 and HexCer 40:0 biomarkers in the biological sample; and calculating a predictive factor as determined by statistical analysis of the CAV-1, SM(40:2), SM(44:2), LacCer 32:0, LacCer 36:0, TriHexCer 34:1 and HexCer 40:0 levels.

In another aspect, the disclosure provides a method of determining the risk for progression of prostate cancer in a subject, comprising performing the following steps on a biological sample obtained from the subject: measuring the levels of CAV-1, SM(40:2), SM(44:2), LacCer 32:0, LacCer 36:0, TriHexCer 34:1 and HexCer 40:0 biomarkers in the biological sample; assigning the condition of the subject as either at risk for progression of prostate cancer or not at risk for progression of prostate cancer, as determined by statistical analysis of the levels of CAV-1, SM(40:2), SM(44:2), LacCer 32:0, LacCer 36:0, TriHexCer 34:1 and HexCer 40:0 in the biological sample, and determining, from analysis of the levels of CAV-1, SM(40:2), SM(44:2), LacCer 32:0, LacCer 36:0, TriHexCer 34:1 and HexCer 40:0, a risk for progression of prostate cancer.

In another embodiment, the first reporter binds selectively to CAV-1. In another embodiment, the second reporter binds selectively to SM(40:2). In another embodiment, the third reporter binds selectively to SM(44:2). In another embodiment, the fourth reporter binds selectively to LacCer 32:0. In another embodiment, the fifth reporter binds selectively to LacCer 36:0. In another embodiment, the sixth reporter binds selectively to TriHexCer 34:1. In another embodiment, the seventh reporter binds selectively to HexCer 40:0. In another embodiment, determination of CAV-1, SM(40:2), SM(44:2), LacCer 32:0, LacCer 36:0, TriHexCer 34:1 and HexCer 40:0 levels is made at substantially the same time. In another embodiment, determination of CAV-1, SM(40:2), SM(44:2), LacCer 32:0, LacCer 36:0, TriHexCer 34:1 and HexCer 40:0 levels is made in a stepwise manner. In another embodiment, such methods further comprise inclusion of subject history information into the assignment of being at risk for progression of prostate cancer or not being at risk for progression of prostate cancer. In another embodiment, such methods comprise administering at least one alternate diagnostic test for a subject assigned as being at risk for progression of prostate cancer.

In another aspect, the disclosure provides a method of treating a subject suspected of being at risk for progression of prostate cancer, comprising analyzing the subject for risk for progression of prostate cancer with a method as described herein; and administering a therapeutically effective amount of a treatment for the cancer. In another embodiment, the treatment is surgery, chemotherapy, immunotherapy, radiation therapy, targeted therapy, or a combination thereof. In another embodiment, the classification of the subject as being at risk for progression of prostate cancer has a sensitivity of 0.76 and 0.42 at 78% and 94% specificity, respectively.

In another embodiment, the levels of CAV-1, SM(40:2), SM(44:2), LacCer 32:0, LacCer 36:0, TriHexCer 34:1 and HexCer 40:0 are elevated in comparison to the levels of CAV-1, SM(40:2), SM(44:2), LacCer 32:0, LacCer 36:0, TriHexCer 34:1 and HexCer 40:0 in a reference subject or group that has adenocarcinoma.

In another embodiment, the levels of CAV-1, SM(40:2), SM(44:2), LacCer 32:0, LacCer 36:0, TriHexCer 34:1 and HexCer 40:0 are elevated in comparison to the levels of CAV-1, SM(40:2), SM(44:2), LacCer 32:0, LacCer 36:0, TriHexCer 34:1 and HexCer 40:0 in a reference subject or group that has squamous cell cancer.

In another aspect, the disclosure provides a kit for the method comprising a reagent solution that comprises a first solute for detection of CAV-1; a second solute for detection of SM(40:2); a third solute for detection of SM(44:2); a fourth solute for detection of LacCer 32:0; a fifth solute for detection of LacCer 36:0; a sixth solute for detection of TriHexCer 34:1; and a seventh solute for detection of HexCer 40:0.

In another embodiment, such methods further comprise a device for contacting the reagent solutions with a biological sample. In another embodiment, such methods comprise at least one surface with means for binding at least one biomarker. In another embodiment, the at least one biomarker is selected from the group consisting of CAV-1, SM(40:2), SM(44:2), LacCer 32:0, LacCer 36:0, TriHexCer 34:1 and HexCer 40:0.

In another aspect, the disclosure provides a method of determining the risk for progression of prostate cancer in a subject, comprising a biomarker panel and a protein marker panel: wherein the biomarker panel comprises CAV-1, SM(40:2), SM(44:2), LacCer 32:0, LacCer 36:0, TriHexCer 34:1 and HexCer 40:0; wherein the method comprises: performing the following steps on obtaining a biological sample from the subject; measuring the levels of the biomarkers and the protein biomarkers in the biological sample; wherein the amount of the biomarkers and the protein biomarkers determines the risk for progression of prostate cancer in the subject.

In another aspect, the disclosure provides a method of determining the risk for progression of prostate cancer in a subject, comprising performing the following steps on obtaining a biological sample from the subject; measuring the levels of CAV-1, SM(40:2), SM(44:2), LacCer 32:0, LacCer 36:0, TriHexCer 34:1 and HexCer 40:0 in the biological sample; and determining the risk for progression of prostate cancer in the subject, as determined by statistical analysis of the levels of CAV-1, SM(40:2), SM(44:2), LacCer 32:0, LacCer 36:0, TriHexCer 34:1 and HexCer 40:0 in the biological sample. In one embodiment, the levels of CAV-1, SM(40:2), SM(44:2), LacCer 32:0, LacCer 36:0, TriHexCer 34:1 and HexCer 40:0 or the reporter molecules bound thereto are elevated in the subject relative to a healthy subject. In another embodiment, the levels of CAV-1, SM(40:2), SM(44:2), LacCer 32:0, LacCer 36:0, TriHexCer 34:1 and HexCer 40:0 are elevated in comparison to the levels of CAV-1, SM(40:2), SM(44:2), LacCer 32:0, LacCer 36:0, TriHexCer 34:1 and HexCer 40:0 in a reference subject or group that does not have prostate cancer. In another embodiment, the reference subject or group is healthy. In another embodiment, such methods comprise at least one receptor molecule that selectively binds to a biomarker selected from the group consisting of CAV-1, SM(40:2), SM(44:2), LacCer 32:0, LacCer 36:0, TriHexCer 34:1 and HexCer 40:0. In another embodiment, the sample comprises a biological sample selected from blood, plasma, and serum. In another embodiment, the biological sample is serum. In another embodiment, the amount of CAV-1, SM(40:2), SM(44:2), LacCer 32:0, LacCer 36:0, TriHexCer 34:1 and HexCer 40:0 is quantified. In another embodiment, detection of the amount of CAV-1, SM(40:2), SM(44:2), LacCer 32:0, LacCer 36:0, TriHexCer 34:1 and HexCer 40:0 comprises the use of a solid particle. In another embodiment, the solid particle is a bead. In another embodiment, at least one of the reporter molecules is linked to an enzyme. In another embodiment, at least one of the reporter molecules provides a detectable signal. In another embodiment, the detectable signal is detectable by a method selected from UV-visible spectroscopy, mass spectrometry, nuclear magnetic resonance (NMR) spectroscopy, proton NMR spectroscopy, nuclear magnetic resonance (NMR) spectrometry, gas chromatography-mass spectrometry (GC-MS), liquid chromatography-mass spectrometry (LC-MS), correlation spectroscopy (COSy), nuclear Overhauser effect spectroscopy (NOESY), rotating frame nuclear Overhauser effect spectroscopy (ROESY), LC-TOF-MS, LC-MS/MS, and capillary electrophoresis-mass spectrometry. In another embodiment, the concentrations of CAV-1, SM(40:2), SM(44:2), LacCer 32:0, LacCer 36:0, TriHexCer 34:1 and HexCer 40:0 are measured. In another embodiment, the subject is determined to be at risk for progression of prostate cancer based on the measured concentrations of the biomarkers. In another embodiment, the measured concentrations are used to calculate a biomarker score based on sensitivity and specificity values at a given cutoff. In another embodiment, such methods further comprise the steps of: comparing the measured concentrations of each biomarker in the biological sample to the prediction of a statistical model. In another embodiment, the panel is selected from the group consisting of: a. the panel consisting of CAV-1, SM(40:2), SM(44:2), LacCer 32:0, LacCer 36:0, TriHexCer 34:1 and HexCer 40:0; or b. the panel consisting of CAV-1, SM(40:2), SM(44:2), LacCer 32:0, LacCer 36:0, TriHexCer 34:1 and HexCer 40:0. In another embodiment, the panel comprises biomarkers that have been identified by a method selected from UV-visible spectroscopy, mass spectrometry, nuclear magnetic resonance (NMR) spectroscopy, proton NMR spectroscopy, nuclear magnetic resonance (NMR) spectrometry, gas chromatography-mass spectrometry (GC-MS), liquid chromatography-mass spectrometry (LC-MS), correlation spectroscopy (COSy), nuclear Overhauser effect spectroscopy (NOESY), rotating frame nuclear Overhauser effect spectroscopy (ROESY), LC-TOF-MS, LC-MS/MS, and capillary electrophoresis-mass spectrometry. In another embodiment, the panel comprises biomarkers that have been identified by UV-visible spectroscopy or proton NMR spectroscopy.

In another embodiment, the first reporter binds selectively to CAV-1. In another embodiment, the second reporter binds selectively to SM(40:2). In another embodiment, the third reporter binds selectively to SM(44:2). In another embodiment, the fourth reporter binds selectively to LacCer 32:0. In another embodiment, the fifth reporter binds selectively to LacCer 36:0. In another embodiment, the sixth reporter binds selectively to TriHexCer 34:1. In another embodiment, the seventh reporter binds selectively to HexCer 40:0.

In another embodiment, determination of CAV-1, SM(40:2), SM(44:2), LacCer 32:0, LacCer 36:0, TriHexCer 34:1 and HexCer 40:0 levels is made at substantially the same time. In another embodiment, determination of CAV-1, SM(40:2), SM(44:2), LacCer 32:0, LacCer 36:0, TriHexCer 34:1 and HexCer 40:0 levels is made in a stepwise manner. In another embodiment, such methods further comprise inclusion of subject history information into the assignment of being at risk for progression of prostate cancer or not being at risk for progression of prostate cancer. In another embodiment, such methods comprise administering at least one alternate diagnostic test for a subject assigned as being at risk for progression of prostate cancer.

In another embodiment, the disclosure provides a kit for the method as described herein, comprising: a reagent solution that comprises a first solute for detection of CAV-1; a second solute for detection of SM(40:2); a third solute for detection of SM(44:2); a fourth solute for detection of LacCer 32:0; a fifth solute for detection of LacCer 36:0; a sixth solute for detection of TriHexCer 34:1; and a seventh solute for detection of HexCer 40:0

In another aspect, the disclosure provides a kit for a method as described herein, comprising a first reagent solution that comprises a first solute for detection of CAV-1, a second reagent solution that comprises a second solute for detection of SM(40:2), a third reagent solution that comprises a third solute for detection of SM(44:2), a fourth reagent solution that comprises a fourth solute for detection of LacCer 32:0, a fifth reagent solution that comprises a fifth solute for detection of LacCer 36:0, a sixth reagent solution that comprises a sixth solute for detection of TriHexCer 34:1, and a seventh reagent solution that comprises a seventh solute for detection of HexCer 40:0.

In another embodiment, such a kit further comprises: a reagent solution that comprises a first solute for detection of CAV-1; a second solute for detection of SM(40:2); a third solute for detection of SM(44:2); a fourth solute for detection of LacCer 32:0; a fifth solute for detection of LacCer 36:0; a sixth solute for detection of TriHexCer 34:1; and a seventh solute for detection of HexCer 40:0.

In another aspect, the disclosure provides a method of treatment or prevention of progression of prostate cancer in a subject in whom the levels of CAV-1, SM(40:2), SM(44:2), LacCer 32:0, LacCer 36:0, TriHexCer 34:1 and HexCer 40:0 classifies the subject as having or being at risk for progression of prostate cancer comprising one or more of: administering a chemotherapeutic drug to the subject with prostate cancer; administering therapeutic radiation to the subject with prostate cancer; and surgery for partial or complete surgical removal of cancerous tissue in the subject with prostate cancer.

In another aspect, the disclosure provides a method of treatment or prevention of progression of prostate cancer in a subject in whom the levels of CAV-1, SM(40:2), SM(44:2), LacCer 32:0, LacCer 36:0, TriHexCer 34:1 and HexCer 40:0 classifies the subject as having or being at risk for progression of prostate cancer comprising one or more of: administering a chemotherapeutic drug to the subject with prostate cancer; administering therapeutic radiation to the subject with prostate cancer; and surgery for partial or complete surgical removal of cancerous tissue in the subject with prostate cancer.

In another aspect, the disclosure provides a method for treating prostate cancer in a subject, comprising: detecting CAV-1, SM(40:2), SM(44:2), LacCer 32:0, LacCer 36:0, TriHexCer 34:1 and HexCer 40:0, in a biological sample obtained from the subject; quantifying the amounts CAV-1, SM(40:2), SM(44:2), LacCer 32:0, LacCer 36:0, TriHexCer 34:1 and HexCer 40:0 in said collected sample; determining a risk score from the amounts of CAV-1, SM(40:2), SM(44:2), LacCer 32:0, LacCer 36:0, TriHexCer 34:1 and HexCer 40:0; comparing the risk score with a cutoff value to determine whether said human is at risk for progression of prostate cancer; wherein if the levels are above the cutoff value said human is at risk for progression of prostate cancer, and administering a treatment for prostate cancer to said human being at risk for progression of prostate cancer.

In another aspect, the disclosure provides a method of determining risk of a subject for progression of prostate cancer, comprising: in biological samples from a subject in need of analysis, measuring the concentration of CAV-1, SM(40:2), SM(44:2), LacCer 32:0, LacCer 36:0, TriHexCer 34:1 and HexCer 40:0; and comparing the concentration of the biomarkers in the samples of the subject in need of diagnosis and the concentration in a normal or non-diseased subject, wherein the subject in need of diagnosis is diagnosed with prostate cancer.

In another aspect, the disclosure provides a method of determining evidence for risk of progression of prostate cancer in a biological sample, comprising measuring the concentration of a biomarker panel comprising CAV-1, SM(40:2), SM(44:2), LacCer 32:0, LacCer 36:0, TriHexCer 34:1 and HexCer 40:0 in the biological sample, and determining a risk score from the amounts of CAV-1, SM(40:2), SM(44:2), LacCer 32:0, LacCer 36:0, TriHexCer 34:1 and HexCer 40:0. In another embodiment, the levels of CAV-1, SM(40:2), SM(44:2), LacCer 32:0, LacCer 36:0, TriHexCer 34:1 and HexCer 40:0 or the reporter molecules bound thereto are elevated in the subject relative to a healthy subject. In another embodiment, the levels of CAV-1, SM(40:2), SM(44:2), LacCer 32:0, LacCer 36:0, TriHexCer 34:1 and HexCer 40:0 are elevated in comparison to the levels of CAV-1, SM(40:2), SM(44:2), LacCer 32:0, LacCer 36:0, TriHexCer 34:1 and HexCer 40:0 in a reference subject or group that does not have prostate cancer. In another embodiment, the levels of CAV-1, SM(40:2), SM(44:2), LacCer 32:0, LacCer 36:0, TriHexCer 34:1 and HexCer 40:0 are elevated in comparison to the levels of CAV-1, SM(40:2), SM(44:2), LacCer 32:0, LacCer 36:0, TriHexCer 34:1 and HexCer 40:0 in a reference subject or group that has indolent prostate cancer. In another embodiment, the reference subject or group is healthy. In another embodiment, at least one of the surfaces further comprises at least one receptor molecule that selectively binds to a biomarker selected from CAV-1, SM(40:2), SM(44:2), LacCer 32:0, LacCer 36:0, TriHexCer 34:1 and HexCer 40:0. In another embodiment, at least one of the surfaces is the surface of a solid particle. In another embodiment, the solid particle comprises a bead. In another embodiment, such methods comprising: measuring the level of the biomarkers in the biological sample; wherein the amount of the biomarkers classifies the patient as being at risk for progression of prostate cancer or not at risk for progression of prostate cancer. In another embodiment, the sample comprises a biological sample selected from blood, plasma, and serum. In another embodiment, the biological sample is serum. In another embodiment, the amount of CAV-1, SM(40:2), SM(44:2), LacCer 32:0, LacCer 36:0, TriHexCer 34:1 and HexCer 40:0 is quantified. In another embodiment, detection of the amount of CAV-1, SM(40:2), SM(44:2), LacCer 32:0, LacCer 36:0, TriHexCer 34:1 and HexCer 40:0 comprises the use of a solid particle. In another embodiment, the solid particle is a bead. In another embodiment, at least one of the reporter molecules is linked to an enzyme. In another embodiment, at least one of the reporter molecules provides a detectable signal. In another embodiment, the detectable signal is detectable by a method selected from UV-visible spectroscopy, mass spectrometry, nuclear magnetic resonance (NMR) spectroscopy, proton NMR spectroscopy, nuclear magnetic resonance (NMR) spectrometry, gas chromatography-mass spectrometry (GC-MS), liquid chromatography-mass spectrometry (LC-MS), correlation spectroscopy (COSy), nuclear Overhauser effect spectroscopy (NOESY), rotating frame nuclear Overhauser effect spectroscopy (ROESY), LC-TOF-MS, LC-MS/MS, and capillary electrophoresis-mass spectrometry. In another embodiment, the concentrations of CAV-1, SM(40:2), SM(44:2), LacCer 32:0, LacCer 36:0, TriHexCer 34:1 and HexCer 40:0 are measured. In another embodiment, the subject is determined to be at risk for progression of prostate cancer based on the measured concentrations of the biomarkers. In another embodiment, the measured concentrations are used to calculate a biomarker score based on sensitivity and specificity values at a given cutoff. In another embodiment, such methods further comprise the steps of: comparing the measured concentrations of each biomarker in the biological sample to the prediction of a statistical model. In another embodiment, the panel is selected from the group consisting of: a. the panel consisting of CAV-1, SM(40:2), SM(44:2), LacCer 32:0, LacCer 36:0, TriHexCer 34:1 and HexCer 40:0; or b. the panel consisting of CAV-1, SM(40:2), SM(44:2), LacCer 32:0, LacCer 36:0, TriHexCer 34:1 and HexCer 40:0. In another embodiment, the panel comprises biomarkers that have been identified by a method selected from UV-visible spectroscopy, mass spectrometry, nuclear magnetic resonance (NMR) spectroscopy, proton NMR spectroscopy, nuclear magnetic resonance (NMR) spectrometry, gas chromatography-mass spectrometry (GC-MS), liquid chromatography-mass spectrometry (LC-MS), correlation spectroscopy (COSy), nuclear Overhauser effect spectroscopy (NOESY), rotating frame nuclear Overhauser effect spectroscopy (ROESY), LC-TOF-MS, LC-MS/MS, and capillary electrophoresis-mass spectrometry. In another embodiment, the panel comprises biomarkers that have been identified by UV-visible spectroscopy or proton NMR spectroscopy.

In another embodiment, the first reporter binds selectively to CAV-1. In another embodiment, the second reporter binds selectively to SM(40:2). In another embodiment, the third reporter binds selectively to SM(44:2). In another embodiment, the fourth reporter binds selectively to LacCer 32:0. In another embodiment, the fifth reporter binds selectively to LacCer 36:0. In another embodiment, the sixth reporter binds selectively to TriHexCer 34:1. In another embodiment, the seventh reporter binds selectively to HexCer 40:0.

In another embodiment, determination of CAV-1, SM(40:2), SM(44:2), LacCer 32:0, LacCer 36:0, TriHexCer 34:1 and HexCer 40:0 levels is made at substantially the same time. In another embodiment, determination of CAV-1, SM(40:2), SM(44:2), LacCer 32:0, LacCer 36:0, TriHexCer 34:1 and HexCer 40:0 levels is made in a stepwise manner. In another embodiment, such methods comprise inclusion of subject history information into the assignment of being at risk for progression of prostate cancer or not being at risk for progression of prostate cancer. In another embodiment, such methods comprise administering at least one alternate diagnostic test for a subject assigned as being at risk for progression of prostate cancer.

In another embodiment, the method of treating a subject suspected of being at risk for progression of prostate cancer, comprising analyzing the subject for risk for progression of prostate cancer with a method as described herein; and administering a therapeutically effective amount of a treatment for the cancer. In another embodiment, the treatment is surgery, chemotherapy, immunotherapy, radiation therapy, targeted therapy, or a combination thereof. In another embodiment, the levels of CAV-1, SM(40:2), SM(44:2), LacCer 32:0, LacCer 36:0, TriHexCer 34:1 and HexCer 40:0 are elevated in comparison to the levels of CAV-1, SM(40:2), SM(44:2), LacCer 32:0, LacCer 36:0, TriHexCer 34:1 and HexCer 40:0 in a reference subject or group that has adenocarcinoma. In another embodiment, the levels of CAV-1, SM(40:2), SM(44:2), LacCer 32:0, LacCer 36:0, TriHexCer 34:1 and HexCer 40:0 are elevated in comparison to the levels of CAV-1, SM(40:2), SM(44:2), LacCer 32:0, LacCer 36:0, TriHexCer 34:1 and HexCer 40:0 in a reference subject or group that has squamous cell cancer. In another embodiment, the prostate cancer is diagnosed at or before the borderline resectable stage. In another embodiment, the prostate cancer is diagnosed at the resectable stage.

In another embodiment, such methods further comprise: providing a surface that binds CAV-1, SM(40:2), SM(44:2), LacCer 32:0, LacCer 36:0, TriHexCer 34:1 and HexCer 40:0; incubating the surface with the biological sample;

measuring the amount of the first reporter molecule that is associated with the surface; measuring the amount of the second reporter molecule that is associated with the surface; measuring the amount of the third reporter molecule that is associated with the surface; measuring the amount of the fourth reporter molecule that is associated with the surface; measuring the amount of the fifth reporter molecule that is associated with the surface; measuring the amount of the sixth reporter molecule that is associated with the surface; measuring the amount of the seventh reporter molecule that is associated with the surface; wherein the amount of the first reporter molecule, the second reporter molecule, the third reporter molecule, the fourth reporter molecule, the fifth reporter molecule, the sixth reporter molecule, and the seventh reporter molecule classifies the subject as being at risk for progression of prostate cancer or not at risk for progression of prostate cancer.

In another embodiment, such a kit comprises a device for contacting the reagent solutions with a biological sample. In another embodiment, such a kit comprises at least one surface with means for binding at least one biomarker. In another embodiment, the at least one biomarker is selected from the group consisting of CAV-1, SM(40:2), SM(44:2), LacCer 32:0, LacCer 36:0, TriHexCer 34:1 and HexCer 40:0.

In another aspect, the disclosure provides a method comprising a) performing the following steps on obtaining a sample from a subject asymptomatic for prostate cancer; b) measuring a panel of markers in the sample, wherein the markers comprise CAV-1, SM(40:2), SM(44:2), LacCer 32:0, LacCer 36:0, TriHexCer 34:1 and HexCer 40:0; c) determining a biomarker score for each marker; d) summing the biomarker scores for each marker to obtain a composite score for each subject, quantifying the risk for the risk for progression of prostate cancer for the subject as a risk score, wherein the composite score is matched to a risk category of a grouping of stratified subject populations, wherein each risk category comprises a multiplier indicating increased likelihood of having the prostate cancer correlated to a range of composite scores as compared to use of a single threshold value, wherein the multiplier is determined from positive predictive scores of retrospective samples; and, e) administering a computerized tomography (CT) scan or other imagine modality to the subject with a quantified risk for the the risk for progression of prostate cancer. In another embodiment, the markers consist of CAV-1, SM(40:2), SM(44:2), LacCer 32:0, LacCer 36:0, TriHexCer 34:1 and HexCer 40:0. In another embodiment, the sample is blood, blood serum, blood plasma, or some part thereof. In another embodiment, the grouping of a stratified subject population, the multiplier indicating increased likelihood of having the cancer and the range of composite scores are determined from retrospective clinical samples of a population. In another embodiment, the risk category further comprises a risk identifier. In another embodiment, the risk identifier is selected from low risk, intermediate-low risk, intermediate risk, intermediate-high risk and highest risk. In another embodiment, calculating the multiplier indicating increased likelihood of having the cancer for each risk category comprises stratifying the subject cohort based on retrospective biomarker scores and weighting a known prevalence of the cancer in the cohort by a positive predictive score for each stratified population. In another embodiment, the grouping of a stratified subject population comprises at least three risk categories wherein the multiplier indicating increased likelihood of having cancer is about 2 or greater. In another embodiment, the grouping of a stratified subject population comprises at least two risk categories wherein the multiplier indicating increased likelihood of having cancer is about 5 or greater. In another embodiment, the subject is aged 50 years or older and has a history of smoking tobacco. In another embodiment, such methods further comprise generating a risk categorization table, wherein the panel of markers is measured, a biomarker score for each marker is determined, a composite score is obtained by summing the biomarker scores; determining a threshold value used to divide the composite scores into risk groups and assigning a multiplier to each group indicating the likelihood of an asymptomatic subject having a quantified risk for the progression of cancer. In another embodiment, the groups are in a form selected from an electronic table form, a software application, a computer program, and an excel spreadsheet. In another embodiment, the panel of markers comprise proteins, polypeptides, or metabolites measured in a binding assay. In another embodiment, the panel of markers comprise proteins or polypeptides measured using a flow cytometer.

Provided are methods for identifying the risk for progression of prostate cancer in a subject, the method generally comprising: (a) applying a blood sample obtained from the subject to analysis for four biomarkers: CAV-1, SM(40:2), SM(44:2), LacCer 32:0, LacCer 36:0, TriHexCer 34:1 and HexCer 40:0; (b) quantifying the amount of the four biomarkers present in the blood sample; and (c) applying statistical analysis based on the amount of biomarkers present to determine a biomarker score with respect to corresponding prostate cancer, thereby classifying a subject as either positive for risk of progression of prostate cancer or negative for risk of progression of prostate cancer. Alternatively, provided are methods for identifying the risk for progression of prostate cancer in a subject, the method generally comprising: (a) applying a blood sample obtained from the subject to analysis for four biomarkers: CAV-1, SM(40:2), SM(44:2), LacCer 32:0, LacCer 36:0, TriHexCer 34:1 and HexCer 40:0; (b) quantifying the amount of the four biomarkers present in the blood sample; and (c) applying statistical analysis based on the amount of biomarkers present to determine a biomarker score with respect to corresponding prostate cancer, thereby providing a means for assessing in a subject relative risk prostate cancer progression (e.g., in a non-binary fashion).

The methods presented herein enable the screening of high-risk subjects, such as those with a family history of prostate cancer, or subjects with other risk factors such as obesity, heavy smoking, and possibly diabetes. The logistic regression model disclosed herein can incorporate these factors into its classification method.

As used herein, “prostate cancer status” refers to classification of an individual, subject, or patient as being at risk for progression of prostate cancer or as not being at risk for progression of prostate cancer. In some embodiments, an individual being at risk for progression of prostate cancer may be referred to as “prostate cancer-positive.” In other embodiments, an individual not being at risk for progression of prostate cancer may be referred to as “prostate cancer-negative.” For subjects that are classified as prostate cancer-positive, further methods can be provided to clarify prostate cancer status. Classification as prostate cancer-positive can be followed by methods including, but not limited to, computed tomography (CT).

The disclosure is not limited to the specific biomolecules that are reported herein for detection of the biomarkers. Other molecules may be chosen for use in other embodiments, including, but not limited to, biomolecules based on proteins, antibodies, nucleic acids, aptamers, and synthetic organic compounds. Other molecules may demonstrate advantages in terms of sensitivity, efficiency, speed of assay, cost, safety, or ease of manufacture or storage.

In some embodiments, levels of CAV-1, SM(40:2), SM(44:2), LacCer 32:0, LacCer 36:0, TriHexCer 34:1 and HexCer 40:0 in a biological sample are measured. In some embodiments, CAV-1, SM(40:2), SM(44:2), LacCer 32:0, LacCer 36:0, TriHexCer 34:1 and HexCer 40:0 are contacted with reporter molecules, and the levels of respective reporter molecules are measured. In some embodiments, four reporter molecules are provided which specifically bind CAV-1, SM(40:2), SM(44:2), LacCer 32:0, LacCer 36:0, TriHexCer 34:1 and HexCer 40:0, respectively. Use of reporter molecules can provide gains in convenience and sensitivity for the assay.

In some embodiments, CAV-1, SM(40:2), SM(44:2), LacCer 32:0, LacCer 36:0, TriHexCer 34:1 and HexCer 40:0 are adsorbed onto a surface that is provided in a kit. In some embodiments, reporter molecules bind to surface-adsorbed CAV-1, SM(40:2), SM(44:2), LacCer 32:0, LacCer 36:0, TriHexCer 34:1 and HexCer 40:0. Adsorption of biomarkers can be nonselective or selective. In some embodiments, the surface comprises a receptor functionality for increasing selectivity towards adsorption of one or more biomarkers.

In some embodiments, CAV-1, SM(40:2), SM(44:2), LacCer 32:0, LacCer 36:0, TriHexCer 34:1 and HexCer 40:0 are adsorbed onto four surfaces that are selective for one or more of the biomarkers. A reporter molecule or multiple reporter molecules can then bind to surface-adsorbed biomarkers, and the level of reporter molecule(s) associated with a particular surface can allow facile quantification of the particular biomarker that is present on that surface.

In some embodiments, CAV-1, SM(40:2), SM(44:2), LacCer 32:0, LacCer 36:0, TriHexCer 34:1 and HexCer 40:0 are adsorbed onto a surface that is provided in a kit; relay molecules that are specific for one or more of these biomarkers bind to surface-adsorbed biomarkers; and receptor molecules that are specific for one or more relay molecules bind to relay molecules. Relay molecules can provide specificity for certain biomarkers, and receptor molecules can enable detection.

In some embodiments, four relay molecules are provided which specifically bind CAV-1, SM(40:2), SM(44:2), LacCer 32:0, LacCer 36:0, TriHexCer 34:1 and HexCer 40:0, respectively. Relay molecules can be intentionally designed for specificity towards a biomarker, or can be selected from a pool of candidates due to their binding properties.

In some embodiments, CAV-1, SM(40:2), SM(44:2), LacCer 32:0, LacCer 36:0, TriHexCer 34:1 and HexCer 40:0 are adsorbed onto four discrete surfaces that are provided in a kit; relay molecules that are specific for one or more of these biomarkers bind to surface-adsorbed biomarkers; and receptor molecules bind to relay molecules. Analysis of the surfaces can be accomplished in a stepwise or concurrent fashion.

In some embodiments, the reporter molecule is linked to an enzyme, facilitating quantification of reporter molecule. In some embodiments, quantification can be achieved by catalytic production of a substance with desirable spectroscopic properties.

In some embodiments, the amount of biomarker is determined with spectroscopy. In some embodiments, the spectroscopy that is utilized is UV-visible spectroscopy. In some embodiments, the spectroscopy that is utilized is mass spectrometry. In other embodiments, the spectroscopy that is utilized is nuclear magnetic resonance (NMR) spectroscopy, such as including, but not limited to, proton NMR spectroscopy, nuclear magnetic resonance (NMR) spectrometry, gas chromatography-mass spectrometry (GC-MS), liquid chromatography-mass spectrometry (LC-MS), correlation spectroscopy (COSy), nuclear Overhauser effect spectroscopy (NOESY), rotating frame nuclear Overhauser effect spectroscopy (ROESY), LC-TOF-MS, LC-MS/MS, and capillary electrophoresis-mass spectrometry.

The quantity of biomarker or biomarkers that is found in a particular assay can be directly reported to an operator, or alternately it can be stored digitally and readily made available for mathematical processing. A system can be provided for performing mathematical analysis, and can further report classification as prostate cancer-positive or prostate cancer-negative to an operator.

In some embodiments, additional assays known to those of ordinary skill in the art can function with the disclosure herein. Other assays include, but are not limited to, assays utilizing mass-spectrometry, immunoaffinity LC-MS/MS, surface plasmon resonance, chromatography, electrochemistry, acoustic waves, immunohistochemistry and array technologies.

The various system components discussed herein may include one or more of the following: a computer comprising one or more processors for processing digital data; short- or long-term digital memory; an input analog-to-digital converter for providing digitized data; an application program made available to the processor for directing processing of digital data by the processor; an input device for collecting information from the subject or operator, and an output device for displaying information to the subject or operator.

Also provided herein are methods of treatment for subjects who are classified as prostate cancer-positive. Treatment for prostate cancer-positive patients can include, but is not limited to, surgery, chemotherapy, radiation therapy, targeted therapy, or a combination thereof.

With regard to the detection of the biomarkers detailed herein, the disclosure is not limited to the specific biomolecules reported herein. In some embodiments, other biomolecules can be chosen for the detection and analysis of the disclosed biomarkers including, but not limited to, biomolecules based on proteins, antibodies, nucleic acids, aptamers, and synthetic organic compounds. Other molecules may demonstrate advantages in terms of sensitivity, efficiency, speed of assay, cost, safety, or ease of manufacture or storage. In this regard, those of ordinary skill in the art will appreciate that the predicative and diagnostic power of the biomarkers disclosed herein may extend to the analysis of not just the protein form of these biomarkers, but other representations of the biomarkers as well (e.g., nucleic acid). Further, those of ordinary skill in the art will appreciate that the predicative and diagnostic power of the biomarkers disclosed herein can also be used in combination with an analysis of other biomarkers associated with prostate cancer. In some embodiments, other biomarkers associated with prostate cancer can be protein-based biomarkers.

The foregoing has outlined rather broadly the features and technical benefits of the disclosure in order that the detailed description may be better understood. It should be appreciated by those skilled in the art that the specific embodiments disclosed may be readily utilized as a basis for modifying or designing other structures or processes for carrying out the same purposes of the disclosure. It is to be understood that the present disclosure is not limited to the particular embodiments described, as variations of the particular embodiments may be made and still fall within the scope of the appended claims.

### Definitions

As used herein, the term “prostate cancer” refers to a malignant neoplasm of the prostate characterized by the abnormal proliferation of cells, the growth of which cells exceeds and is uncoordinated with that of the normal tissues around it.

As used herein, the term “prostate cancer-positive” refers to classification of a subject as being at risk for progression of prostate cancer.

As used herein, the term “prostate cancer-negative” refers to classification of a subject as not being at risk for progression of prostate cancer.

As used herein, the terms “subject” or “patient” refer to a mammal, preferably a human, for whom a classification as prostate cancer-positive or prostate cancer-negative is desired, and for whom further treatment can be provided.

As used herein, a “reference patient,” “reference subject,” or “reference group” refers to a group of patients or subjects to which a test sample from a patient or subject suspected of having or being at risk for progression of prostate cancer may be compared. In some embodiments, such a comparison may be used to determine whether the test subject has prostate cancer. A reference patient or group may serve as a control for testing or diagnostic purposes. As described herein, a reference patient or group may be a sample obtained from a single patient, or may represent a group of samples, such as a pooled group of samples.

As used herein, “healthy” refers to an individual in whom no evidence of prostate cancer is found, i.e., the individual does not have prostate cancer. Such an individual may be classified as “prostate cancer-negative” or as having a healthy prostate gland, or normal, non-compromised prostate function. A healthy patient or subject has no symptoms of prostate cancer or other prostate disease. In some embodiments, a healthy patient or subject may be used as a reference patient for comparison to diseased or suspected diseased samples for determination of prostate cancer in a patient or a group of patients.

As used herein, the terms “treatment” or “treating” refer to the administration of medicine or the performance of medical procedures with respect to a subject, for either prophylaxis (prevention) or to cure or reduce the extent of or likelihood of occurrence or recurrence of the infirmity or malady or condition or event in the instance where the subject or patient is afflicted. As related to the present disclosure, the term may also mean the administration of pharmacological substances or formulations, or the performance of non-pharmacological methods including, but not limited to, radiation therapy and surgery. Pharmacological substances as used herein may include, but are not limited to, chemotherapeutics that are established in the art, such as abiraterone acetate (Zytiga), apalutamide (Erleada), bicalutamide (Casodex), cabazitaxel (Jevtana), darolutamide (Nubega), degarelix (Firmagon), docetaxel (Taxotere), Eligard (leuprolide acetate), enzalutamide (Xtandi), flutamide, goserelin acetate (Zoladex), leuprolide acetate (Lupron or Lupron Depot), olaparib (Lynparza), mitoxantrone hydrochloride, nilutamide (Nilandron), sipuleucel-T (Provenge), radium 223 dichloride (Xofigo), and rucaparib camsylate (Rubraca).

Pharmacological substances may include substances used in immunotherapy, such as checkpoint inhibitors. Treatment may include a multiplicity of pharmacological substances, or a multiplicity of treatment methods, including, but not limited to, surgery and chemotherapy.

As used herein, the term “ELISA” refers to enzyme-linked immunosorbent assay. This assay generally involves contacting a fluorescently tagged sample of proteins with antibodies having specific affinity for those proteins. Detection of these proteins can be accomplished with a variety of means, including, but not limited to, laser fluorimetry.

As used herein, the term “regression” refers to a statistical method that can assign a predictive value for an underlying characteristic of a sample based on an observable trait (or set of observable traits) of said sample. In some embodiments, the characteristic is not directly observable. For example, the regression methods used herein can link a qualitative or quantitative outcome of a particular biomarker test, or set of biomarker tests, on a certain subject, to a probability that said subject is for prostate cancer-positive.

As used herein, the term “logistic regression” refers to a regression method in which the assignment of a prediction from the model can have one of several allowed discrete values. For example, the logistic regression models used herein can assign a prediction, for a certain subject, of either prostate cancer-positive or prostate cancer-negative.

As used herein, the term “biomarker score” refers to a numerical score for a particular subject that is calculated by inputting the particular biomarker levels for said subject to a statistical method.

As used herein, the term “composite score” refers to a summation of the normalized values for the predetermined markers measured in the sample from the subject. In one embodiment, the normalized values are reported as a biomarker score and those biomarker score values are then summed to provide a composite score for each subjected tested. When used in the context of the risk categorization table and correlated to a stratified grouping based on a range of composite scores in the Risk Categorization Table, the “composite score” is used to determine the “risk score” for each subject tested wherein the multiplier indicating increased likelihood of having the cancer for the stratified grouping becomes the “risk score,”

As used herein, the term “risk score” refers to a single numerical value that indicates an asymptomatic human subject's risk for progression of cancer cancer as compared to the known prevalence of cancer progression in the disease cohort. In certain embodiments, the composite score as calculated for a human subject and correlated to a multiplier indicating risk for progression of prostate cancer, wherein the composite score is correlated based on the range of composite scores for each stratified grouping in the risk categorization table. In this way the composite score is converted to a risk score based on the multiplier indicating increased likelihood of having the cancer for the grouping that is the best match for the composite score.

As used herein, the term “cutoff” or “cutoff point” refers to a mathematical value associated with a specific statistical method that can be used to assign a classification of prostate cancer-positive of prostate cancer-negative to a subject, based on said subject's biomarker score.

As used herein, when a numerical value above or below a cutoff value “is characteristic of prostate cancer,” what is meant is that the subject, analysis of whose sample yielded the value, either has prostate cancer or is at risk for progression of prostate cancer.

As used herein, a subject who is “risk for progression of prostate cancer” is one who may not yet evidence overt symptoms of prostate cancer, or whose prostate cancer is currently indolent, but who is producing levels of biomarkers which indicate that the subject has prostate cancer, or may develop it in the near term. A subject who has prostate cancer or is suspected of harboring prostate cancer may be treated for the cancer or suspected cancer.

As used herein, the term “classification” refers to the assignment of a subject as being at risk for progression of prostate cancer or not being at risk for progression of prostate cancer, based on the result of the biomarker score that is obtained for said subject.

As used herein, the term “Wilcoxon rank sum test,” also known as the Mann-Whitney U test, Mann-Whitney-Wilcoxon test, or Wilcoxon-Mann-Whitney test, refers to a specific statistical method used for comparison of two populations. For example, the test can be used herein to link an observable trait, in particular a biomarker level, to the absence or the risk for progression of prostate cancer in subjects of a certain population.

As used herein, the term “true positive rate” refers to the probability that a given subject classified as positive by a certain method is truly positive.

As used herein, the term “false positive rate” refers to the probability that a given subject classified as positive by a certain method is truly negative.

As used herein, the term “sensitivity” refers to, in the context of various biochemical assays, the ability of an assay to correctly identify those with a disease (i.e., the true positive rate). By comparison, as used herein, the term “specificity” refers to, in the context of various biochemical assays, the ability of an assay to correctly identify those without the disease (i.e., the true negative rate). Sensitivity and specificity are statistical measures of the performance of a binary classification test (i.e., classification function). Sensitivity quantifies the avoiding of false negatives, and specificity does the same for false positives.

As used herein, a “sample” refers to a test substance to be tested for the presence of, and levels or concentrations thereof, of a biomarker as described herein. A sample may be any substance appropriate in accordance with the present disclosure, including, but not limited to, blood, blood serum, blood plasma, or any part thereof.

As used herein, a “metabolite” refers to small molecules that are intermediates and/or products of cellular metabolism. Metabolites may perform a variety of functions in a cell, for example, structural, signaling, stimulatory and/or inhibitory effects on enzymes. In some embodiments, a metabolite may be a non-protein, plasma-derived metabolite marker, such as including, but not limited to, acetylspermidine, diacetylspermine, lysophosphatidylcholine (18:0), lysophosphatidylcholine (20:3), and an indole-derivative.

As used herein, the term “ROC” refers to receiver operating characteristic, which is a graphical plot used herein to gauge the performance of a certain diagnostic method at various cutoff points. A ROC plot can be constructed from the fraction of true positives and false positives at various cutoff points.

As used herein, the term “AUC” refers to the area under the curve of the ROC plot. AUC can be used to estimate the predictive power of a certain diagnostic test. Generally, a larger AUC corresponds to increasing predictive power, with decreasing frequency of prediction errors. Possible values of AUC range from 0.5 to 1.0, with the latter value being characteristic of an error-free prediction method.

As used herein, the term “p-value” or “p” refers to the probability that the distributions of biomarker scores for prostate cancer-positive and prostate cancer-negative subjects are identical in the context of a Wilcoxon rank sum test. Generally, a p-value close to zero indicates that a particular statistical method will have high predictive power in classifying a subject.

As used herein, the term “CI” refers to a confidence interval, i.e., an interval in which a certain value can be predicted to lie with a certain level of confidence. As used herein, the term “95% CI” refers to an interval in which a certain value can be predicted to lie with a 95% level of confidence.

As used herein, the term “disease progression” or “early disease progression” is defined as upgrading of Gleason score and/or increased tumor volume on surveillance biopsy within 18 months after start of active surveillance.

As used herein, the term “indolent disease” or “indolent” prostate cancer is defined as the absence of progression for five or more years after start of active surveillance.

### Abbreviations

AKT=RAC serine/threonine-protein kinase; AS=active surveillance; AUC=area under the curve; Cav-1=caveolin-1; CCLE=Broad Institute Cancer Cell Line Encyclopedia; CE=cholesterol ester; CM=conditioned media; DiI=1,1′-dioctadecyl-3,3,3′,3′-tetramethylindocarbocyanine; DP=disease progression; FC=free cholesterol; GS=Gleason score; HexCer=hexosylceramide; HexCer 40:0=hexosylceramide(40:0); HR=hazard ratio; LacCer=lactosylceramide; LacCer 32:0=lactosylceramide(32:0); LacCer 36:0=lactosylceramide(36:0); MAPK=MAP-kinase=mitogen-activated protein kinase; PC=phosphatidylcholine; PDMP=1-phenyl-2-decanoylamino-3-morpholino-1-propanol; PPMP=D-threo-1-phenyl-2-hexadecanoylamino-3-morpholino-1-propanol; PI3K=phosphoinositide 3-kinase=phosphatidylinositol-3-kinase; RFU=relative fluorescence unit; ROC=receiver operating characteristic; SEM=standard error of the mean: SFM=serum-free media; sHDL=synthetic HDL-like particles; sLDL=synthetic LDL-like particles; SM=sphingomyelin; SSALP=synthetic self-assembled lipid particle; TCGA=The Cancer Genome Atlas; TO=trioleate; TriHexCer=trihexosylceramide; TriHexCer 34:1=trihexosylceramide(34:1).

### EXAMPLES

The following examples are included to demonstrate embodiments of the disclosure. The following examples are presented only by way of illustration and to assist one of ordinary skill in using the disclosure. The examples are not intended in any way to otherwise limit the scope of the disclosure. Those of skill in the art should, in light of the present disclosure, appreciate that many changes can be made in the specific embodiments which are disclosed and still obtain a like or similar result without departing from the spirit and scope of the disclosure.

### Example 1: Isolation of Extracellular Vesicles by Density Gradient Flotation

Extracellular vesicles were isolated as previously described. Briefly, microvesicles were depleted from biospecimen samples by centrifugation at 2000×g for 20 min followed by 16,500×g for 30 min; resulting supernatant was filtered through a pre-wetted 0.22 μm vacuum filter. Microvesicle-depleted biospecimen was densified by mixing with OptiPrep iodixanol solution (Sigma D1556) to a final density of 1.16-1.30 g mL⁻¹ and loaded into the bottom of a polycarbonate ultracentrifuge tube and overlaid with 0.5-2 mL aliquots of iodixanol/PBS solution in the 1.20-1.01 g mL⁻¹ (35-0% wt:vol) range, proceeding from the highest to lowest density to form a single- or multi-step density fractionation gradient as needed. Ultracentrifugation was performed for at 100,000×g for 4 hrs at 8° C. Vesicles were collected from the top of the tube, proceeding downward to recover volume equal to 90% of overlaid gradient volume. Density of harvested fractions was assessed against a standard curve based on sample absorbance at 250 nm using a NanoDrop microvolume spectrophotometer (ThermoFisher Scientific, Wilmington, DE). Vesicle harvests were stored at −80° C.

### Example 2: Proteomic Profiling of Extracellular Vesicles

Proteomic profiling of extracellular vesicles was performed according to the following standardized workflows. Briefly, ECV-derived protein digestion and identification by LC-MS/MS was performed using established protocols. NanoAcquity UPLC system coupled in-line with WATERS SYNAPT G2-Si mass spectrometer was used for the separation of pooled digested protein fractions. The system was equipped with a Waters Symmetry C18 nanoAcquity trap-column (180 μm×20 mm, 5 μm) and a Waters HSS-T3 C18 nanoAcquity analytical column (75 μm×150 mm, 1.8 μm). The column oven temperature was set at 50° C., and the temperature of the tray compartment in the auto-sampler was set at 6° C. LC-HDMSE data were acquired in resolution mode with SYNAPT G2-Si using Waters Masslynx (version 4.1, SCN 851). The capillary voltage was set to 2.80 kV, sampling cone voltage to 30 V. source offset to 30 V and source temperature to 100° C. Mobility utilized high-purity N2 as the drift gas in the IMS TriWave cell. Pressures in the helium cell, Trap cell, IMS TriWave cell and Transfer cell were 4.50 mbar, 2.47×10⁻², 2.90. and 2.53×10⁻³ mbar, respectively. IMS wave velocity was 600 n s⁻¹, helium cell DC 50 V, Trap DC bias 45 V, IMS TiWave DC bias V and IMS wave delay 1000 μs. The mass spectrometer was operated in V-mode with a typical resolving power of at least 20,000. All analyses were performed using positive mode ESI using a NanoLockSpray source. The lock mass channel was sampled every 60 s. The mass spectrometer was calibrated with a [Glu1] fibrinopeptide solution (300 fmol μL⁻¹) delivered through the reference sprayer of the NanoLockSpray source. Accurate mass LC-HDMSE data were collected in an alternating, low energy (MS) and high energy (MSE) mode of acquisition with mass scan range from m z⁻¹ 50 to 1800. The spectral acquisition time in each mode was 1.0 s with a 0.1-s inter-scan delay. In low energy HDMS mode, data were collected at constant collision energy of 2 eV in both Trap cell and Transfer cell. In high-energy HDMSE mode, the collision energy was ramped from 25 to 55 eV in the Transfer cell only. The RF applied to the quadrupole mass analyzer was adjusted such that ions from m z⁻¹ 300 to 2000 were efficiently transmitted, ensuring that any ions observed in the LC-HDMSE data<m z⁻¹ of 300 arose from dissociations in the Transfer collision cell. The acquired LC-HDMSE data were processed and searched against protein knowledge database (Uniprot) through ProteinLynx Global Server (PLGS, Waters Company) with 4% FDR.

### Example 3: Metabolomics Analyses

Sample Extraction Following transfections, cell lysates were washed 2× with pre-chilled 0.9% NaCl followed by addition of 2.5 mL of pre-chilled 3:1 isopropanol:ultrapure water. Cells were scraped using a 25 cm Cell Scraper (Sarstedt) in extraction solvent and transferred to a 15 mL conical tube (Eppendorf). Samples were briefly vortexed followed by centrifugation at 4° C. for 10 min at 2,000×g. Thereafter, 1.2 mL of metabolite extracts were transferred to 1.5 mL Eppendorf tubes and stored in −20° C. until metabolomic analysis.

Primary Metabolites and Biogenic Amines Plasma metabolites were extracted from pre-aliquoted EDTA plasma (10 μL) with 30 μL of LCMS grade methanol (ThermoFisher) in a 96-well microplate (Eppendorf). Plates were heat sealed, vortexed for 5 min at 750 rpm, and centrifuged at 2000×g for 10 minutes at room temperature. The supernatant (10 μL) was carefully transferred to a 96-well plate, leaving behind the precipitated protein. The supernatant was further diluted with 10 μL of 100 mM ammonium formate, pH 3. For Hydrophilic Interaction Liquid Chromatography (HILIC) analysis, the samples were diluted with 60 μL LCMS grade acetonitrile (ThermoFisher), whereas samples for C18 analysis were diluted with 60 μL water (GenPure ultrapure water system, ThermoFisher). Each sample solution was transferred to 384-well microplate (Eppendorf) for LCMS analysis. For conditioned media, frozen samples were thawed on ice and 30 μl transferred to a 96-well microplate (Eppendorf). Aliquots were diluted with an additional 30 μL of 100 mM ammonium formate. Microplates were heat sealed, vortexed for 5 min at 1500 rpm, and centrifuged at 2000×g for 10 minutes at room temperature. For Hydrophilic Interaction Liquid Chromatography (HILIC) analysis, the 25 μL of sample was transferred to a new 96 well microplate containing 75 μL acetonitrile, whereas samples for C18 analysis were transferred to a new 96-well microplate containing 75 μL water (GenPure ultrapure water system, ThermoFisher). Each sample solution was transferred to 384-well microplate (Eppendorf) for LCMS analysis. Cell lysate supernatant, 100 μL (3:1 isopropanol:ultrapure water) was aliquoted into two 96-well plates (Eppendorf) and evaporated to dryness under vacuum. The samples were then reconstituted as follows: for the HILIC assays, the dried samples were dissolved in 65 μL of ACN (ThermoFisher): 100 mM Ammonium Formate pH3 (9:1) whereas for the C18 reverse phase assays, the dried samples were dissolved in 65 μL of H₂O: 100 mM Ammonium Formate pH3 (9:1). The samples were spun down to remove any insoluble materials and then transferred to a 384-well plate for high throughput mass analysis using LCMS.

Complex Lipids Pre-aliquoted EDTA plasma samples (10 μL) were extracted with 30 μL of LCMS grade 2-propanol (ThermoFisher) in a 96-well microplate (Eppendorf). Plates were heat sealed, vortexed for 5 min at 750 rpm, and centrifuged at 2000×g for 10 minutes at room temperature. The supernatant (10 μL) was carefully transferred to a 96-well plate, leaving behind the precipitated protein. The supernatant was further diluted with 90 μL of 1:3:2 100 mM ammonium formate, pH 3 (ThermoFisher): acetonitrile: 2-propanol and transferred to a 384-well microplate (Eppendorf) for lipids analysis using LCMS. For cell lysates, in a 96 well plate, 10 μL (3:1 isopropanol:ultrapure water) of the cell lysates supernatant was diluted with 90 μL of 1:3:2 100 mM ammonium formate, pH 3:acetonitrile:2-propanol (ThermoFisher) and transferred to a 384-well microplate (Eppendorf) for analysis by LC-MS.

Untargeted Analysis of Primary Metabolites and Biogenic Amines

Untargeted metabolomics analysis was conducted on Waters Acquity™ UPLC system with 2D column regeneration configuration (I-class and H-class) coupled to a Xevo G2-XS quadrupole time-of-flight (qTOF) mass spectrometer. Chromatographic separation was performed using HILIC (Acquity™ UPLC BEH amide, 100 Å, 1.7 μm 2.1×100 mm, Waters Corporation, Milford, U.S.A) and C18 (Acquity™ UPLC HSS T3, 100 Å, 1.8 μm, 2.1×100 mm, Water Corporation, Milford, U.S.A) columns at 45° C. Quaternary solvent system mobile phases were (A) 0.1% formic acid in water, (B) 0.1% formic acid in acetonitrile and (D) 100 mM ammonium formate, pH 3. Samples were separated using the following gradient profile: for the HILIC separation a starting gradient of 95% B and 5% D was increase linearly to 70% A, 25% B and 5% D over a 5 min period at 0.4 mL min⁻¹ flow rate, followed by 1 min isocratic gradient at 100% A at 0.4 mL min⁻¹ flow rate. For C18 separation, a chromatography gradient of was as follows: starting conditions, 100% A, with linear increase to final conditions of 5% A, 95% B followed by isocratic gradient at 95% B, 5% D for 1 min. Binary pump was used for column regeneration and equilibration. The solvent system mobile phases were (A1) 100 mM ammonium formate, pH 3, (A2) 0.1% formic in 2-propanol and (B1) 0.1% formic acid in acetonitrile. The HILIC column was stripped using 90% A2 for 5 min followed by 2 min equilibration using 100% B1 at 0.3 mL min⁻¹ flowrate. Reverse phase C18 column regeneration was performed using 95% A1, 5% B1 for 2 min followed by column equilibration using 5% A1, 95% B1 for 5 min.

Untargeted Analysis of Complex Lipids For the lipidomic assay, untargeted metabolomics analysis was conducted on a Waters Acquity™ UPLC system coupled to a Xevo G2-XS quadrupole time-of-flight (qTOF) mass spectrometer. Chromatographic separation was performed using a C18 (Acquity™ UPLC HSS T3, 100 Å, 1.8 μm, 2.1×100 mm, Water Corporation, Milford, USA) column at 55° C. The mobile phases were (A) water, (B) Acetonitrile, (C) 2-propanol and (D) 500 mM ammonium formate, pH 3. A starting elution gradient of 20% A, 30% B, 49% C and 1% D was increased linearly to 10% B, 89% C and 1% D for 5.5 min, followed by isocratic elution at 10% B, 89% C and 1% D for 1.5 min and column equilibration with initial conditions for 1 min.

### Example 4: Mass Spectrometry

Data Acquisition. Mass spectrometry data was acquired using sensitivity mode in positive and negative electrospray ionization mode within 50-1200 Da range for primary metabolites and 100-2000 Da for complex lipids. For the electrospray acquisition, the capillary voltage was set at 1.5 kV (positive), 3.0 kV (negative), sample cone voltage 30 V, source temperature at 120° C., cone gas flow 50 L h⁻¹ and desolvation gas flow rate of 800 L h⁻¹ with scan time of 0.5 sec in continuum mode. Leucine Enkephalin; 556.2771 Da (positive) and 554.2615 Da (negative) was used for lockspray correction and scans were performed at 0.5 min. The injection volume for each sample was 3 μL, unless otherwise specified. The acquisition was carried out with instrument auto gain control to optimize instrument sensitivity over the samples acquisition time.

Data Processing LC-MS and LC-MSe data were processed using Progenesis QI (Nonlinear, Waters) and values were reported as area units. Annotations were determined by matching accurate mass and retention times using customized libraries created from authentic standards and/or by matching experimental tandem mass spectrometry data against the NIST MSMS or HMDB v3 theoretical fragmentations.

Data Normalization To correct for injection order drift, each feature was normalized using data from repeat injections of quality control samples collected every 10 injections throughout the run sequence. Measurement data were smoothed by Locally Weighted Scatterplot Smoothing (LOESS) signal correction (QC-RLSC) as previously described. Feature values between quality control samples were interpolated by a cubic spline. Metabolite values were rescaled by using the overall median of the historical quality control peak areas across all samples. Only detected features exhibiting a relative standard deviation (RSD) less than 30 in either the historical or pooled quality controls samples were considered for further statistical analysis. To reduce data matrix complexity, annotated features with multiple adducts or acquisition mode repeats were collapsed to one representative unique feature. Features were selected based on replicate precision (RSD<30), highest intensity and best isotope similarity matching to theoretical isotope distributions. Values are reports as ratios relative to the historical quality control reference samples that is included in every analytical run (plasma/conditioned media) or adjusted area units (lysates).

Statistical Analyses In order to find the cut-off point for the covariate that gives the largest difference between individuals in the two already defined groups, the previously described method was employed. Using log rank statistic-based on the groups defined by cut-off affords:

$${S}_{k} = {\sum }_{i = 1}^{D} [ {d}_{i}^{+} - {d}_{i} \frac{{r}_{i}^{+}}{{r}_{i}} ]$$

where D is the total number of distinct events (disease progression (DP)), dᵢ is the total number of DP at each event time (tᵢ), dᵢ⁺ is the total number of DP when the Cav-1-sphingolipid signature value is bigger than the cut-off point. rᵢ and rᵢ⁺ also define as the total number at risk for all Cav-1-sphingolipid signature values and Cav-1-sphingolipid signature values larger than cut-off point, respectively. Sₖ was calculated for all possible cut point in Cav-1-sphingolipid signature column and the estimated cut point is the value that yields the maximum Sₖ. In this analysis, the maximum value of Sₖ is at the top 16.4% of Cav-1-sphingolipid signature values. In another word, the top 16.4% of Cav-1-sphingolipid signature values are in the high-risk group and the other 83.6% are in the low-risk group.

In order to calculate the p-value of this test use of the following formula provided the value of 0.009. It suggests that the Cav-1-sphingolipid signature level highly relates to progression free survival.

$$p - value \approx 2 exp ( - 2 {Q}^{2} ) \\ where : \\ Q = \frac{max \vert {S}_{k} \vert}{s \sqrt{D - 1}} \\ and \\ {s}^{2} = \frac{1}{D - 1} {\sum }_{i = 1}^{D} {\{ 1 - {\sum }_{j = 1}^{i} \frac{1}{D - j + 1} \}}^{2}$$

### Example 5: Prediction of AS Gleason Grade Progression by Plasma Lipid Signature

Untargeted metabolomics analyses were conducted on clinically matched baseline plasma samples (n=16 per group) prospectively collected from patients with clinically low-risk early stage prostate cancer undergoing AS who exhibited early DP or indolent disease (Table 1). A total of 269 unique annotated metabolite features were identified in baseline plasmas from the discovery cohort; 14 features exhibited statistically significant (unadjusted Wilcoxon-rank sum test p-value<0.05) ROC AUC values>0.7.

TABLE 1
Patient characteristics for MDACC discovery cohort.

| | Total | Cases | Controls | p |
|---|---|---|---|---|
| N | 32 | 16 | 16 | |
| Age, y | 64.4 ± 7.9 | 64.3 ± 8.2 | 64.4 ± 7.9 | 0.9548 |
| BMI, kg/m² | 27.0 ± 3.9 | 27.3 ± 3.5 | 26.7 ± 4.3 | 0.7219 |
| Family history of PC | | | | |
| 1st degree | 8 (25) | 3 (18.8) | 5 (31.3) | 0.6851 |
| 2nd degree | 4 (12.5) | 3 (18.8) | 1 (6.3) | 0.5996 |
| Hypertension | 13 (40.6) | 5 (31.3) | 8 (50) | 0.4725 |
| Diabetes | 5 (15.6) | 3 (18.8) | 2 (12.5) | 1 |
| Smoking (ever smoker) | 14 (43.8) | 8 (50) | 6 (37.5) | 0.4757 |
| 5ARI | 3 (9.4) | 0 (0) | 3 (18.8) | 0.2258 |
| Statins | 10 (31.3) | 4 (25) | 6 (37.5) | 0.7043 |
| PSA | 3.8 ± 2.1 | 3.7 ± 1.8 | 3.9 ± 2.3 | 0.9849 |
| Testosterone | 383.7 ± 179.2 | 400.9 ± 197.0 | 366.5 ± 164.1 | 0.6922 |
| TRUS (Total) | 42.0 ± 18.1 | 41.8 ± 19.6 | 42.2 ± 17.3 | 0.771 |
| TRUS (TZ) | 18.7 ± 11.0 | 19.2 ± 11.3 | 18.3 ± 11.0 | 0.8264 |
| Summation Total Tumor Length (mm) | 4.6 ± 4.9 | 4.8 ± 3.9 | 4.5 ± 5.9 | 0.4249 |
| Highest Gleason Score | 1 | | | |
| 3 + 3 | 24 (75) | 12 (75) | 12 (75) | |
| 3 + 4 | 7 (21.9) | 3 (18.8) | 4 (25) | |
| 4 + 3 | 1 (3.1) | 1 (6.3) | 0 (0) | |

Seven of the 14 features were complex lipids; in particular, sphingomyelins and glycosphingolipids (Table 2).

TABLE 2
Metabolite features identified in the discovery cohort

| Accepted Compound ID | Domain | AUC1 | p-value# | AUC2 | p-value# | AUC3 | p-value# |
|---|---|---|---|---|---|---|---|
| 1-Methylhistidine | (a) | 0.41 | 0.381 | 0.50 | 1.000 | 0.52 | 0.867 |
| 1-Methylnicotinamide | (a) | 0.37 | 0.224 | 0.45 | 0.669 | 0.54 | 0.724 |
| 2-aminobenzamide | (a) | 0.43 | 0.515 | 0.45 | 0.642 | 0.61 | 0.287 |
| 3-Methoxytyramine | (a) | 0.46 | 0.752 | 0.39 | 0.287 | 0.36 | 0.184 |
| 3-Methylhistamine | (a) | 0.38 | 0.239 | 0.52 | 0.897 | 0.54 | 0.752 |
| 3-Methylhistidine | (a) | 0.32 | 0.094 | 0.45 | 0.616 | 0.38 | 0.254 |
| 4-Acetamido-2- | (a) | 0.59 | 0.423 | 0.50 | 0.985 | 0.59 | 0.423 |
| aminobutanoic acid | | | | | | | |
| 4-Hydroxy-L-proline | (a) | 0.57 | 0.491 | 0.37 | 0.210 | 0.48 | 0.897 |
| 4-phosphopantothenoyl- | (a) | 0.40 | 0.361 | 0.52 | 0.867 | 0.49 | 0.926 |
| cysteine | | | | | | | |
| 5-Oxo-D-proline | (a) | 0.52 | 0.867 | 0.55 | 0.669 | 0.65 | 0.149 |
| α-N-Phenylacetyl-L- | (a) | 0.49 | 0.956 | 0.42 | 0.445 | 0.48 | 0.867 |
| glutamine | | | | | | | |
| Aniline | (a) | 0.45 | 0.616 | 0.42 | 0.468 | 0.48 | 0.867 |
| Citrulline | (a) | 0.36 | 0.184 | 0.62 | 0.270 | 0.53 | 0.780 |
| Creatine | (a) | 0.50 | 0.985 | 0.54 | 0.696 | 0.50 | 0.985 |
| Creatinine | (a) | 0.39 | 0.305 | 0.46 | 0.724 | 0.45 | 0.669 |
| D-Aspartate | (a) | 0.59 | 0.381 | 0.41 | 0.423 | 0.50 | 0.985 |
| Diethanolamine | (a) | 0.56 | 0.564 | 0.46 | 0.752 | 0.48 | 0.838 |
| DL-5-Hydroxylysine | (a) | 0.45 | 0.616 | 0.54 | 0.696 | 0.55 | 0.669 |
| D-Ornithine | (a) | 0.31 | 0.073 | 0.47 | 0.809 | 0.48 | 0.897 |
| Glutathione | (a) | 0.46 | 0.752 | 0.61 | 0.305 | 0.51 | 0.956 |
| Homoserine | (a) | 0.55 | 0.616 | 0.62 | 0.254 | 0.67 | 0.110 |
| L-Arginine | (a) | 0.45 | 0.642 | 0.52 | 0.838 | 0.49 | 0.956 |
| L-Asparagine | (a) | 0.45 | 0.616 | 0.40 | 0.341 | 0.52 | 0.897 |
| L-Cystathionine | (a) | 0.55 | 0.616 | 0.46 | 0.696 | 0.51 | 0.956 |
| L-Cystine | (a) | 0.46 | 0.752 | 0.47 | 0.809 | 0.46 | 0.752 |
| Leucine | (a) | 0.58 | 0.468 | 0.61 | 0.323 | 0.62 | 0.270 |
| L-Glutamine | (a) | 0.52 | 0.897 | 0.60 | 0.341 | 0.58 | 0.468 |
| L-Histidine | (a) | 0.67 | 0.110 | 0.63 | 0.224 | 0.65 | 0.160 |
| L-Histidinol | (a) | 0.34 | 0.119 | 0.45 | 0.616 | 0.38 | 0.254 |
| L-Isoleucine | (a) | 0.51 | 0.926 | 0.64 | 0.184 | 0.61 | 0.287 |
| L-Kynurenine | (a) | 0.32 | 0.086 | 0.46 | 0.752 | 0.49 | 0.956 |
| L,L-2.6-Diamino- | (a) | 0.65 | 0.160 | 0.48 | 0.897 | 0.68 | 0.086 |
| heptanedioate | | | | | | | |
| L-Lysine | (a) | 0.40 | 0.361 | 0.55 | 0.616 | 0.60 | 0.341 |
| L-Methionine | (a) | 0.43 | 0.515 | 0.61 | 0.305 | 0.61 | 0.323 |
| L-N^{γ}.-Monomethylarginine | (a) | 0.57 | 0.515 | 0.63 | 0.239 | 0.63 | 0.224 |
| L-Phenylalanine | (a) | 0.55 | 0.669 | 0.46 | 0.752 | 0.67 | 0.110 |
| L-Pipecolic Acid | (a) | 0.61 | 0.305 | 0.57 | 0.539 | 0.59 | 0.381 |
| L-Proline | (a) | 0.52 | 0.897 | 0.66 | 0.138 | 0.65 | 0.149 |
| L-Serine | (a) | 0.46 | 0.752 | 0.34 | 0.119 | 0.51 | 0.926 |
| L-Threonine | (a) | 0.51 | 0.956 | 0.46 | 0.724 | 0.56 | 0.590 |
| L-Tryptophan | (a) | 0.56 | 0.590 | 0.45 | 0.616 | 0.67 | 0.102 |
| L-Tyrosine | (a) | 0.52 | 0.897 | 0.55 | 0.616 | 0.61 | 0.305 |
| L-Valine | (a) | 0.56 | 0.590 | 0.51 | 0.926 | 0.56 | 0.564 |
| N(Pai)-Methyl-L-Histidine | (a) | 0.33 | 0.102 | 0.52 | 0.867 | 0.45 | 0.669 |
| N⁸-Acetylspermidine | (a) | 0.53 | 0.809 | 0.49 | 0.926 | 0.60 | 0.361 |
| N⁶,N⁶,N⁶-Trimethyllysine | (a) | 0.57 | 0.515 | 0.45 | 0.616 | 0.54 | 0.752 |
| N^{γ},N^{γ}-Dimethyl-L- | (a) | 0.48 | 0.897 | 0.46 | 0.752 | 0.48 | 0.897 |
| Arginine | | | | | | | |
| Nicotinamide | (a) | 0.49 | 0.956 | 0.68 | 0.086 | 0.54 | 0.724 |
| Nicotinamide | (a) | 0.71 | 0.039 | 0.55 | 0.669 | 0.69 | 0.067 |
| Mononucleotide | | | | | | | |
| Nicotinate β-D- | (a) | 0.50 | 0.985 | 0.56 | 0.590 | 0.46 | 0.752 |
| Ribonucleotide | | | | | | | |
| Nicotinuric Acid | (a) | 0.38 | 0.239 | 0.45 | 0.616 | 0.43 | 0.539 |
| N-Methyl-D-Aspartic Acid | (a) | 0.52 | 0.897 | 0.61 | 0.323 | 0.54 | 0.752 |
| N-Methyl-L-Glutamate | (a) | 0.55 | 0.616 | 0.51 | 0.956 | 0.55 | 0.616 |
| N-Methylnicotinamide | (a) | 0.39 | 0.323 | 0.52 | 0.867 | 0.49 | 0.926 |
| N-Undecanoylglycine | (a) | 0.32 | 0.086 | 0.42 | 0.468 | 0.36 | 0.196 |
| O-Acetyl-L-Serine | (a) | 0.38 | 0.270 | 0.36 | 0.184 | 0.34 | 0.119 |
| O-Phospho-L-Serine | (a) | 0.49 | 0.956 | 0.25 | 0.014 | 0.49 | 0.956 |
| Picolinic Acid | (a) | 0.36 | 0.184 | 0.52 | 0.897 | 0.48 | 0.838 |
| Pyroglutamic Acid | (a) | 0.66 | 0.119 | 0.62 | 0.254 | 0.67 | 0.110 |
| Quinoline | (a) | 0.52 | 0.838 | 0.46 | 0.752 | 0.60 | 0.361 |
| Thyroxine | (a) | 0.69 | 0.067 | 0.63 | 0.210 | 0.76 | 0.011 |
| Tyramine | (a) | 0.45 | 0.616 | 0.51 | 0.926 | 0.51 | 0.926 |
| Urocanate | (a) | 0.36 | 0.184 | 0.36 | 0.184 | 0.36 | 0.171 |
| 6-Phosphogluconic Acid | (b) | 0.38 | 0.270 | 0.54 | 0.724 | 0.34 | 0.128 |
| α-D-Glucose | (b) | 0.61 | 0.305 | 0.53 | 0.809 | 0.57 | 0.539 |
| D-Galactose | (b) | 0.73 | 0.026 | 0.57 | 0.539 | 0.72 | 0.035 |
| D-Glucose-6-Phosphate | (b) | 0.35 | 0.160 | 0.49 | 0.956 | 0.29 | 0.047 |
| Galactitol | (b) | 0.63 | 0.239 | 0.44 | 0.590 | 0.48 | 0.897 |
| Maltose | (b) | 0.48 | 0.867 | 0.41 | 0.381 | 0.51 | 0.956 |
| Melibiose | (b) | 0.53 | 0.809 | 0.58 | 0.468 | 0.60 | 0.341 |
| N-Acetyllactosamine | (b) | 0.41 | 0.423 | 0.39 | 0.323 | 0.50 | 0.985 |
| N-Acetylneuraminate | (b) | 0.36 | 0.184 | 0.39 | 0.323 | 0.39 | 0.287 |
| Acetylcarnitine | (c) | 0.53 | 0.780 | 0.47 | 0.809 | 0.43 | 0.515 |
| Acylcarnitine(C10:0) | (c) | 0.50 | 0.985 | 0.41 | 0.402 | 0.44 | 0.590 |
| Acylcarnitine(C14:0) | (c) | 0.50 | 0.985 | 0.41 | 0.402 | 0.53 | 0.809 |
| Acylcarnitine(C16:0) | (c) | 0.46 | 0.752 | 0.58 | 0.468 | 0.54 | 0.696 |
| Acylcarnitine(C18:0) | (c) | 0.48 | 0.838 | 0.53 | 0.809 | 0.48 | 0.897 |
| Acylcarnitine(C18:1) | (c) | 0.53 | 0.809 | 0.60 | 0.341 | 0.49 | 0.956 |
| Deoxycarnitine | (c) | 0.63 | 0.239 | 0.55 | 0.616 | 0.58 | 0.468 |
| Dodecanedioylcarnitine | (c) | 0.45 | 0.616 | 0.45 | 0.616 | 0.44 | 0.590 |
| Glutarylcarnitine | (c) | 0.47 | 0.809 | 0.47 | 0.780 | 0.44 | 0.590 |
| Hexanoylcarnitine | (c) | 0.63 | 0.224 | 0.51 | 0.956 | 0.53 | 0.809 |
| Hydroxybutyrylcarnitine | (c) | 0.39 | 0.287 | 0.44 | 0.564 | 0.34 | 0.119 |
| L-Carnitine | (c) | 0.51 | 0.926 | 0.41 | 0.423 | 0.45 | 0.642 |
| O-Acetyl-L-Carnitine | (c) | 0.57 | 0.491 | 0.47 | 0.809 | 0.48 | 0.897 |
| Octanoylcarnitine | (c) | 0.55 | 0.642 | 0.44 | 0.590 | 0.50 | 1.000 |
| Propionylcarnitine | (c) | 0.61 | 0.287 | 0.54 | 0.724 | 0.57 | 0.539 |
| Tiglylcarnitine | (c) | 0.49 | 0.926 | 0.51 | 0.956 | 0.47 | 0.809 |
| Cer(41:1) | (d) | 0.55 | 0.616 | 0.52 | 0.867 | 0.59 | 0.402 |
| Aspartyl-threonine | (e) | 0.55 | 0.642 | 0.43 | 0.515 | 0.52 | 0.867 |
| Glycyl-threonine | (e) | 0.42 | 0.468 | 0.64 | 0.184 | 0.58 | 0.468 |
| Histidinyl-proline | (e) | 0.54 | 0.724 | 0.56 | 0.564 | 0.75 | 0.015 |
| 1-(Hydroxymethyl)-5,5- | (f) | 0.54 | 0.696 | 0.63 | 0.239 | 0.65 | 0.160 |
| dimethyl-2,4- | | | | | | | |
| imidazolidinedione | | | | | | | |
| Exogenous | | | | | | | |
| 13-Nor-6-eremophilene- | (f) | 0.40 | 0.341 | 0.44 | 0.590 | 0.39 | 0.287 |
| 8,11-dione Exogenous | | | | | | | |
| Benzofuran | (f) | 0.52 | 0.867 | 0.56 | 0.590 | 0.62 | 0.254 |
| cis-Quinceoxepane | (f) | 0.58 | 0.445 | 0.50 | 0.985 | 0.50 | 0.985 |
| Metformin | (f) | 0.53 | 0.780 | 0.52 | 0.897 | 0.47 | 0.809 |
| Trazodone | (f) | 0.55 | 0.669 | 0.30 | 0.051 | 0.39 | 0.323 |
| 4,7-Dioxo-octanoic Acid | (g) | 0.59 | 0.423 | 0.35 | 0.149 | 0.61 | 0.323 |
| cis-9, cis-12- | (g) | 0.65 | 0.149 | 0.46 | 0.696 | 0.59 | 0.423 |
| Octadecadienoic Acid | | | | | | | |
| Dodecanoic acid | (g) | 0.64 | 0.184 | 0.54 | 0.696 | 0.57 | 0.515 |
| Heptanoic acid | (g) | 0.54 | 0.724 | 0.51 | 0.956 | 0.53 | 0.780 |
| Linoleate | (g) | 0.62 | 0.254 | 0.48 | 0.838 | 0.52 | 0.838 |
| Myristoleic acid | (g) | 0.54 | 0.752 | 0.38 | 0.270 | 0.64 | 0.184 |
| GlcCer(39:2) | (h) | 0.74 | 0.019 | 0.77 | 0.008 | 0.82 | 0.001 |
| LacCer(32:0) | (h) | 0.74 | 0.019 | 0.76 | 0.012 | 0.82 | 0.001 |
| LacCer(32:1) | (h) | 0.69 | 0.067 | 0.69 | 0.073 | 0.78 | 0.007 |
| LacCer(34:1) | (h) | 0.64 | 0.171 | 0.67 | 0.102 | 0.68 | 0.094 |
| Neuac?2-3Gal?1-4Glc?- | (h) | 0.49 | 0.956 | 0.53 | 0.809 | 0.51 | 0.926 |
| Cer(D18:1/16:0) | | | | | | | |
| [Trihexosylcer- | | | | | | | |
| (D18:1/16:0)] | | | | | | | |
| Neuac?2-3Gal?-Cer(34:1) | (h) | 0.62 | 0.254 | 0.60 | 0.361 | 0.66 | 0.138 |
| [Dihexosylcer(34:1)] | | | | | | | |
| Neuac?2-3Gal?-Cer(36:1) | (h) | 0.62 | 0.270 | 0.60 | 0.341 | 0.66 | 0.119 |
| [Dihexosylcer(36:1)] | | | | | | | |
| Trihexosylceramide(34:1) | (h) | 0.64 | 0.171 | 0.55 | 0.642 | 0.55 | 0.642 |
| Corticosterone | (i) | 0.65 | 0.149 | 0.48 | 0.897 | 0.64 | 0.196 |
| Cortisol | (i) | 0.54 | 0.724 | 0.39 | 0.305 | 0.74 | 0.021 |
| Cortisol 21-Acetate | (i) | 0.54 | 0.724 | 0.37 | 0.224 | 0.57 | 0.539 |
| Cortisone | (i) | 0.55 | 0.669 | 0.48 | 0.867 | 0.64 | 0.184 |
| Noradrenaline | (i) | 0.35 | 0.160 | 0.43 | 0.539 | 0.50 | 0.985 |
| Putative_Progesterone 3- | (i) | 0.71 | 0.043 | 0.73 | 0.023 | 0.79 | 0.005 |
| Biotin | | | | | | | |
| Serotonin | (i) | 0.52 | 0.838 | 0.47 | 0.809 | 0.66 | 0.138 |
| Glycerophosphocholine | (j) | 0.57 | 0.491 | 0.58 | 0.445 | 0.59 | 0.402 |
| Phosphocholine | (j) | 0.50 | 1.000 | 0.64 | 0.196 | 0.52 | 0.838 |
| Sn-Glycerol 3-Phosphate | (j) | 0.50 | 0.985 | 0.57 | 0.515 | 0.52 | 0.897 |
| LPC(14:0) | (k) | 0.58 | 0.468 | 0.50 | 0.985 | 0.59 | 0.423 |
| LPC(15:0) | (k) | 0.54 | 0.752 | 0.56 | 0.590 | 0.58 | 0.468 |
| LPC(16:0) | (k) | 0.55 | 0.616 | 0.51 | 0.926 | 0.61 | 0.323 |
| LPC(16:1) | (k) | 0.54 | 0.696 | 0.52 | 0.838 | 0.54 | 0.696 |
| LPC(17:0) | (k) | 0.61 | 0.287 | 0.48 | 0.867 | 0.62 | 0.254 |
| LPC(17:1) | (k) | 0.54 | 0.752 | 0.52 | 0.897 | 0.54 | 0.724 |
| LPC(18:0) | (k) | 0.60 | 0.341 | 0.55 | 0.642 | 0.63 | 0.224 |
| LPC(18:2) | (k) | 0.50 | 1.000 | 0.51 | 0.956 | 0.53 | 0.780 |
| LPC(18:3) | (k) | 0.47 | 0.780 | 0.44 | 0.564 | 0.52 | 0.897 |
| LPC(20:1) | (k) | 0.54 | 0.724 | 0.57 | 0.491 | 0.49 | 0.926 |
| LPC(20:2) | (k) | 0.56 | 0.564 | 0.57 | 0.539 | 0.52 | 0.838 |
| LPC(20:3) | (k) | 0.50 | 1.000 | 0.54 | 0.752 | 0.47 | 0.809 |
| LPC(20:4) | (k) | 0.44 | 0.590 | 0.52 | 0.838 | 0.42 | 0.468 |
| LPC(20:5) | (k) | 0.55 | 0.642 | 0.44 | 0.564 | 0.54 | 0.724 |
| LPC(22:5) | (k) | 0.57 | 0.515 | 0.58 | 0.445 | 0.53 | 0.809 |
| LPC(22:6) | (k) | 0.51 | 0.926 | 0.50 | 1.000 | 0.52 | 0.897 |
| LPC(26:0) | (k) | 0.61 | 0.323 | 0.61 | 0.287 | 0.74 | 0.019 |
| LPC(P-16:0) | (k) | 0.52 | 0.838 | 0.63 | 0.224 | 0.55 | 0.669 |
| LPC(P-18:0) | (k) | 0.68 | 0.086 | 0.59 | 0.381 | 0.62 | 0.254 |
| LPC(P-18:0/0:0) or LPC(O- | (k) | 0.62 | 0.254 | 0.61 | 0.305 | 0.58 | 0.468 |
| 18:1) | | | | | | | |
| LPE(18:1) | (l) | 0.43 | 0.539 | 0.48 | 0.867 | 0.46 | 0.696 |
| LPE(18:2) | (l) | 0.45 | 0.669 | 0.54 | 0.696 | 0.45 | 0.669 |
| LPE(19:0) | (l) | 0.66 | 0.128 | 0.61 | 0.287 | 0.61 | 0.305 |
| LPE(22:0) | (l) | 0.65 | 0.149 | 0.63 | 0.239 | 0.68 | 0.080 |
| LPE(22:6) | (l) | 0.62 | 0.254 | 0.49 | 0.956 | 0.54 | 0.696 |
| 1-Linoleoylglycerol | (m) | 0.67 | 0.102 | 0.60 | 0.361 | 0.69 | 0.067 |
| Monoelaidin | (m) | 0.57 | 0.515 | 0.55 | 0.642 | 0.55 | 0.642 |
| 1-Methyladenosine | (n) | 0.53 | 0.780 | 0.44 | 0.590 | 0.54 | 0.752 |
| 5,6-Dihydrouridine | (n) | 0.43 | 0.491 | 0.44 | 0.590 | 0.41 | 0.402 |
| Adenosine 3′.5′-diphosphate | (n) | 0.49 | 0.956 | 0.56 | 0.564 | 0.43 | 0.515 |
| Adenosine 5′- | (n) | 0.52 | 0.897 | 0.57 | 0.539 | 0.50 | 1.000 |
| monophosphate | | | | | | | |
| Alloxan | (n) | 0.57 | 0.491 | 0.66 | 0.119 | 0.69 | 0.067 |
| Guanosine 3′.5′-cyclic | (n) | 0.68 | 0.080 | 0.59 | 0.423 | 0.64 | 0.171 |
| Monophosphate | | | | | | | |
| Hypoxanthine | (n) | 0.63 | 0.210 | 0.55 | 0.642 | 0.71 | 0.043 |
| Paraxanthine | (n) | 0.66 | 0.119 | 0.62 | 0.254 | 0.79 | 0.005 |
| UDP-GlcNAc | (n) | 0.41 | 0.423 | 0.52 | 0.838 | 0.43 | 0.539 |
| Uridine | (n) | 0.71 | 0.043 | 0.60 | 0.361 | 0.68 | 0.086 |
| Xanthine | (n) | 0.48 | 0.867 | 0.52 | 0.897 | 0.57 | 0.515 |
| Xanthurenic Acid | (n) | 0.60 | 0.361 | 0.63 | 0.239 | 0.66 | 0.119 |
| 1-Hydroxy-2-Naphthoate | (o) | 0.43 | 0.539 | 0.47 | 0.780 | 0.48 | 0.867 |
| 2-Hydroxy-4-(methylthio)- | (o) | 0.50 | 1.000 | 0.41 | 0.381 | 0.43 | 0.515 |
| butyric acid | | | | | | | |
| 3-(4-Hydroxyphenyl)- | (o) | 0.57 | 0.491 | 0.50 | 0.985 | 0.53 | 0.780 |
| propionic acid | | | | | | | |
| 4-Hydroxy-2-quinoline- | (o) | 0.73 | 0.026 | 0.57 | 0.515 | 0.62 | 0.270 |
| carboxylic acid | | | | | | | |
| Citrate | (o) | 0.57 | 0.491 | 0.67 | 0.102 | 0.59 | 0.402 |
| Coumaric acid | (o) | 0.50 | 0.985 | 0.55 | 0.616 | 0.58 | 0.445 |
| Hydroxyphenanthrene | (o) | 0.71 | 0.043 | 0.54 | 0.752 | 0.73 | 0.029 |
| Indole-3-lactic acid | (o) | 0.56 | 0.590 | 0.54 | 0.696 | 0.51 | 0.926 |
| Indoleacrylic acid | (o) | 0.51 | 0.926 | 0.47 | 0.809 | 0.68 | 0.094 |
| Pyrrole-2-carboxylic acid | (o) | 0.44 | 0.590 | 0.45 | 0.642 | 0.45 | 0.642 |
| Urate | (o) | 0.48 | 0.838 | 0.51 | 0.956 | 0.49 | 0.926 |
| Betaine | (p) | 0.54 | 0.752 | 0.49 | 0.956 | 0.45 | 0.669 |
| Biliverdin | (p) | 0.71 | 0.047 | 0.67 | 0.102 | 0.66 | 0.119 |
| Caffeine | (p) | 0.57 | 0.539 | 0.61 | 0.305 | 0.65 | 0.160 |
| Dethiobiotin | (p) | 0.57 | 0.491 | 0.53 | 0.780 | 0.66 | 0.128 |
| Indole-3-acetaldehyde | (p) | 0.57 | 0.515 | 0.47 | 0.780 | 0.66 | 0.128 |
| Indoleacetaldehyde | (p) | 0.52 | 0.838 | 0.57 | 0.491 | 0.56 | 0.590 |
| Piperine | (p) | 0.45 | 0.669 | 0.51 | 0.926 | 0.51 | 0.956 |
| PC(30:0) | (q) | 0.43 | 0.515 | 0.47 | 0.809 | 0.53 | 0.780 |
| PC(31:0) | (q) | 0.50 | 0.985 | 0.46 | 0.724 | 0.49 | 0.926 |
| PC(32:1) | (q) | 0.39 | 0.305 | 0.45 | 0.642 | 0.46 | 0.752 |
| PC(32:2) | (q) | 0.56 | 0.590 | 0.68 | 0.094 | 0.61 | 0.323 |
| PC(34:1) | (q) | 0.62 | 0.270 | 0.35 | 0.149 | 0.48 | 0.867 |
| PC(34:2) | (q) | 0.70 | 0.061 | 0.43 | 0.539 | 0.54 | 0.724 |
| PC(35:1) | (q) | 0.66 | 0.119 | 0.45 | 0.669 | 0.61 | 0.287 |
| PC(36:2) | (q) | 0.73 | 0.026 | 0.47 | 0.780 | 0.73 | 0.023 |
| PC(36:3) | (q) | 0.56 | 0.564 | 0.52 | 0.838 | 0.65 | 0.149 |
| PC(36:4) | (q) | 0.57 | 0.491 | 0.38 | 0.270 | 0.57 | 0.539 |
| PC(36:5) | (q) | 0.55 | 0.642 | 0.42 | 0.468 | 0.56 | 0.564 |
| PC(38:5) | (q) | 0.55 | 0.669 | 0.46 | 0.724 | 0.55 | 0.616 |
| PC(38:6) | (q) | 0.54 | 0.752 | 0.58 | 0.445 | 0.62 | 0.254 |
| PC(38:7) | (q) | 0.54 | 0.724 | 0.49 | 0.926 | 0.56 | 0.590 |
| PC(40:7) | (q) | 0.61 | 0.323 | 0.60 | 0.341 | 0.61 | 0.287 |
| PC(o-30:1) or PC(p-30:0) | (q) | 0.56 | 0.564 | 0.46 | 0.724 | 0.59 | 0.381 |
| PC(o-32:1) or PC(p-32:0) | (q) | 0.41 | 0.423 | 0.53 | 0.809 | 0.53 | 0.809 |
| PC(o-34:4) or PC(p-34:3) | (q) | 0.50 | 0.985 | 0.47 | 0.809 | 0.52 | 0.867 |
| PC(o-38:4) or PC(p-38:3) | (q) | 0.54 | 0.724 | 0.57 | 0.515 | 0.63 | 0.239 |
| PC(o-38:6) or PC(p-38:6) | (q) | 0.56 | 0.590 | 0.59 | 0.423 | 0.55 | 0.616 |
| PC(o-40:7) or PC(p-40:6) | (q) | 0.56 | 0.590 | 0.51 | 0.956 | 0.60 | 0.361 |
| PE(36:1) | (r) | 0.65 | 0.149 | 0.58 | 0.445 | 0.70 | 0.056 |
| PE(36:2) | (r) | 0.57 | 0.539 | 0.64 | 0.196 | 0.68 | 0.086 |
| PE(36:4) | (r) | 0.44 | 0.590 | 0.45 | 0.669 | 0.48 | 0.867 |
| PE(37:4) | (r) | 0.65 | 0.149 | 0.45 | 0.669 | 0.56 | 0.590 |
| PE(38:3) | (r) | 0.69 | 0.073 | 0.60 | 0.361 | 0.73 | 0.023 |
| PE(38:3) | (r) | 0.58 | 0.445 | 0.58 | 0.445 | 0.66 | 0.119 |
| PE(40:6) | (r) | 0.56 | 0.590 | 0.41 | 0.423 | 0.52 | 0.867 |
| PE(o-36:5) or PE(p-36:4) | (r) | 0.64 | 0.171 | 0.64 | 0.184 | 0.70 | 0.051 |
| PE(p-18:0_20:4) | (r) | 0.60 | 0.361 | 0.65 | 0.160 | 0.71 | 0.039 |
| PI(38:4) | (s) | 0.66 | 0.119 | 0.67 | 0.110 | 0.74 | 0.021 |
| Putative_PI(36:2) | (s) | 0.72 | 0.032 | 0.76 | 0.012 | 0.76 | 0.012 |
| Putative_PI(40:3) | (s) | 0.65 | 0.149 | 0.70 | 0.056 | 0.78 | 0.006 |
| Leukotriene B4 | (t) | 0.46 | 0.696 | 0.46 | 0.752 | 0.39 | 0.305 |
| Prostaglandin A1 | (t) | 0.75 | 0.014 | 0.55 | 0.642 | 0.64 | 0.184 |
| Prostaglandin E2 | (t) | 0.55 | 0.616 | 0.36 | 0.184 | 0.52 | 0.867 |
| SM(32:1) | (u) | 0.63 | 0.224 | 0.57 | 0.539 | 0.62 | 0.270 |
| SM(32:2) | (u) | 0.65 | 0.149 | 0.64 | 0.196 | 0.63 | 0.239 |
| SM(34:1) | (u) | 0.50 | 0.985 | 0.59 | 0.423 | 0.55 | 0.616 |
| SM(34:2) | (u) | 0.56 | 0.590 | 0.61 | 0.287 | 0.58 | 0.468 |
| SM(36:1) | (u) | 0.63 | 0.239 | 0.61 | 0.323 | 0.65 | 0.149 |
| SM(36:2) | (u) | 0.48 | 0.867 | 0.53 | 0.780 | 0.55 | 0.642 |
| SM(40:2) | (u) | 0.73 | 0.029 | 0.65 | 0.160 | 0.72 | 0.032 |
| SM(42:1) | (u) | 0.52 | 0.838 | 0.52 | 0.867 | 0.53 | 0.809 |
| SM(42:3) | (u) | 0.74 | 0.019 | 0.64 | 0.171 | 0.71 | 0.039 |
| 7-oxo-cholesterol | (v) | 0.61 | 0.305 | 0.48 | 0.897 | 0.50 | 0.985 |
| 8-Deoxy-11,13- | (v) | 0.44 | 0.564 | 0.53 | 0.809 | 0.35 | 0.149 |
| dihydroxygrosheimin | | | | | | | |
| Cholate | (v) | 0.66 | 0.119 | 0.51 | 0.926 | 0.58 | 0.445 |
| Deoxycholate | (v) | 0.55 | 0.642 | 0.53 | 0.780 | 0.54 | 0.724 |
| Glycocholate | (v) | 0.55 | 0.616 | 0.50 | 0.985 | 0.52 | 0.838 |
| Sterol | (v) | 0.62 | 0.254 | 0.55 | 0.669 | 0.60 | 0.361 |
| TG(16:0_18:1_18:2) | (w) | 0.50 | 0.985 | 0.51 | 0.956 | 0.47 | 0.809 |
| TG(44:0) | (w) | 0.58 | 0.468 | 0.47 | 0.809 | 0.54 | 0.696 |
| TG(45:0) | (w) | 0.61 | 0.323 | 0.47 | 0.780 | 0.59 | 0.423 |
| TG(46:0) | (w) | 0.62 | 0.254 | 0.48 | 0.897 | 0.64 | 0.184 |
| TG(46:1) | (w) | 0.58 | 0.468 | 0.53 | 0.809 | 0.55 | 0.669 |
| TG(48:1) | (w) | 0.55 | 0.642 | 0.49 | 0.926 | 0.56 | 0.564 |
| TG(48:2) | (w) | 0.57 | 0.539 | 0.53 | 0.780 | 0.55 | 0.642 |
| TG(49:0) | (w) | 0.59 | 0.381 | 0.44 | 0.590 | 0.55 | 0.616 |
| TG(49:1) | (w) | 0.54 | 0.696 | 0.41 | 0.381 | 0.55 | 0.616 |
| TG(50:2) | (w) | 0.47 | 0.780 | 0.51 | 0.926 | 0.50 | 1.000 |
| TG(50:3) | (w) | 0.55 | 0.669 | 0.54 | 0.752 | 0.53 | 0.809 |
| TG(51:3) | (w) | 0.56 | 0.590 | 0.55 | 0.669 | 0.54 | 0.696 |
| TG(51:4) | (w) | 0.57 | 0.515 | 0.54 | 0.724 | 0.57 | 0.491 |
| TG(52:0) | (w) | 0.54 | 0.752 | 0.50 | 1.000 | 0.53 | 0.780 |
| TG(52:1) | (w) | 0.46 | 0.724 | 0.46 | 0.752 | 0.50 | 1.000 |
| TG(52:4) | (w) | 0.50 | 0.985 | 0.54 | 0.752 | 0.52 | 0.897 |
| TG(52:7) | (w) | 0.61 | 0.323 | 0.47 | 0.780 | 0.59 | 0.423 |
| TG(53:1) | (w) | 0.57 | 0.515 | 0.43 | 0.539 | 0.59 | 0.423 |
| TG(53:2) | (w) | 0.54 | 0.724 | 0.46 | 0.696 | 0.51 | 0.926 |
| TG(54:1) | (w) | 0.59 | 0.381 | 0.51 | 0.956 | 0.54 | 0.724 |
| TG(54:2) | (w) | 0.43 | 0.515 | 0.52 | 0.897 | 0.44 | 0.564 |
| TG(54:3) | (w) | 0.40 | 0.361 | 0.49 | 0.956 | 0.46 | 0.724 |
| TG(54:5) | (w) | 0.43 | 0.539 | 0.52 | 0.838 | 0.50 | 0.985 |
| TG(54:6) | (w) | 0.47 | 0.809 | 0.48 | 0.897 | 0.52 | 0.897 |
| TG(55:2) | (w) | 0.48 | 0.867 | 0.43 | 0.539 | 0.52 | 0.897 |
| TG(56:7) | (w) | 0.54 | 0.752 | 0.52 | 0.867 | 0.57 | 0.515 |
| TG(57:1) | (w) | 0.44 | 0.564 | 0.48 | 0.838 | 0.50 | 1.000 |
| TG(58:9) | (w) | 0.54 | 0.724 | 0.50 | 0.985 | 0.59 | 0.423 |
| TG(61:13) | (w) | 0.55 | 0.642 | 0.32 | 0.086 | 0.54 | 0.752 |
| TG(65:11) | (w) | 0.43 | 0.515 | 0.44 | 0.590 | 0.57 | 0.491 |
| TG(65:11)_iso | (w) | 0.52 | 0.838 | 0.45 | 0.669 | 0.59 | 0.402 |
| 3-cis-Hydroxy-b,e-caroten- | (x) | 0.68 | 0.080 | 0.49 | 0.956 | 0.66 | 0.128 |
| 3′-one | | | | | | | |
| 4-Pyridoxate | (x) | 0.35 | 0.149 | 0.48 | 0.838 | 0.49 | 0.926 |
| Choline | (x) | 0.51 | 0.956 | 0.46 | 0.724 | 0.49 | 0.956 |
| D-Pantothenic acid | (x) | 0.38 | 0.270 | 0.43 | 0.515 | 0.50 | 0.985 |
| Folic acid | (x) | 0.53 | 0.780 | 0.42 | 0.445 | 0.49 | 0.956 |
| Riboflavin | (x) | 0.62 | 0.254 | 0.54 | 0.724 | 0.55 | 0.669 |

(a) amino acid or amide
(b) carbohydrate
(c) carnitine
(d) ceramide
(e) dipeptide
(f) exogenous
(g) fatty acid
(h) glycosphingolipid
(i) hormone
(j) lipid
(k) lysophosphatidylcholine
(l) lysophosphatidylethanolamine
(m) monoacylglycerol
(n) purine/pyrimidine
(o) organic acid
(p) other
(q) phosphatidylcholine
(r) phosphatidylethanolamine
(s) phosphatidylinositol
(t) prostanoid
(u) sphingomyelin
(v) stereol
(w) triacylglycerol
(x) vitamin

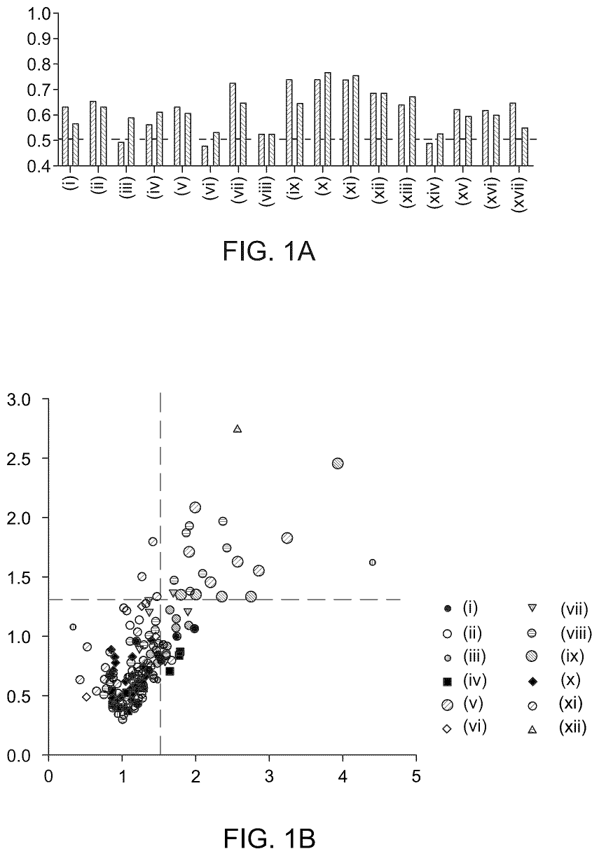

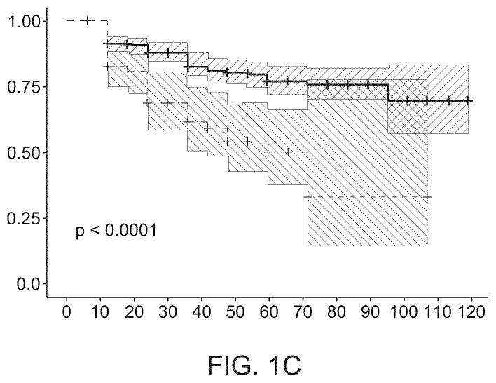

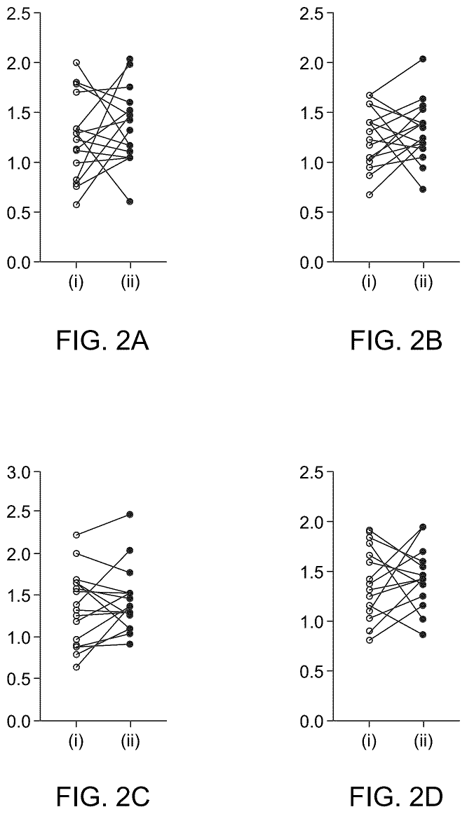

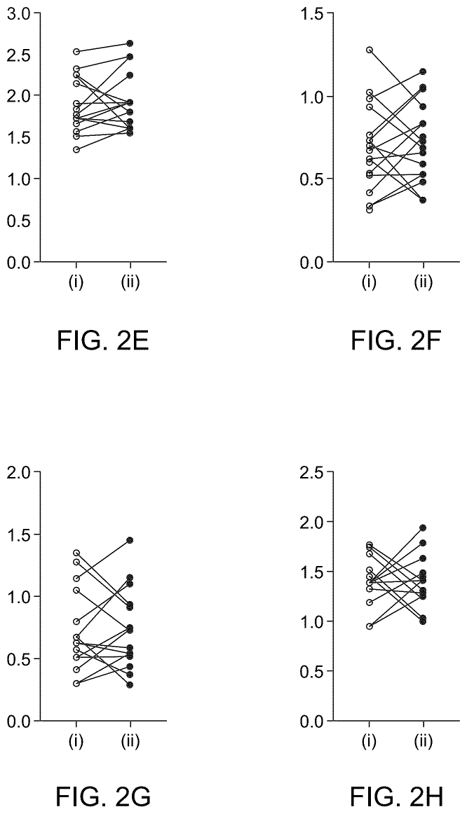

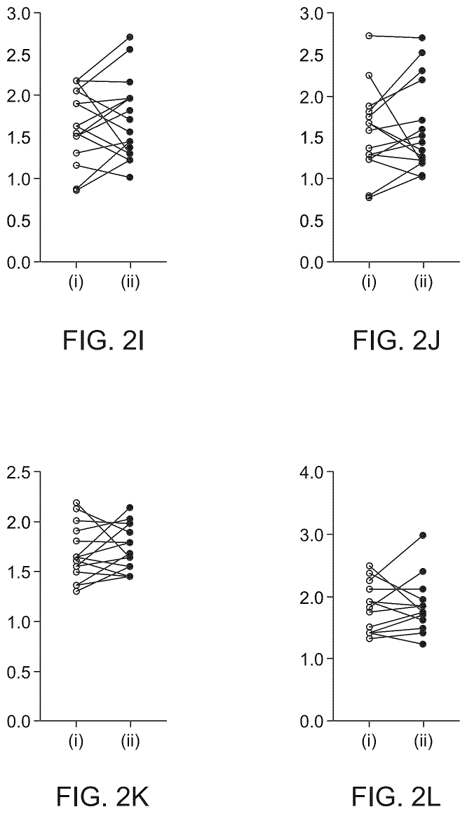

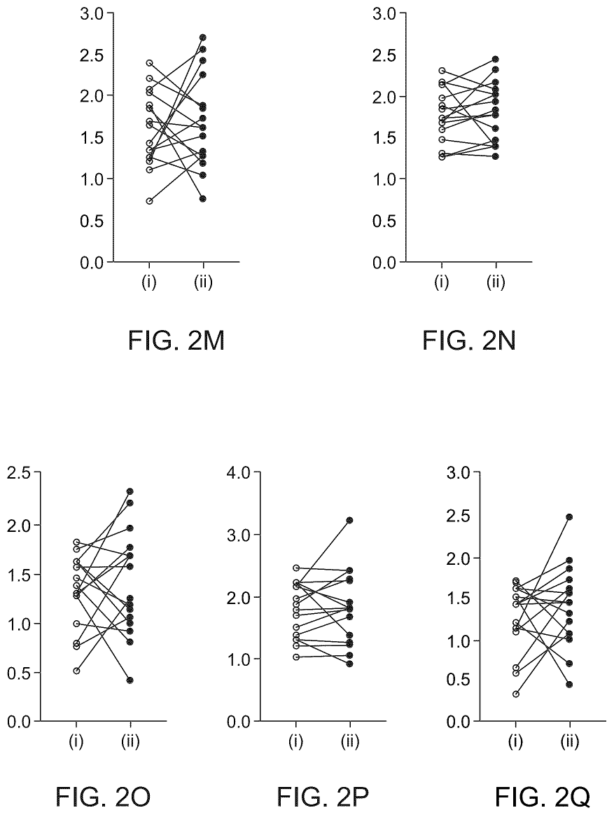

Although none of the 14 features remained individually significant after adjusting for multiple hypothesis testing, the significantly elevated lipids are biochemically linked to ceramide metabolism, suggesting a coordinated signal. Further, sphingomyelins and glycosphingolipids, in general, were elevated in baseline plasma samples of progressor cases (early DP) as compared to controls (no disease progression for a minimum of 5 years after start of AS) (FIG. 1A). Importantly, the detected SMs and glycosphingolipids remained elevated in cases at DP as compared to follow up time-matched controls. Intra-case comparisons of SMs and glycosphingolipids observed at baseline vs. 12 months post-baseline were not statistically significant (FIGS. 2A-2Q). In light of the known lipid transport functions of Cav-1 (Cheng 2016), the observed elevated plasma sphingolipid signature suggested a biological linkage to previous findings of elevated plasma Cav-1 in the context of disease progression. To explore this, analyses were expanded to include untargeted metabolomics profiling on 459 baseline plasma samples prospectively collected from patients with early-stage prostate cancer undergoing AS (Table 3).

TABLE 3
Patient characteristics for MDACC validation cohort

| | Case | Control | P |
|---|---|---|---|
| Subjects, N | 98 | 361 | |
| age, mean ± stdev | 65 ± 7.4 | 63 ± 8.4 | 0.035\hat{ } |
| BMI, mean ± stdev | 29.2 ± 4.53 | 28.9 ± 4.51 | 0.748\hat{ } |
| PSA Density, mean ± stdev | 0.13 ± 0.083 | 0.11 ± 0.103 | 0.002\hat{ } |
| Testosterone, mean ± stdev | 396 ± 161.8 | 396 ± 162.5 | 0.922\hat{ } |
| Smoking, N (%) | | | |
| Yes | 61 (62) | 206 (57) | 0.419\hat{ }\hat{ } |
| No | 37 (38) | 155 (43) | |
| 5-ARI Treatment, N (%) | | | |
| Yes | 7 (7) | 42 (12) | 0.268\hat{ }\hat{ } |
| No | 91 (93) | 319 (88) | |
| Statin Use, N (%) | | | |
| Yes | 52 (53) | 165 (46) | 0.211\hat{ }\hat{ } |
| No | 46 (47) | 196 (54) | |
| Follow-up time (months), | 25 (12-96) | 41.7 (6-120) | |
| mean (range) | | | |

\hat{ }2-sided Wilcoxon-rank Sum Test
\hat{ }\hat{ }2-sided Fisher's Exact Test

Consistent with findings in the initial discovery cohort, multiple SMs and glycosphingolipids were positively associated (Hazard Ratio>1.5) with DP based on GS (FIG. 1; Table 4:).

TABLE 4
Hazard ratios of individual metabolites for progression-
free survival in the MDACC validation cohort

| | | | 2-sided | 1-sided | Lower | Upper |
|---|---|---|---|---|---|---|
| Metabolite | Domain | HR# | p-value^{‡} | p-value | 95 CI | 95 CI |
| Acetylcarnitine | (a) | 1.4 | 0.123 | 0.062 | 0.92 | 2.09 |
| Acylcarnitine(C14:0) | (a) | 1.1 | 0.616 | 0.308 | 0.72 | 1.73 |
| Acylcarnitine(C16:0) | (a) | 1.1 | 0.693 | 0.346 | 0.61 | 2.09 |
| Acylcarnitine(C18:0) | (a) | 1.3 | 0.510 | 0.255 | 0.64 | 2.44 |
| Acylcarnitine(C18:1) | (a) | 1.2 | 0.499 | 0.249 | 0.70 | 2.11 |
| Acylcarnitine(C18:2n-6) | (a) | 1.1 | 0.639 | 0.320 | 0.69 | 1.85 |
| Acylcarnitine(C8:0) | (a) | 1.0 | 0.696 | 0.348 | 0.93 | 1.12 |
| Dodecanedioylcarnitine | (a) | 1.2 | 0.506 | 0.253 | 0.72 | 1.97 |
| Lauroylcarnitine | (a) | 1.1 | 0.298 | 0.149 | 0.89 | 1.46 |
| O-butanoyl-R-carnitine | (a) | 1.2 | 0.599 | 0.299 | 0.68 | 1.95 |
| O-decanoylcarnitine | (a) | 1.0 | 0.487 | 0.243 | 0.93 | 1.17 |
| O-hexanoyl-R-carnitine | (a) | 1.2 | 0.412 | 0.206 | 0.82 | 1.63 |
| O-octanoyl-R-carnitine | (a) | 1.0 | 0.620 | 0.310 | 0.91 | 1.17 |
| Ceramide(18:1_24:1) | (b) | 0.8 | 0.474 | 0.237 | 0.43 | 1.48 |
| Ceramide(18:2_16:0) | (b) | 1.3 | 0.550 | 0.275 | 0.55 | 3.07 |
| Ceramide(30:1) | (b) | 1.7 | 0.396 | 0.198 | 0.52 | 5.21 |
| Ceramide(32:1) | (b) | 0.9 | 0.745 | 0.373 | 0.34 | 2.16 |
| Ceramide(34:1) | (b) | 1.8 | 0.286 | 0.143 | 0.62 | 5.18 |
| Ceramide(39:1) | (b) | 0.9 | 0.819 | 0.410 | 0.52 | 1.68 |
| Ceramide(40:0) | (b) | 0.9 | 0.628 | 0.314 | 0.56 | 1.43 |
| Ceramide(40:1) | (b) | 1.0 | 0.880 | 0.440 | 0.61 | 1.77 |
| Ceramide(40:2) | (b) | 0.9 | 0.622 | 0.311 | 0.51 | 1.50 |
| Ceramide(41:1) | (b) | 1.1 | 0.850 | 0.425 | 0.56 | 2.02 |
| Ceramide(42:0) | (b) | 0.9 | 0.592 | 0.296 | 0.54 | 1.42 |
| Ceramide(42:1) | (b) | 1.2 | 0.523 | 0.262 | 0.68 | 2.14 |
| Ceramide(42:2) | (b) | 0.8 | 0.526 | 0.263 | 0.33 | 1.77 |
| Ceramide(43:1) | (b) | 0.9 | 0.827 | 0.414 | 0.46 | 1.86 |
| Cholesterol Ester(16:1) | (c) | 1.0 | 0.867 | 0.434 | 0.67 | 1.41 |
| Cholesterol Ester(20:4) | (c) | 2.6 | 0.004 | 0.002 | 1.36 | 4.83 |
| Cholesterol Ester(20:5) | (c) | 1.1 | 0.406 | 0.203 | 0.85 | 1.51 |
| Cholesterol Ester(22:6) | (c) | 1.1 | 0.805 | 0.403 | 0.72 | 1.54 |
| Diacylglycerol(32:0) | (d) | 0.9 | 0.298 | 0.149 | 0.74 | 1.10 |
| Diacylglycerol(34:0) | (d) | 0.9 | 0.258 | 0.129 | 0.67 | 1.12 |
| Diacylglycerol(34:1) | (d) | 0.9 | 0.384 | 0.192 | 0.66 | 1.18 |
| Diacylglycerol(34:2) | (d) | 0.9 | 0.524 | 0.262 | 0.67 | 1.22 |
| Diacylglycerol(36:3) | (d) | 0.9 | 0.420 | 0.210 | 0.58 | 1.26 |
| Diacylglycerol(36:4) | (d) | 1.0 | 0.981 | 0.490 | 0.74 | 1.36 |
| Diacylglycerol(37:7) | (d) | 0.9 | 0.333 | 0.166 | 0.76 | 1.10 |
| Leukotriene B4 | (e) | 0.3 | 0.166 | 0.083 | 0.07 | 1.57 |
| Prostaglandin D2; Prostaglandin E2 | (e) | 4.4 | 0.047 | 0.024 | 1.02 | 18.99 |
| Prostaglandin D2; Prostaglandin E2 | (e) | 1.5 | 0.463 | 0.232 | 0.52 | 4.23 |
| 4,7-dioxo-octanoic acid | (f) | 0.8 | 0.564 | 0.282 | 0.31 | 1.89 |
| Free Fatty Acid (18:1) | (f) | 1.1 | 0.429 | 0.214 | 0.92 | 1.23 |
| Free Fatty Acid (18:2) (linoleic acid) | (f) | 1.1 | 0.162 | 0.081 | 0.96 | 1.32 |
| Free Fatty Acid (20:4) (arachidonic acid) | (f) | 1.2 | 0.145 | 0.073 | 0.93 | 1.64 |
| Free Fatty Acid (22:6) | (f) | 1.0 | 0.953 | 0.477 | 0.80 | 1.27 |
| (docosahexaenoic acid) | | | | | | |
| Glucosyl/GalactosylCer(40:0) | (g) | 3.3 | 0.030 | 0.015 | 1.12 | 9.40 |
| GlucosylCeramide(42:1) | (g) | 1.3 | 0.501 | 0.251 | 0.63 | 2.57 |
| LactosylCeramide(18:1/16:0) | (g) | 1.6 | 0.247 | 0.123 | 0.72 | 3.53 |
| Lactosylceramide(18:1/20:4) | (g) | 1.0 | 0.909 | 0.455 | 0.46 | 2.01 |
| LactosylCeramide(32:0) | (g) | 2.0 | 0.016 | 0.008 | 1.14 | 3.51 |
| Lactosylceramide(32:0) | (g) | 1.4 | 0.388 | 0.194 | 0.68 | 2.75 |
| LactosylCeramide(34:1) | (g) | 1.8 | 0.199 | 0.100 | 0.75 | 4.09 |
| LactosylCeramide(36:0) | (g) | 1.9 | 0.039 | 0.019 | 1.03 | 3.52 |
| LactosylCeramide(36:0) | (g) | 2.9 | 0.055 | 0.027 | 0.98 | 8.36 |
| Trihexosylceramide(34:1) | (g) | 2.6 | 0.047 | 0.023 | 1.01 | 6.54 |
| Trihexosylceramide(40:1) | (g) | 2.2 | 0.069 | 0.034 | 0.94 | 5.16 |
| Lysophosphatidylcholine(14:0) | (h) | 0.9 | 0.727 | 0.363 | 0.55 | 1.52 |
| Lysophosphatidylcholine(15:0) | (h) | 1.1 | 0.887 | 0.444 | 0.46 | 2.44 |
| Lysophosphatidylcholine(15:1) | (h) | 1.0 | 0.979 | 0.489 | 0.56 | 1.77 |
| Lysophosphatidylcholine(16:0) | (h) | 1.0 | 0.948 | 0.474 | 0.45 | 2.34 |
| Lysophosphatidylcholine(16:1) | (h) | 1.0 | 0.919 | 0.460 | 0.59 | 1.81 |
| Lysophosphatidylcholine(17:0) | (h) | 1.3 | 0.373 | 0.187 | 0.71 | 2.46 |
| Lysophosphatidylcholine(17:1) | (h) | 1.3 | 0.390 | 0.195 | 0.70 | 2.54 |
| Lysophosphatidylcholine(17:2) | (h) | 1.4 | 0.219 | 0.109 | 0.81 | 2.53 |
| Lysophosphatidylcholine(18:0) | (h) | 1.3 | 0.475 | 0.237 | 0.63 | 2.69 |
| Lysophosphatidylcholine(18:1) | (h) | 1.5 | 0.201 | 0.100 | 0.82 | 2.60 |
| Lysophosphatidylcholine(18:2) | (h) | 1.4 | 0.225 | 0.113 | 0.81 | 2.45 |
| Lysophosphatidylcholine(18:3) | (h) | 1.4 | 0.125 | 0.062 | 0.92 | 2.05 |
| Lysophosphatidylcholine(20:0) | (h) | 1.2 | 0.258 | 0.129 | 0.86 | 1.75 |
| Lysophosphatidylcholine(20:1) | (h) | 1.2 | 0.535 | 0.268 | 0.68 | 2.11 |
| Lysophosphatidylcholine(20:2) | (h) | 1.4 | 0.420 | 0.210 | 0.63 | 3.09 |
| Lysophosphatidylcholine(20:3) | (h) | 1.1 | 0.728 | 0.364 | 0.69 | 1.69 |
| Lysophosphatidylcholine(20:4) | (h) | 1.1 | 0.402 | 0.201 | 0.84 | 1.57 |
| Lysophosphatidylcholine(20:5) | (h) | 1.1 | 0.597 | 0.299 | 0.85 | 1.34 |
| Lysophosphatidylcholine(22:4) | (h) | 1.2 | 0.456 | 0.228 | 0.76 | 1.85 |
| Lysophosphatidylcholine(22:5) | (h) | 1.1 | 0.489 | 0.244 | 0.80 | 1.59 |
| Lysophosphatidylcholine(22:6) | (h) | 1.2 | 0.357 | 0.179 | 0.83 | 1.66 |
| Lysophosphatidylcholine(24:0) | (h) | 1.4 | 0.365 | 0.183 | 0.67 | 3.00 |
| Lysophosphatidylcholine(26:0) | (h) | 1.8 | 0.280 | 0.140 | 0.62 | 5.29 |
| Lysophosphatidylethanolamine(16:0) | (h) | 1.2 | 0.543 | 0.271 | 0.62 | 2.49 |
| Lysophosphatidylethanolamine(18:0) | (h) | 1.4 | 0.395 | 0.197 | 0.67 | 2.76 |
| Lysophosphatidylethanolamine(18:1) | (h) | 1.4 | 0.102 | 0.051 | 0.94 | 2.01 |
| Lysophosphatidylethanolamine(18:2) | (h) | 1.3 | 0.330 | 0.165 | 0.78 | 2.11 |
| Lysophosphatidylethanolamine(20:3) | (h) | 1.9 | 0.124 | 0.062 | 0.84 | 4.27 |
| Lysophosphatidylethanolamine(20:4) | (h) | 1.3 | 0.374 | 0.187 | 0.74 | 2.22 |
| Lysophosphatidylethanolamine(22:0) | (h) | 1.6 | 0.336 | 0.168 | 0.62 | 4.14 |
| Lysophosphatidylethanolamine(22:6) | (h) | 1.1 | 0.634 | 0.317 | 0.68 | 1.88 |
| Plas_Lysophosphatidylcholine(P-18:0/0:0) | (h) | 1.1 | 0.822 | 0.411 | 0.57 | 2.04 |
| or Plas_Lysophosphatidylcholine(O-18:1) | | | | | | |
| PlasLysophosphatidylcholine(P-16:0) | (h) | 1.7 | 0.087 | 0.043 | 0.93 | 3.07 |
| PlasLysophosphatidylethanolamine(p-22:0) | (h) | 1.5 | 0.268 | 0.134 | 0.75 | 2.84 |
| 1-Linoleoylglycerol | (i) | 0.5 | 0.641 | 0.320 | 0.03 | 8.13 |
| 2-Arachidonylglycerol | (i) | 1.3 | 0.112 | 0.056 | 0.95 | 1.69 |
| Monoacylglycerol(20:0/0:0/0:0) | (i) | 1.2 | 0.676 | 0.338 | 0.51 | 2.83 |
| Phosphatidylcholine(30:1) | (j) | 1.1 | 0.666 | 0.333 | 0.75 | 1.58 |
| Phosphatidylcholine(32:0) | (j) | 1.2 | 0.559 | 0.279 | 0.60 | 2.58 |
| Phosphatidylcholine(32:1) | (j) | 0.9 | 0.450 | 0.225 | 0.60 | 1.25 |
| Phosphatidylcholine(32:2) | (j) | 0.8 | 0.494 | 0.247 | 0.52 | 1.37 |
| Phosphatidylcholine(33:5) | (j) | 0.9 | 0.815 | 0.407 | 0.53 | 1.66 |
| Phosphatidylcholine(34:1) | (j) | 0.9 | 0.799 | 0.399 | 0.33 | 2.33 |
| Phosphatidylcholine(34:4) | (j) | 0.9 | 0.420 | 0.210 | 0.59 | 1.25 |
| Phosphatidylcholine(35:1); | (j) | 1.2 | 0.650 | 0.325 | 0.56 | 2.53 |
| Phosphatidylethanolamine(38:1) | | | | | | |
| Phosphatidylcholine(35:2) | (j) | 1.0 | 0.965 | 0.482 | 0.47 | 2.19 |
| Phosphatidylcholine(36:1) | (j) | 1.3 | 0.492 | 0.246 | 0.63 | 2.64 |
| Phosphatidylcholine(36:2) | (j) | 1.2 | 0.818 | 0.409 | 0.34 | 3.93 |
| Phosphatidylcholine(36:4) | (j) | 1.6 | 0.279 | 0.140 | 0.68 | 3.79 |
| Phosphatidylcholine(36:5) | (j) | 1.1 | 0.333 | 0.166 | 0.88 | 1.45 |
| Phosphatidylcholine(38:5) | (j) | 2.4 | 0.022 | 0.011 | 1.13 | 4.96 |
| Phosphatidylcholine(38:6) | (j) | 1.0 | 0.906 | 0.453 | 0.55 | 1.97 |
| Phosphatidylcholine(38:7) | (j) | 1.1 | 0.769 | 0.384 | 0.62 | 1.90 |
| Phosphatidylcholine(40:5) | (j) | 1.3 | 0.237 | 0.118 | 0.85 | 1.98 |
| Phosphatidylcholine(40:7) | (j) | 1.3 | 0.397 | 0.199 | 0.74 | 2.12 |
| Phosphatidylcholine(40:8) | (j) | 1.4 | 0.352 | 0.176 | 0.70 | 2.73 |
| Phosphatidylethanolamine(36:1) | (j) | 1.2 | 0.476 | 0.238 | 0.72 | 2.02 |
| Phosphatidylethanolamine(36:1) | (j) | 1.0 | 0.998 | 0.499 | 0.61 | 1.65 |
| Phosphatidylethanolamine(38:4) | (j) | 1.1 | 0.589 | 0.295 | 0.73 | 1.75 |
| Phosphatidylglycerol(43:0) | (j) | 1.6 | 0.294 | 0.147 | 0.68 | 3.64 |
| Phosphatidylserine(41:1) | (j) | 1.3 | 0.562 | 0.281 | 0.57 | 2.84 |
| Plas_Phosphatidylcholine(o-30:1) or | (j) | 0.8 | 0.615 | 0.307 | 0.28 | 2.13 |
| Plas_Phosphatidylcholine(p-30:0) | | | | | | |
| Plas_Phosphatidylcholine(o-32:1) or | (j) | 1.2 | 0.628 | 0.314 | 0.57 | 2.56 |
| Plas_Phosphatidylcholine(p-32:0) | | | | | | |
| Plas_Phosphatidylcholine(o-34:1) or | (j) | 1.2 | 0.620 | 0.310 | 0.53 | 2.92 |
| Plas_Phosphatidylcholine(p-34:0) | | | | | | |
| Plas_Phosphatidylcholine(o-34:3) or | (j) | 1.1 | 0.220 | 0.110 | 0.94 | 1.32 |
| Plas_Phosphatidylcholine(p-34:2) | | | | | | |
| Plas_Phosphatidylcholine(o-38:6) or | (j) | 1.9 | 0.084 | 0.042 | 0.92 | 4.03 |
| Plas_Phosphatidylcholine(p-38:5) | | | | | | |
| Plas_Phosphatidylcholine(o-38:7) or | (j) | 1.7 | 0.066 | 0.033 | 0.97 | 3.01 |
| Plas_Phosphatidylcholine(p-38:6) | | | | | | |
| Plas_Phosphatidylcholine(o-40:6) or | (j) | 1.5 | 0.230 | 0.115 | 0.78 | 2.88 |
| Plas_Phosphatidylcholine(p-40:5) | | | | | | |
| Plas_Phosphatidylcholine(o-40:7) or | (j) | 1.9 | 0.026 | 0.013 | 1.08 | 3.29 |
| Plas_Phosphatidylcholine(p-40:6) | | | | | | |
| Plas_Phosphatidylethanolamine(o-38:5) or | (j) | 1.4 | 0.198 | 0.099 | 0.85 | 2.24 |
| Plas_Phosphatidylethanolamine(p-38:4) | | | | | | |
| Plas_Phosphatidylethanolamine(o-40:5) or | (j) | 1.9 | 0.023 | 0.011 | 1.09 | 3.37 |
| Plas_Phosphatidylethanolamine(p-40:4) | | | | | | |
| Plas_Phosphatidylethanolamine(o-40:5) or | (j) | 1.2 | 0.432 | 0.216 | 0.74 | 2.03 |
| Plas_Phosphatidylethanolamine(p-40:4) | | | | | | |
| Plas_Phosphatidylethanolamine(o-40:6) or | (j) | 1.5 | 0.179 | 0.089 | 0.84 | 2.49 |
| Plas_Phosphatidylethanolamine(p-40:5) | | | | | | |
| Plas_Phosphatidylserine(p-39:1) | (j) | 1.2 | 0.680 | 0.340 | 0.45 | 3.38 |
| Plas_Phosphatidylserine(p-40:1) | (j) | 1.0 | 0.916 | 0.458 | 0.53 | 2.02 |
| PlasPhosphatidylcholine(o-32:1) or | (j) | 2.1 | 0.059 | 0.029 | 0.97 | 4.51 |
| PlasPhosphatidylcholine(p-32:0) | | | | | | |
| PlasPhosphatidylcholine(o-34:1) or | (j) | 1.4 | 0.420 | 0.210 | 0.61 | 3.25 |
| PlasPhosphatidylcholine(p-34:0) | | | | | | |
| PlasPhosphatidylcholine(o-40:2) or | (j) | 2.4 | 0.036 | 0.018 | 1.06 | 5.56 |
| PlasPhosphatidylcholine(p-40:1) | | | | | | |
| PlasPhosphatidylcholine(o-40:7) or | (j) | 1.4 | 0.348 | 0.174 | 0.72 | 2.59 |
| PlasPhosphatidylcholine(p-40:6) | | | | | | |
| PlasPhosphatidylcholine(o-42:5) or | (j) | 1.3 | 0.554 | 0.277 | 0.60 | 2.58 |
| PlasPhosphatidylcholine(p-42:4) | | | | | | |
| PlasPhosphatidylethanolamine(o-38:5) or | (j) | 1.5 | 0.152 | 0.076 | 0.87 | 2.44 |
| PlasPhosphatidylethanolamine(p-38:4) | | | | | | |
| Sphingomyelin(32:1) | (k) | 1.2 | 0.534 | 0.267 | 0.70 | 2.01 |
| Sphingomyelin(32:2) | (k) | 1.2 | 0.503 | 0.252 | 0.69 | 2.15 |
| Sphingomyelin(33:1) | (k) | 2.4 | 0.091 | 0.046 | 0.87 | 6.34 |
| Sphingomyelin(33:2) | (k) | 1.4 | 0.447 | 0.224 | 0.57 | 3.59 |
| Sphingomyelin(34:0) | (k) | 1.7 | 0.167 | 0.084 | 0.79 | 3.78 |
| Sphingomyelin(34:1) | (k) | 2.8 | 0.091 | 0.045 | 0.85 | 8.90 |
| Sphingomyelin(34:2) | (k) | 1.5 | 0.337 | 0.168 | 0.67 | 3.29 |
| Sphingomyelin(36:1) | (k) | 1.5 | 0.277 | 0.138 | 0.73 | 2.95 |
| Sphingomyelin(36:2) | (k) | 1.5 | 0.233 | 0.117 | 0.76 | 3.11 |
| Sphingomyelin(36:3) | (k) | 1.2 | 0.441 | 0.221 | 0.72 | 2.14 |
| Sphingomyelin(38:1) | (k) | 1.5 | 0.274 | 0.137 | 0.74 | 2.89 |
| Sphingomyelin(39:2) | (k) | 1.9 | 0.161 | 0.081 | 0.77 | 4.71 |
| Sphingomyelin(40:1) | (k) | 1.7 | 0.119 | 0.060 | 0.88 | 3.09 |
| Sphingomyelin(40:2) | (k) | 2.2 | 0.070 | 0.035 | 0.94 | 5.19 |
| Sphingomyelin(40:3) | (k) | 1.4 | 0.285 | 0.143 | 0.76 | 2.52 |
| Sphingomyelin(41:1) | (k) | 2.0 | 0.087 | 0.044 | 0.90 | 4.42 |
| Sphingomyelin(42:1) | (k) | 1.8 | 0.089 | 0.045 | 0.91 | 3.51 |
| Sphingomyelin(42:2) | (k) | 1.7 | 0.140 | 0.070 | 0.83 | 3.61 |
| Sphingomyelin(42:3) | (k) | 1.6 | 0.242 | 0.121 | 0.72 | 3.62 |
| Sphingomyelin(43:2) | (k) | 1.4 | 0.350 | 0.175 | 0.70 | 2.71 |
| Sphingomyelin(44:2) | (k) | 3.9 | 0.007 | 0.004 | 1.45 | 10.60 |
| Triacylglycerol(16:0_18:1_18:2) | (l) | 0.4 | 0.459 | 0.229 | 0.05 | 4.04 |
| Triacylglycerol(40:0) | (l) | 1.0 | 0.115 | 0.057 | 0.99 | 1.06 |
| Triacylglycerol(42:2) | (l) | 1.1 | 0.123 | 0.062 | 0.99 | 1.11 |
| Triacylglycerol(44:0) | (l) | 1.0 | 0.450 | 0.225 | 0.94 | 1.16 |
| Triacylglycerol(44:1) | (l) | 1.0 | 0.795 | 0.397 | 0.87 | 1.12 |
| Triacylglycerol(44:2) | (l) | 1.0 | 0.967 | 0.483 | 0.87 | 1.16 |
| Triacylglycerol(45:0) | (l) | 1.0 | 0.992 | 0.496 | 0.71 | 1.41 |
| Triacylglycerol(46:0) | (l) | 1.3 | 0.107 | 0.053 | 0.94 | 1.87 |
| Triacylglycerol(46:1) | (l) | 1.0 | 0.636 | 0.318 | 0.85 | 1.10 |
| Triacylglycerol(46:2) | (l) | 0.9 | 0.503 | 0.252 | 0.74 | 1.16 |
| Triacylglycerol(46:3) | (l) | 1.0 | 0.935 | 0.468 | 0.80 | 1.23 |
| Triacylglycerol(47:0) | (l) | 1.2 | 0.567 | 0.284 | 0.59 | 2.63 |
| Triacylglycerol(47:3) | (l) | 1.0 | 0.981 | 0.490 | 0.72 | 1.40 |
| Triacylglycerol(48:0) | (l) | 1.1 | 0.787 | 0.393 | 0.67 | 1.71 |
| Triacylglycerol(48:1) | (l) | 1.0 | 0.949 | 0.474 | 0.70 | 1.41 |
| Triacylglycerol(48:2) | (l) | 1.0 | 0.934 | 0.467 | 0.63 | 1.53 |
| Triacylglycerol(48:3) | (l) | 0.9 | 0.660 | 0.330 | 0.66 | 1.30 |
| Triacylglycerol(48:3) | (l) | 1.0 | 0.849 | 0.425 | 0.83 | 1.26 |
| Triacylglycerol(48:4) | (l) | 1.0 | 0.792 | 0.396 | 0.82 | 1.31 |
| Triacylglycerol(49:1) | (l) | 0.9 | 0.601 | 0.300 | 0.71 | 1.22 |
| Triacylglycerol(49:2) | (l) | 0.8 | 0.275 | 0.138 | 0.62 | 1.15 |
| Triacylglycerol(49:3) | (l) | 0.9 | 0.614 | 0.307 | 0.57 | 1.39 |
| Triacylglycerol(50:1) | (l) | 1.1 | 0.706 | 0.353 | 0.59 | 2.19 |
| Triacylglycerol(50:3) | (l) | 0.8 | 0.461 | 0.231 | 0.38 | 1.56 |
| Triacylglycerol(50:5) | (l) | 1.1 | 0.617 | 0.308 | 0.74 | 1.67 |
| Triacylglycerol(51:1) | (l) | 1.1 | 0.570 | 0.285 | 0.78 | 1.57 |
| Triacylglycerol(51:2) | (l) | 0.9 | 0.648 | 0.324 | 0.54 | 1.48 |
| Triacylglycerol(51:3) | (l) | 0.8 | 0.360 | 0.180 | 0.43 | 1.36 |
| Triacylglycerol(51:4) | (l) | 0.9 | 0.685 | 0.342 | 0.63 | 1.36 |
| Triacylglycerol(52:2) | (l) | 1.0 | 0.943 | 0.471 | 0.22 | 4.06 |
| Triacylglycerol(52:4) | (l) | 0.7 | 0.575 | 0.288 | 0.16 | 2.79 |
| Triacylglycerol(52:7) | (l) | 1.5 | 0.092 | 0.046 | 0.94 | 2.34 |
| Triacylglycerol(53:1) | (l) | 0.9 | 0.624 | 0.312 | 0.66 | 1.29 |
| Triacylglycerol(53:2) | (l) | 1.1 | 0.657 | 0.328 | 0.72 | 1.70 |
| Triacylglycerol(53:3) | (l) | 0.8 | 0.580 | 0.290 | 0.44 | 1.59 |
| Triacylglycerol(53:4) | (l) | 0.9 | 0.755 | 0.378 | 0.56 | 1.53 |
| Triacylglycerol(54:3) | (l) | 1.0 | 0.879 | 0.440 | 0.60 | 1.56 |
| Triacylglycerol(54:4) | (l) | 1.7 | 0.320 | 0.160 | 0.61 | 4.54 |
| Triacylglycerol(54:6) | (l) | 1.3 | 0.322 | 0.161 | 0.77 | 2.18 |
| Triacylglycerol(55:1) | (l) | 1.2 | 0.602 | 0.301 | 0.66 | 2.06 |
| Triacylglycerol(55:2) | (l) | 0.5 | 0.246 | 0.123 | 0.19 | 1.52 |
| Triacylglycerol(55:2) | (l) | 1.0 | 0.887 | 0.444 | 0.52 | 1.77 |
| Triacylglycerol(56:7) | (l) | 1.1 | 0.732 | 0.366 | 0.80 | 1.37 |
| Triacylglycerol(56:9) | (l) | 1.4 | 0.032 | 0.016 | 1.03 | 1.97 |
| Triacylglycerol(57:3) | (l) | 1.2 | 0.736 | 0.368 | 0.36 | 4.33 |
| Triacylglycerol(57:8) | (l) | 1.1 | 0.652 | 0.326 | 0.75 | 1.58 |
| Triacylglycerol(58:9) | (l) | 1.3 | 0.063 | 0.031 | 0.99 | 1.68 |
| Triacylglycerol(59:4) | (l) | 1.3 | 0.419 | 0.210 | 0.67 | 2.60 |
| Triacylglycerol(59:5) | (l) | 1.3 | 0.330 | 0.165 | 0.77 | 2.19 |
| Triacylglycerol(60:3) | (l) | 1.1 | 0.892 | 0.446 | 0.46 | 2.46 |
| Triacylglycerol(61:3) | (l) | 1.0 | 0.955 | 0.477 | 0.45 | 2.32 |
| Triacylglycerol(61:6) | (l) | 1.4 | 0.347 | 0.174 | 0.68 | 2.98 |
| Triacylglycerol(61:7) | (l) | 1.4 | 0.239 | 0.119 | 0.79 | 2.59 |
| Triacylglycerol(65:11) | (l) | 1.2 | 0.177 | 0.088 | 0.91 | 1.64 |

# treated as continuous variables
^{‡}p-values
(a) acylcarnitine
(b) ceramide
(c) cholesterol ester
(d) diacylglycerol
(e) eicosanoid
(f) free fatty acid
(g) glycosphingolipid
(h) lysophospholipid
(i) monoacylglycerol
(j) phospholipid
(k) sphingolipid
(l) triacylglycerol

Next, from the set of sphingolipids that exhibited statistically significant (p<0.05) HRs and, using a logistic regression model, a signature panel was developed which comprised plasma Cav-1 and six sphingolipids that exhibited positive β-estimates in the logistic regression model: SM(40:2), SM(44:2), lactosylceramide(32:0), lactosylceramide(36:0), trihexosylceramide(34:1) and hexosylceramide(40:0). Using log rank test statistics from the Cox model, an optimal cut-off point was calculated for the plasma Cav-1-sphingolipid signature that would yield the greatest difference between subjects on AS that exhibited disease progression (defined as upgrading of GS and/or increased tumor volume) from those that did not. This resulted in a cut-off value of 4.33. Assessment of the association of the signature with progression free survival was achieved using Cox-proportional hazard models. In a multivariate analyses, adjusted for age, 5-alpha reductase treatment, and baseline tumor volume, AS subjects with a plasma Cav-1-sphingolipid signature score>4.33 exhibited statistically significantly worse DP free survival as compared to those with a plasma Cav-1-sphingolipid signature score≤4.33 (HR: 2.70, 95% CI: 1.75-4.16, p-value: <0.001) (Table 5). Notably, non-proportionality hazard model tests yielded non-significant p-values. Of relevance, in this analyses BMI was not associated with increased risk of DP (HR: 1.02, 95% CI: 0.40-2.64, p-value: 0.965) indicating that this lipid signature is unlikely to be biased by obesity.

TABLE 5
Hazard models for the Cav-1-sphingolipid signature
and disease progression free-survival

| | Univariable | | | Multivariable† | | |
|---|---|---|---|---|---|---|
| Variable | HR | 95% CI | 2-sided P | HR | 95% CI | 2-sided P |
| Age | | | | | | |
| <64 | Reference | | | Reference | | |
| ≥64 | 1.66 | 1.11-2.48 | 0.014 | 1.68 | 1.12-2.53 | 0.013 |
| BMI\hat{ } | 1.02 | 0.40-2.64 | 0.965 | — | | |
| PSA Density‡ | 3.77 | 0.99-14.44 | 0.053 | — | | |
| 5-ARI treatment | | | | | | |
| No | Reference | | | Reference | | |
| Yes | 0.50 | 0.23-1.07 | 0.074 | 0.37 | 0.17-0.82 | 0.014 |
| Risk Group | | | | | | |
| I | Reference | | | Reference | | |
| II | 2.94 | 1.75-4.96 | <0.001 | 2.60 | 1.54-4.38 | <0.001 |
| Sphingolipid Signature | | | | | | |
| Below Cutoff (≤4.33) | Reference | | | Reference | | |
| Above Cutoff (>4.33) | 2.62 | 1.71-4.01 | <0.001 | 2.70 | 1.75-4.16 | <0.001 |

Kaplan-Meier survival curves depicting progression free survival for participants with plasma Cav-1—sphingolipid signature scores below (<4.33) or equal to or above the cutoff (>4.33) are provided in Table 6 and FIG. 1C.

TABLE 6
Progression-free survival

| signature | time(months) | | | | | | | | | | | | |
|---|---|---|---|---|---|---|---|---|---|---|---|---|---|
| cutoff | 0 | 10 | 20 | 30 | 40 | 50 | 60 | 70 | 80 | 90 | 100 | 110 | 120 |
| ≤4.33 | 383 | 378 | 269 | 229 | 165 | 109 | 92 | 49 | 27 | 18 | 8 | 2 | 1 |
| >4.33 | 76 | 75 | 52 | 41 | 27 | 16 | 13 | 3 | 1 | 1 | 1 | 0 | 0 |

Multivariate Cox-proportional hazard models for the Cav-1-sphingolipid signature and its association with disease progression free-survival. Cox proportional hazard models using a plasma Cav-1-sphingolipid signature cut-off value of 4.33. Optimal cut-off values for plasma Cav-1-sphingolipid signature were derived using log rank statistic based methods as previously described. Age, 5-α reductase treatment and baseline tumor volume (Risk Group 1: 1 positive biopsy core with tumor focus of <3.0 mm in Gleason 3 + 3 = 6 patients or <2.0 mm in Gleason 3 + 4 = 7 patients; Risk Group 2: >1 core or baseline tumor length greater than those of Risk Group 1) were included as co-variables based on a backward stepwise selection method (likelihood ratio).
† Variables included into the equation after selection using a backward stepwise method (likelihood ratio)
\hat{ } per unit log 2 increase
‡ per unit increase

To better define the relationship between lipid metabolism and Cav-1 within the context of the Cav-1—sphingolipid signature, gene expression profiles reflective of lipid managing apparati and mRNA expression of CAV1 in 333 prostate tumors using The Cancer Genome Atlas (TCGA) were first compared. High CAV1 mRNA expression was found to be positively associated with genes annotated to ontologies related to lipid scavenging and metabolism, glycosylceramide metabolic process as well as the ceramide pathway. Association of elevated CAV1 and associated lipid managing apparti with previously defined molecular subtypes (iClusters) of primary prostate cancers was next probed. The results indicated that high CAV1 mRNA expression was predominately associated with iCluster 3, which is characterized by elevated PI3K/AKT, MAP-Kinase and receptor tyrosine kinase activity.

### Example 6: Association of Cav-1 with a High-Lipid Scavenging Phenotype

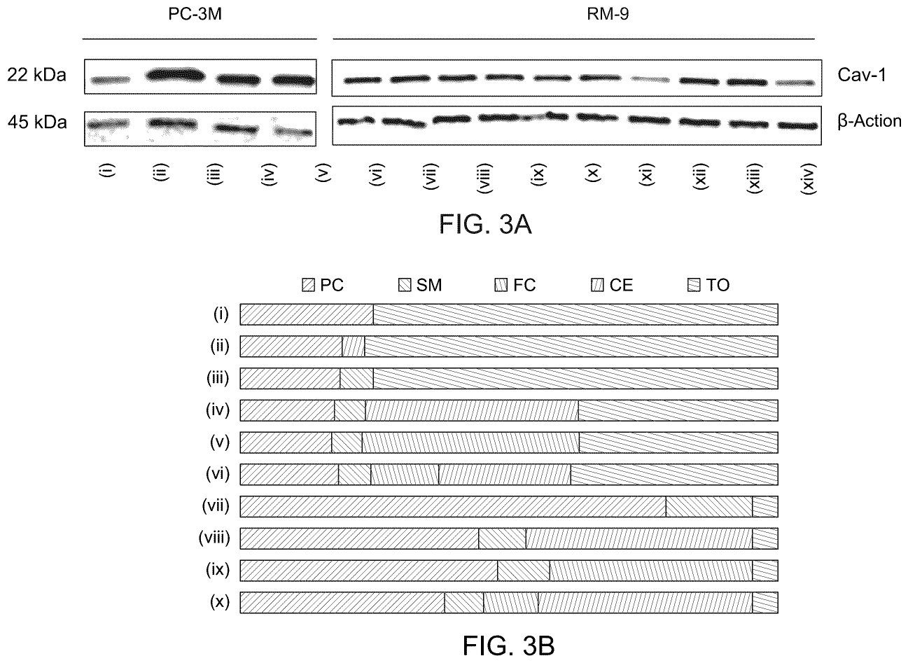

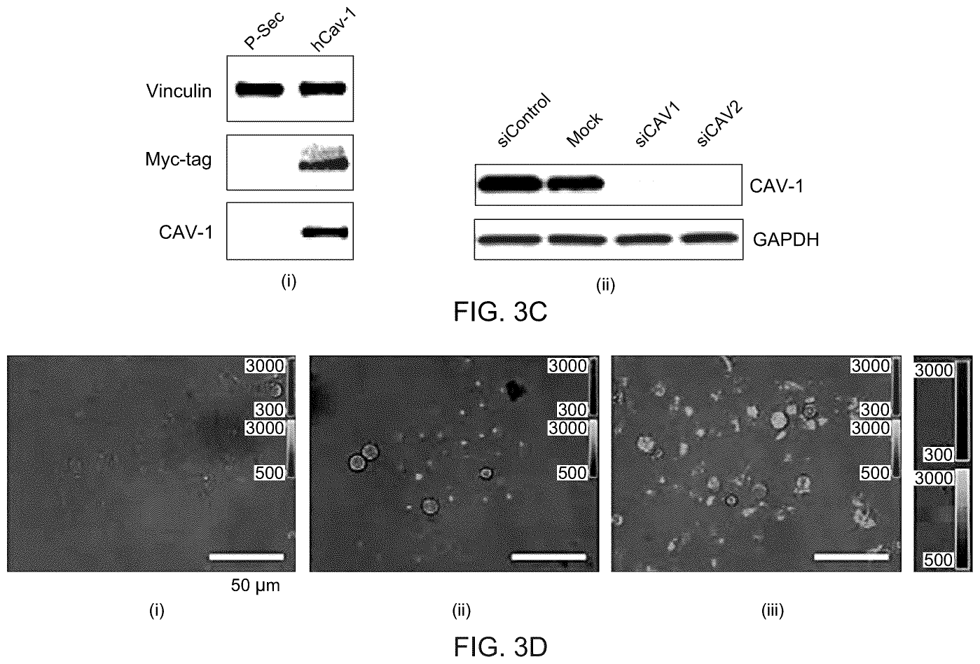

The response of Cav-1 to extracellular lipid availability was next assessed. In comparing serum-free media with and without lipids, lipid-deprivation reduced Cav-1 protein expression in RM-9 and PC-3M prostate cancer cell lines (FIGS. 3A and 3B). Notably, the presence of apolipoproteins, cholesterol or cholesterol esters was dispensable for maintaining elevated Cav-1 protein levels, suggesting that Cav-1 is specifically responsive to the phospholipid constituents of extracellular lipid complexes (FIGS. 3A and 3B).

Participation of Cav-1 in lipid uptake was next explored. To this end, the ability of Cav-1 low FIG. 3B) LNCaP and Cav-1 positive (FIG. 3A) PC-3M and RM-9 prostate cancer cell lines to scavenge extracellular fluorescent DiI-conjugated SSALPs was next assessed. PC-3M and RM-9 prostate cancer cells exhibited substantially higher fluorescence accumulation as compared to LNCaP (FIG. 3D).

### Example 7: Regulation of Glycosphingolipid Biosynthesis by Cav-1

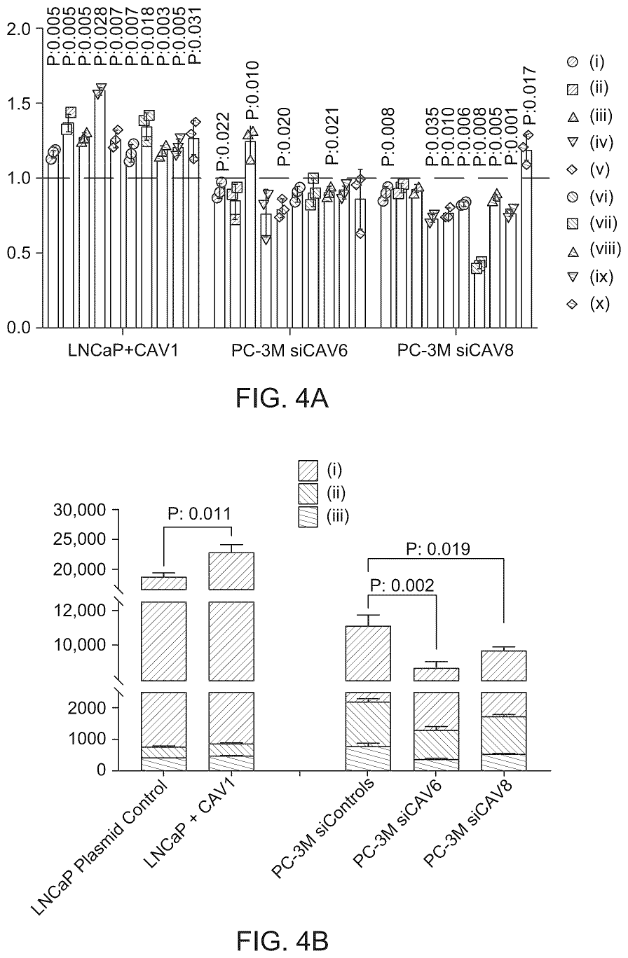

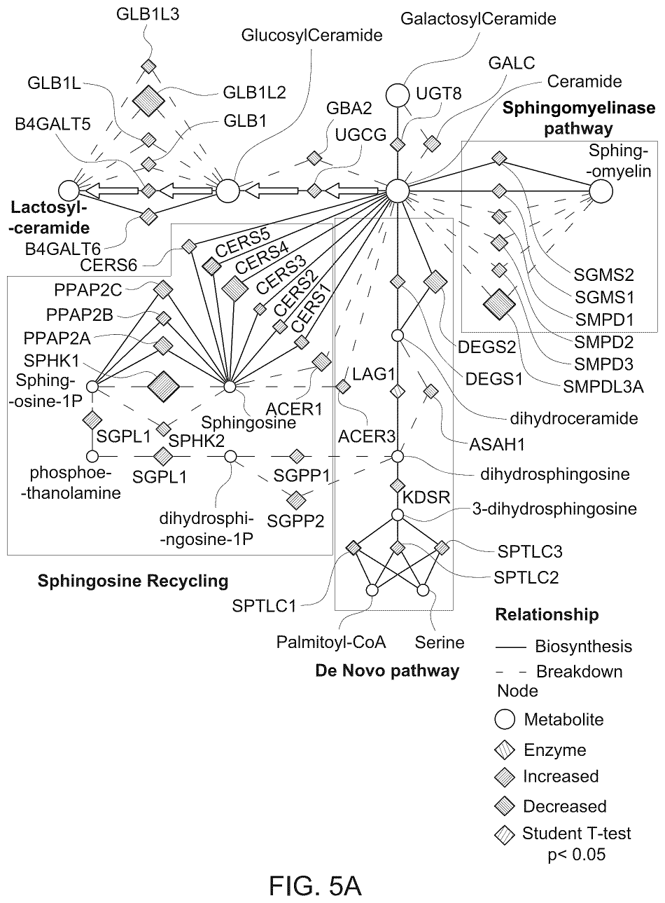

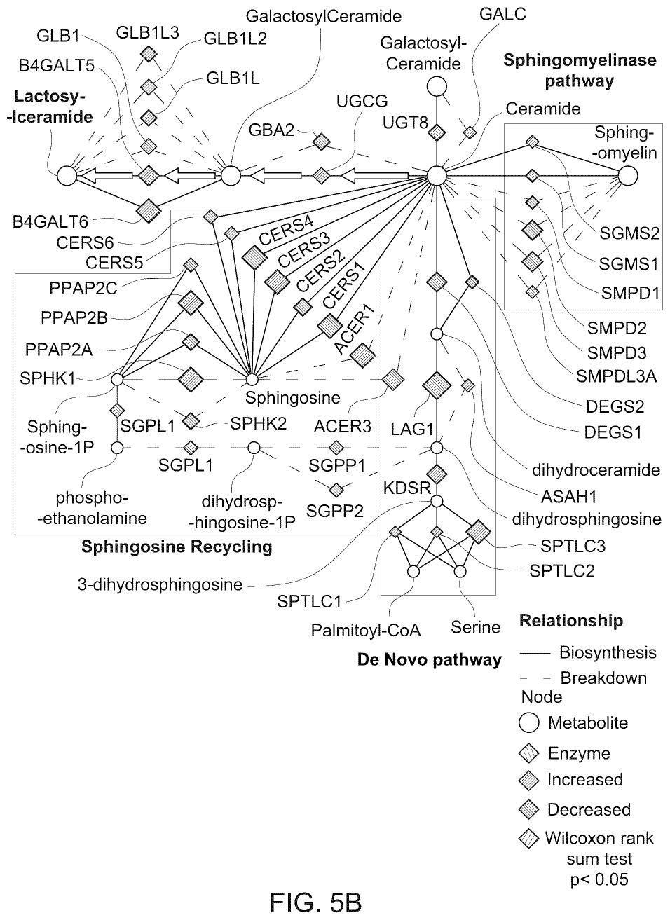

Comparison of baseline lipid profiles of LNCaP and PC-3M cells was next performed, as well as lipid profiles following respective overexpression of Cav-1, or transient knockdown of CAV1. Immunoblots comparing whole cell lysate Cav-1 levels in LNCaP and PC-3M cells following respective overexpression of Cav-1 or transient knockdown of CAV1 are provided in FIG. 3B. Relative to baseline LNCaP, baseline PC-3M cells exhibited elevated levels of triacylglycerols, cholesterol esters and lysophospholipids, whereas sphingolipids were reduced. Overexpression of Cav-1 resulted in significant increases in overall levels of major lipid classes whereas Cav-1 knockdown resulted in significant reductions in phospholipids, diacylglycerols, sphingomyelins and glycosphingolipids, particularly lactosylceramides (FIGS. 4A and 4B). The CCLE and TCGA gene expression datasets were then utilized to evaluate the relationship between Cav-1 and enzymes central to ceramide metabolism. For CCLE data, prostate cancer cell lines were stratified based on mean CAV1 gene expression into either high CAV1 expressing cell lines (log 2 values>11 (range 11-01-13.61); HPrEC, DU145, PC-3) or low CAV1 expressing cell lines (log 2 values<7 (range 4.16-6.88); NCIH660, MDAPCa2B, LNCaP, VCaP, CWR22Rv1). TCGA data on prostate adenocarcinomas were stratified into the highest or lowest CAV1 expression quartiles to evaluate the association between CAV1 mRNA expression and mRNA expression of genes involved in sphingolipid metabolism amongst the most differential populations. Spearman correlation analyses based on the entire TCGA prostate adenocarcinoma dataset using continuous values for mRNA expression of CAV1 and genes involved in sphingolipid metabolism are provided in Table 7. As compared to those with low CAV1 mRNA levels, prostate cancer cell lines and prostate tumors exhibiting high CAV1 mRNA levels tended to also exhibit reduced mRNA expression levels for genes involved in the biogenesis of ceramides including dihydroceramide desaturase enzymes (DEGS), ceramide synthase enzymes (CERS) and sphingomyelinases (SPMDs) (FIGS. 5A and 5B). In contrast, mRNA expression of enzymes involved in the biosynthesis of glycosphingolipids, including glucosylceramide synthase (UCGC), and lactosylceramide synthases B4GALT5 and B4GALT6, were elevated in CAV1-high prostate cancer cell lines and prostate tumors.

TABLE 7
Spearman correlation analysis

| Pathway | Gene | Spearman Rho | 2-sided P |
|---|---|---|---|
| (a) | ASAH1 | 0.07 | 0.1629 |
| (a) | DEGS1 | −0.2 | 0.0001 |
| (a) | DEGS2 | 0.07 | 0.1684 |
| (a) | KDSR | 0.4 | <0.0001 |
| (a) | SPTLC1 | 0.03 | 0.5933 |
| (a) | SPTLC2 | 0.03 | 0.5189 |
| (a) | SPTLC3 | 0.37 | <0.0001 |
| (b) | ACER1 | 0.38 | <0.0001 |
| (b) | ACER2 | 0 | 0.9855 |
| (b) | ACER3 | −0.12 | 0.0186 |
| (b) | CERS1 | −0.31 | <0.0001 |
| (b) | CERS2 | −0.32 | <0.0001 |
| (b) | CERS3 | 0.38 | <0.0001 |
| (b) | CERS4 | −0.41 | <0.0001 |
| (b) | CERS5 | 0.09 | 0.1009 |
| (b) | CERS6 | 0.01 | 0.8473 |
| (b) | PLPP1 (PPAP2A) | −0.2 | 0.0001 |
| (b) | PLPP2 (PPAP2C) | −0.07 | 0.1786 |
| (b) | PLPP3 (PPAP2B) | 0.73 | <0.0001 |
| (b) | SGPL1 | −0.11 | 0.0336 |
| (b) | SGPP1 | −0.11 | 0.0353 |
| (b) | SGPP2 | 0.03 | 0.5302 |
| (b) | SPHK1 | 0.14 | 0.0076 |
| (b) | SPHK2 | −0.27 | <0.0001 |
| (c) | SGMS1 | −0.19 | 0.0004 |
| (c) | SGMS2 | 0.09 | 0.0941 |
| (c) | SMPD1 | −0.16 | 0.0028 |
| (c) | SMPD2 | −0.29 | <0.0001 |
| (c) | SMPD3 | −0.11 | 0.0336 |
| (c) | SMPDL3A | 0.02 | 0.7576 |
| (c) | SMPDL3A | 0.02 | 0.7576 |
| (d) | GBA2 | −0.29 | <0.0001 |
| (d) | GLB1 | −0.14 | 0.0074 |
| (d) | GALC | 0.01 | 0.8319 |
| (d) | GLB1L2 | 0.09 | 0.0961 |
| (d) | GLB1L | 0.1 | 0.0576 |
| (d) | UGCG | 0.1 | 0.0566 |
| (d) | GLB1L3 | 0.18 | 0.0008 |
| (d) | UGT8 | 0.28 | <0.0001 |
| (d) | B4GALT5 | 0.45 | <0.0001 |
| (d) | B4GALT6 | 0.45 | <0.0001 |

(a) de novo pathway
(b) sphingosine recycling
(c) sphingomyelinase pathway
(d) glycosphingolipid metabolism

### Example 8: Sphingomyelins as a Source of Ceramides and Glycosphingolipids

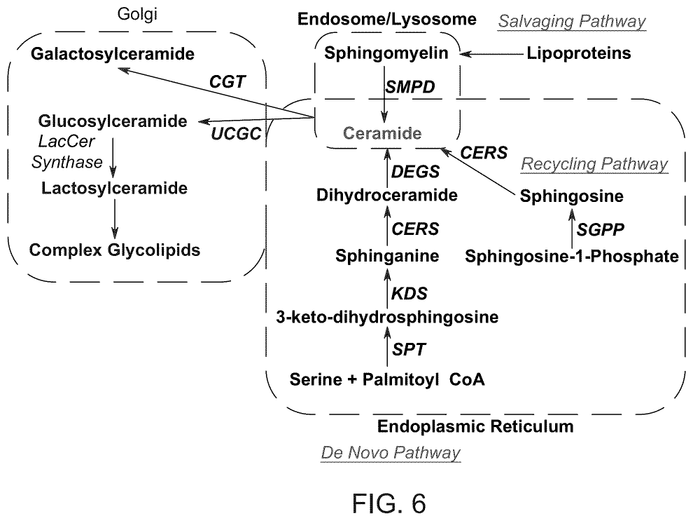

Investigation of the uptake of extracellular sphingomyelins as a potential source of ceramides and (via glycosylation), their glycosphingolipid derivatives. Ceramides are principally derived through three metabolic pathways: de novo, recycling or salvaging (FIG. 6). Ceramide biosynthesis through the salvaging pathway is mediated via the hydrolysis of sphingomyelins via sphingomyelinases.

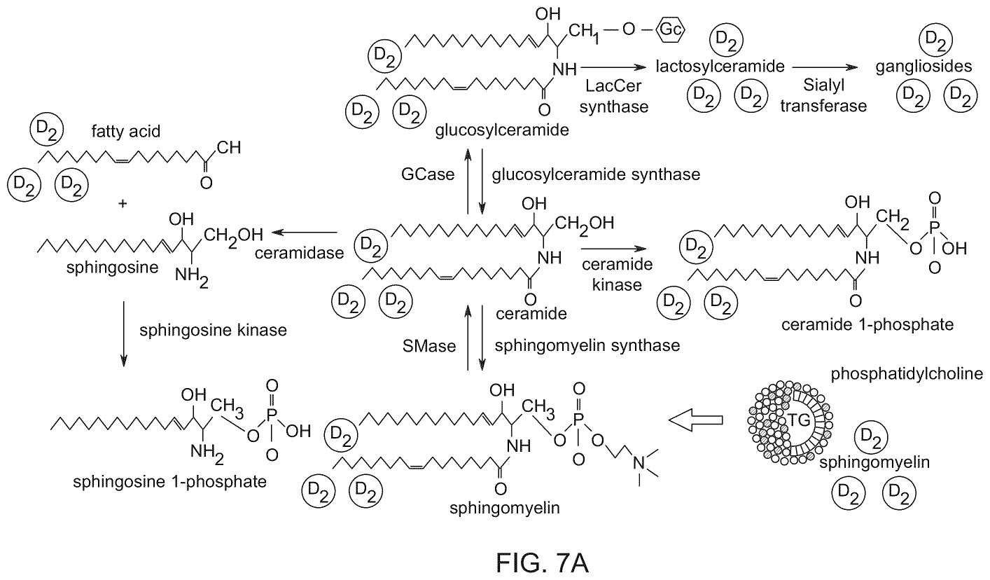

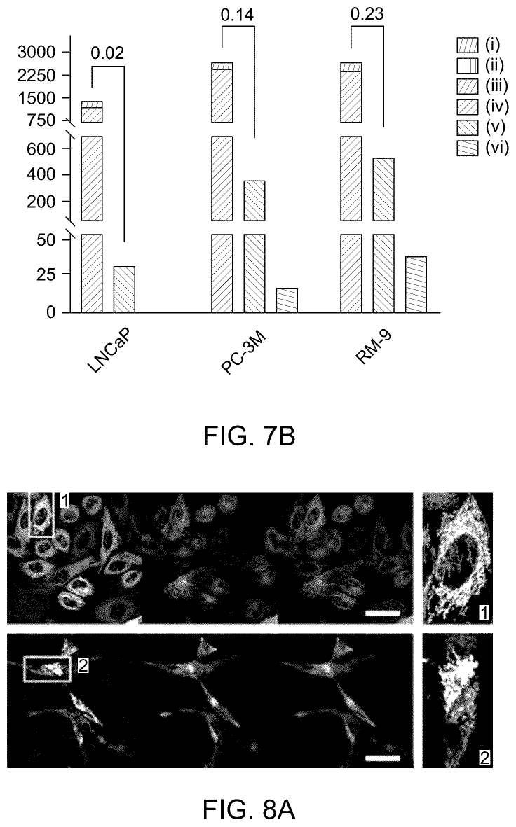

PC-3M, RM-9 and LNCaP prostate cancer cells were treated for 48 hours with SSALPs containing sphingomyelin(d18:1/18:1)-deuterium(d)₉. The biochemical fate of this compound was traced using liquid chromatography mass spectrometry (FIG. 7A). The ceramide(18:1/18:1)-d₉ isotopologue was detected in all three cell lines, whereas the glucosylceramide(18:1/18:1)-d₉ isotopologue was only detected in PC-3M and RM-9 prostate cancer cell lines. Notably, the ratio of ceramide(18:1/18:1)-d₉ to sphingomyelin(18:1/18:1)-d₉, based on peak area, was appreciably higher in PC-3M (ratio: 0.14) and RM-9 (ratio: 0.23) as compared to LNCaP (ratio: 0.02), demonstrating an overall higher metabolic flux into ceramide biosynthesis via sphingomyelin salvaging (FIG. 7B). Neither oleate-d9 nor ceramide(18:1/18:1-d9)-1-phosphate was observed, indicating preferential shunting of sphingomyelin-derived ceramides into the glycosphingolipid pathway rather than hydrolysis by ceramidases or phosphorylation. These findings give direct biochemical evidence that sphingomyelins are indeed a source of ceramides and their glycosylated derivatives (FIG. 4B).

### Example 9: Facilitation of Mitochondrial Components by Cav-1

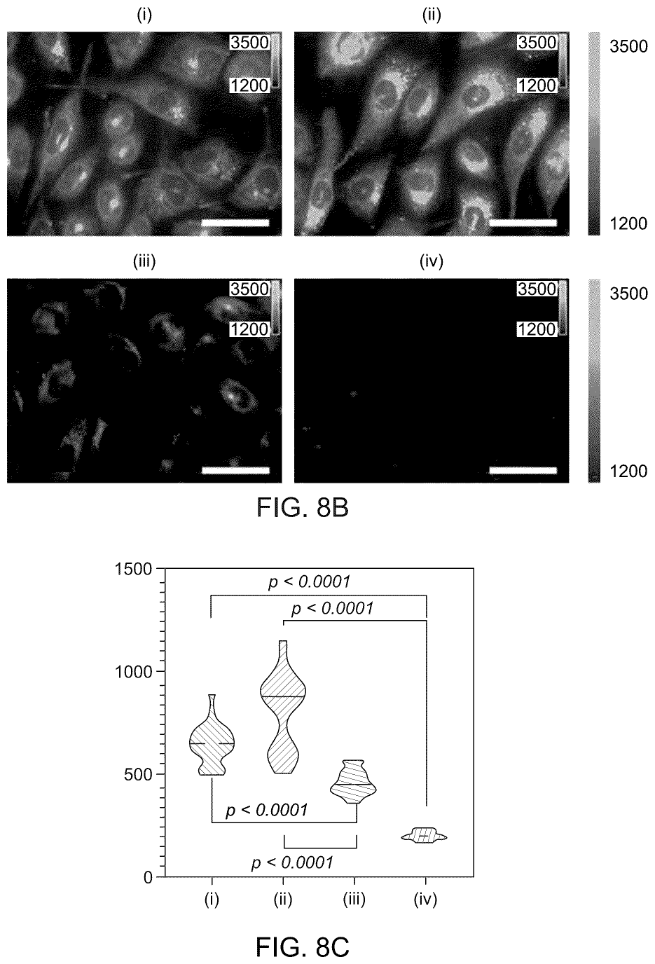

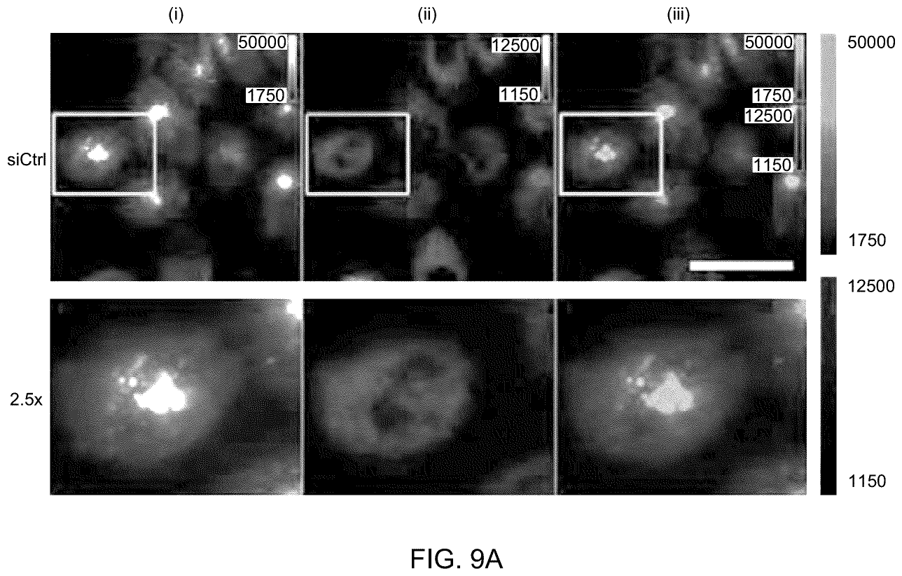

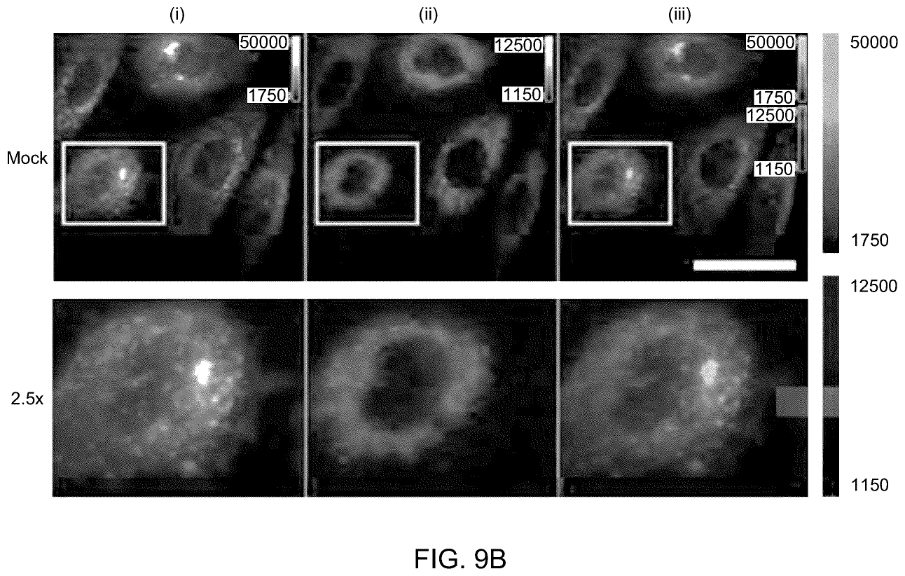

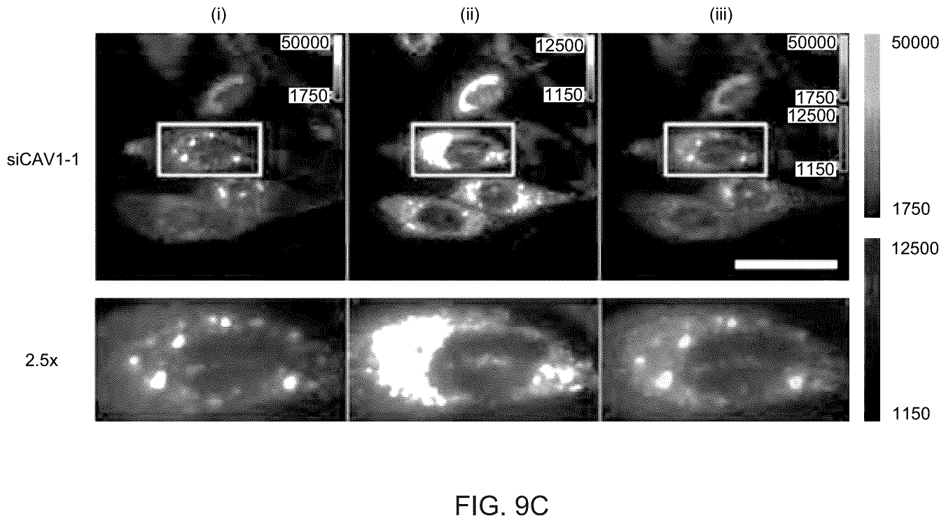

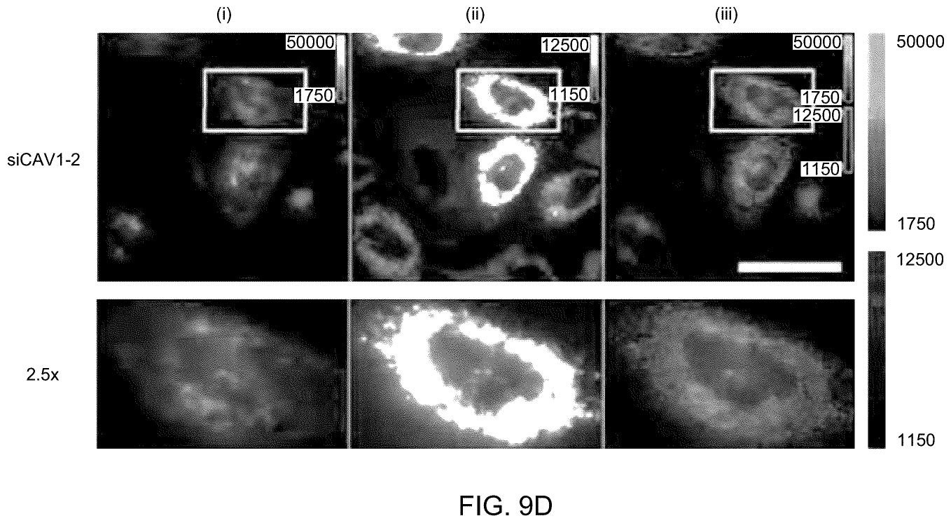

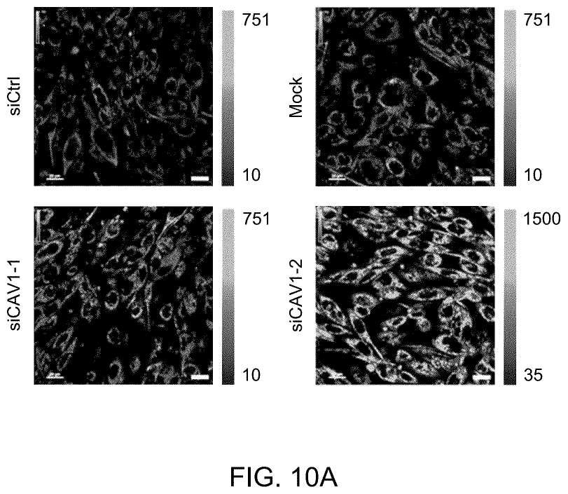

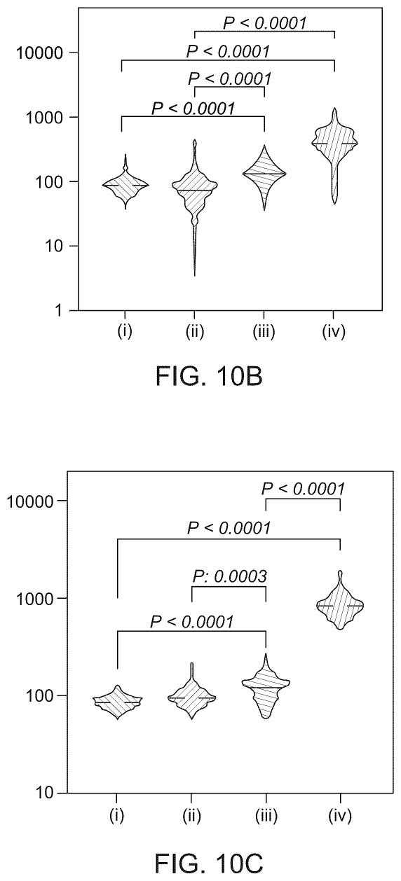

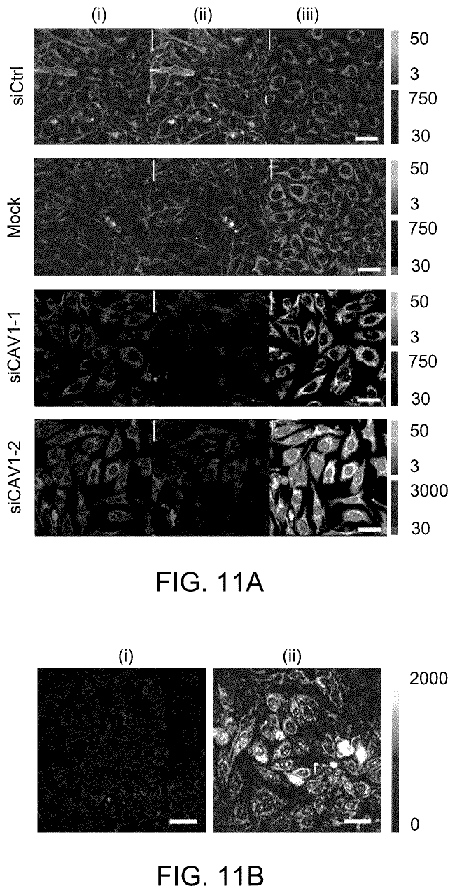

The findings above suggest a Cav-1-associated mechanistic framework that directs ceramide pools into glycosphingolipids, consistent with the observation that glycosphingolipids, particularly lactosylceramides, are key features of the plasma sphingolipid signature (FIG. 1B). Ceramides are bioactive sphingolipids that are actively involved in mediating cell death, including induction of apoptosis through cytochrome c release from the mitochondria, as well as targeting of mitochondria to autophagosomes to elicit lethal mitophagy. An association between Cav-1 and mitochondrial morphology was next probed in both PC-3M (Cav-1 high) and LNCaP (Cav-1 low) cell lines. PC-3M cells exhibited a more branched, fused-like-mitochondrial architecture with diffuse lysosomal staining, whereas the morphology of the mitochondria and lysosomes were more punctate in LNCaP cells (FIG. 8A). Using fluorescent-labeled SSALPs containing C11 TopFluor-SM, differential trafficking of sphingomyelin in PC-3M cells following knockdown of CAV1 was assessed. CAV1 knockdown resulted in statistically significantly (Tukey multiple comparison test 2-sided adjusted p<0.001) reduced uptake of the C11 TopFluor-SM-containing SSALPs (FIGS. 8B and 8D). Remarkably, knockdown of CAV1 in PC-3M also resulted in the accumulation of punctate mitochondria (FIGS. 9A, 9B, 9C, and 9D; FIGS. 10A and 10B) and a reduction in prevalence of lysosomes (FIGS. 9A, 9B, 9C, and 9D); these changes were accompanied by increases (Tukey multiple comparison test 2-sided adjusted p<0.001) in reactive oxygen species as assessed by MitoTracker Red CMXRos (Poot 1996) and CellROX Deep red (FIGS. 11A and 11B, FIG. 10C).

### Example 10: Release of Cav-1-Sphingomyelin/Lactosylceramide-Enriched EVs

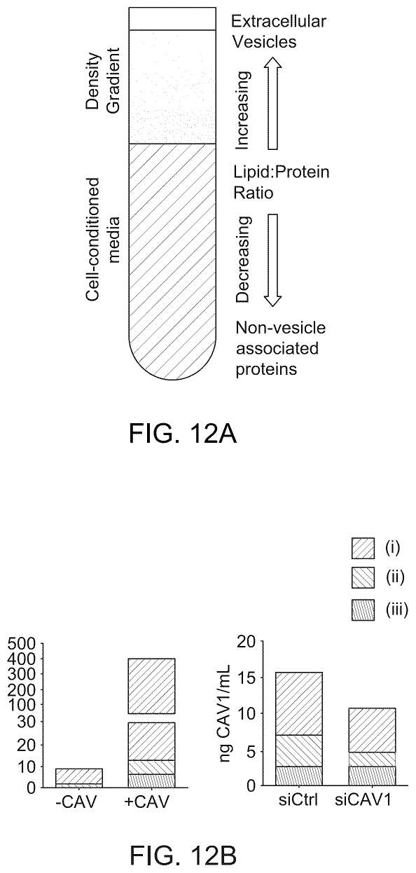

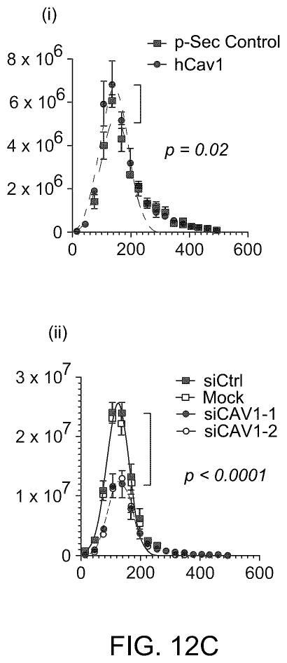

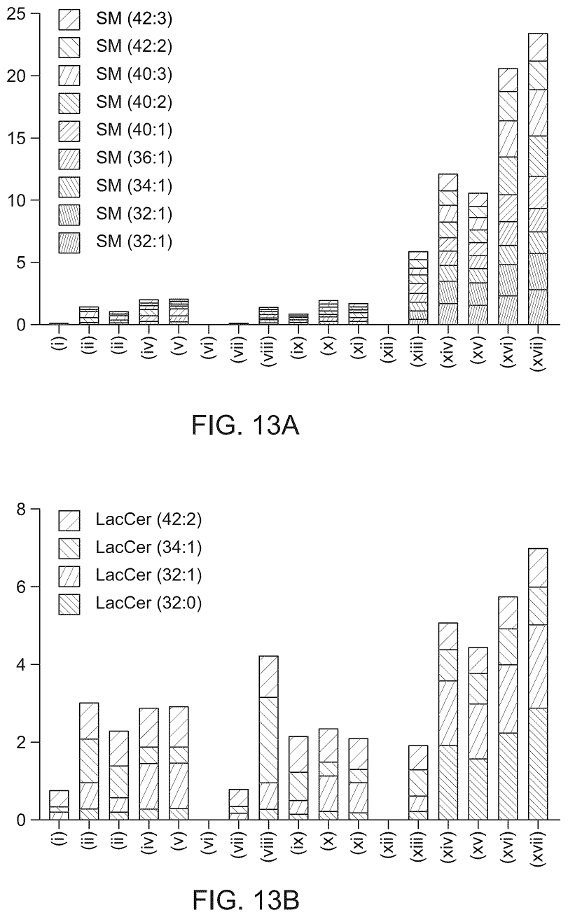

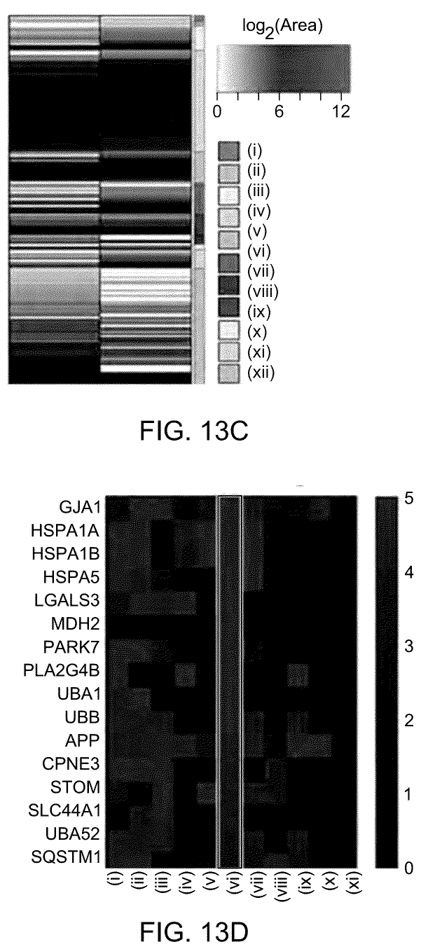

It was previously demonstrated that secretion of membrane associated Cav-1 by prostate cancer cells has been previously demonstrated. Consistent with this previous report, assessment of CM confirmed detectable levels of Cav-1 in CM from PC-3M and RM-9 prostate cancer cell lines but not LNCaP. Isolation of extracellular lipid vesicles (EVs) from CM of LNCaP and PC-3M following respective overexpression of Cav-1, or knockdown of CAV1, confirmed that Cav-1 is present on EVs (FIG. 8D and FIGS. 12A and 12B). Notably, the amount of Cav-1-containing EVs was appreciably higher when culture media was supplemented with exogenous low-density lipoproteins (FIG. 12B), consistent with the earlier observations that lysate Cav-1 protein expression is responsive to extracellular lipid availability (FIG. 3A). Concordantly, in comparison to respective controls, the concentration of EV particles in CM was statistically significantly higher (comparison of area under the curve 2-sided student t-test p: 0.02) in LNCaP following Cav-1 overexpression whereas the number of EVs in CM from PC-3M following knockdown of CAV1 was statistically significantly reduced (comparison of area under the curve 2-sided student t-test p<0.001) (FIGS. 12C and 12D). Moreover, it was determined from using density gradient fractionation that Cav-1 containing EVs were most highly enriched in a fraction within the 1.06-1.15 g mL⁻¹ buoyant density range of plasma high-density lipoproteins, indicating that secreted Cav-1 exists in an HDL-like lipid-protein particle. Analysis of the CM lipidome of LNCaP and PC-3M following Cav-1 overexpression or CAV1 knockdown, respectively, also indicated increased relative abundances of sphingomyelins and lactosylceramides that were dependent on Cav-1 and extracellular lipid bioavailability (FIGS. 13A and 13B).

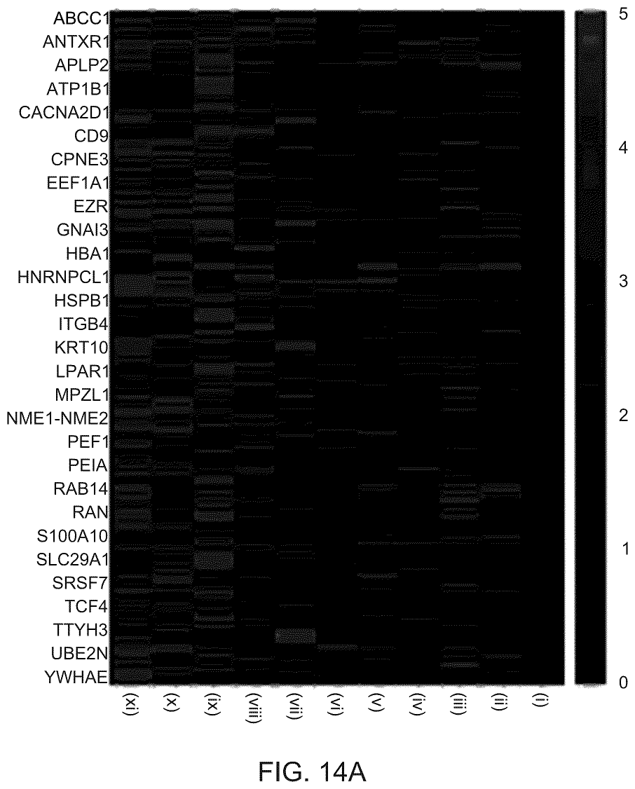

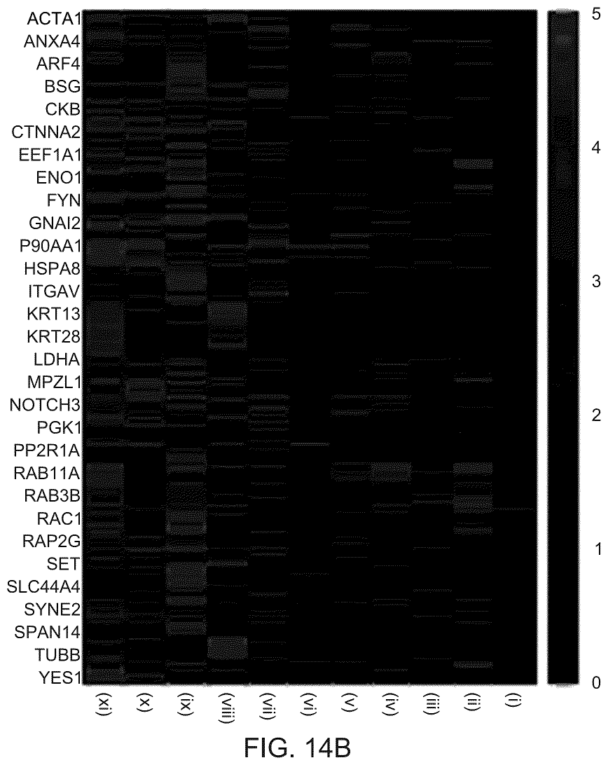

To determine the lipid composition and protein cargo of EVs, lipidomic and proteomic analyses were performed using mass spectrometry on EVs derived from LNCaP and PC-3M, respectively. Consistent with findings by others, analyses of the EV-lipidome identified sphingolipids as well as phosphatidylcholines to be particularly enriched. Interestingly cardiolipins, important lipid constituents of the inner mitochondrial membrane, were found to be present in prostate cancer cell line-derived EVs. Evaluation of the EV-proteome identified 237 and 341 (>5 spectral abundance) high confidence proteins in LNCaP and PC-3M derived EVs, respectively. To investigate for functional aspects of protein features, performed subcellular localization analysis, based on the COMPARTMENTS localization evidence database scores [doi.org/10.1093/database/bauOl2], were performed, filtering for genes confidently assigned to at least one ofthe 11 subcellular localizations (confidence score c2): nucleus, cytosol, cytoskeleton, peroxisome, lysosome, endoplasmic reticulum, Golgi apparatus, plasma membrane, endosome, extracellular spaces and mitochondrion (FIGS. 14A and 14). Subcellular localization analyses of EV-derived protein features evidenced representation ofproteins annotated as localized to the mitochondria (FIG. 13C; FIGS. 14A and 141B). IPA analysis of the 341 protein features detected in PC-3M-derived EVs revealed caveoloar-mediated endocytosis and phagosome maturation as a top network, and reduced apoptosis and necrosis and increased cell movement, degranulation and cell proliferation were predicted as top activated disease functions (Tables 8 and 9).

TABLE 8
Disease Function

| | adjusted P- | Predicted | |
|---|---|---|---|
| Disease function | value | activation state | # molecules |
| Cell movement | 2.09E−30 | Increased | 125 |
| Apoptosis | 2.8E−22 | Decreased | 119 |
| Necrosis | 1.06E−27 | Decreased | 134 |
| Degranulation of cells | 2.51E−31 | Increased | 69 |
| Cell proliferation of | 8.87E−20 | Increased | 112 |
| tumor cell lines | | | |

TABLE 9
Ingenuity canonical pathways

| | −log(p- | | # |
|---|---|---|---|
| Ingenuity canonical pathways | value) | Ratio | molecules |
| Virus entry via endocytic pathways | 27.6 | 0.28 | 30/107 |
| Caveolar-mediated endocytosis signaling | 21.1 | 0.301 | 22/73 |
| Phagosome maturation | 20.2 | 0.193 | 27/140 |
| Remodeling of epithelial adherens | 19.3 | 0.303 | 20/66 |
| junctions | | | |
| Germ cell-sertoli cell junction signaling | 18.2 | 0.163 | 27/166 |
| 14-3-3-mediated signaling | 17.9 | 0.190 | 24/126 |
| Ephrin receptor signaling | 17.4 | 0.152 | 27/178 |
| Axonal guidance signaling | 17.2 | 0.0878 | 41/467 |
| Epithelial adherens junction signaling | 17.1 | 0.167 | 25/150 |
| Clathrin-mediated endocytosis signaling | 16.5 | 0.141 | 27/192 |

Collectively, these findings indicate that prostate cancer cells robustly secrete Cav-1-containing sphingolipid-enriched EVs that are enriched in a diverse repertoire of proteins including mitochondrial-associated proteins and lipids. On the basis of these findings, Cav-1 mediated uptake of sphingomyelin (FIGS. 8B and 8C), conversion of sphingomyelin to ceramide and its subsequent glycosphingolipid derivatives (FIG. 7B), and inclusion of mitochondrial proteins in the EV cargo (FIG. 12C, FIG. 12D, and FIGS. 14A and 14B) is apparently associated with clearance of mitochondria components.

### Example 11: Targeting of the Shunt from Ceramides to Glycosphingolipids

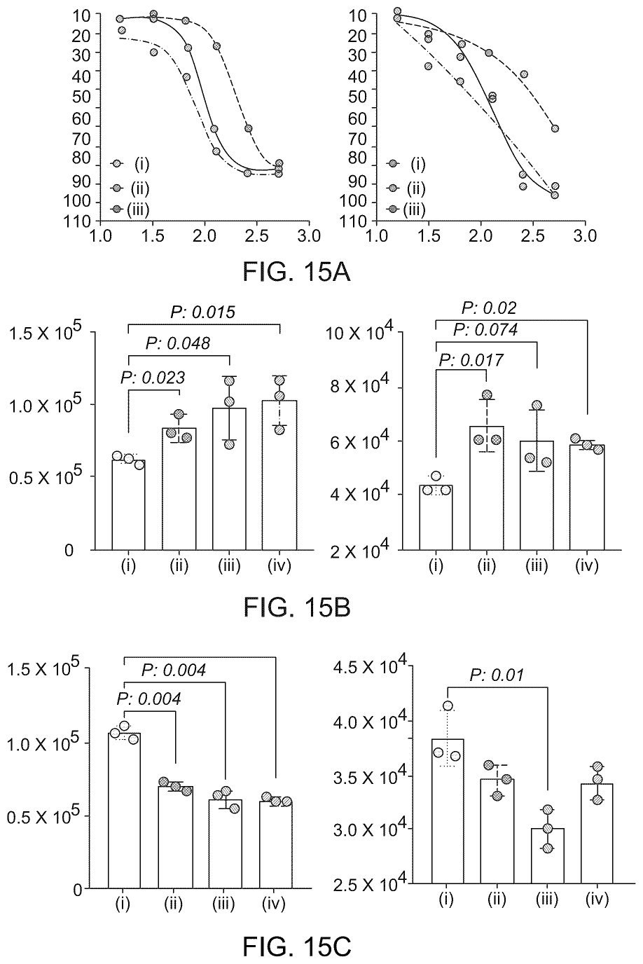

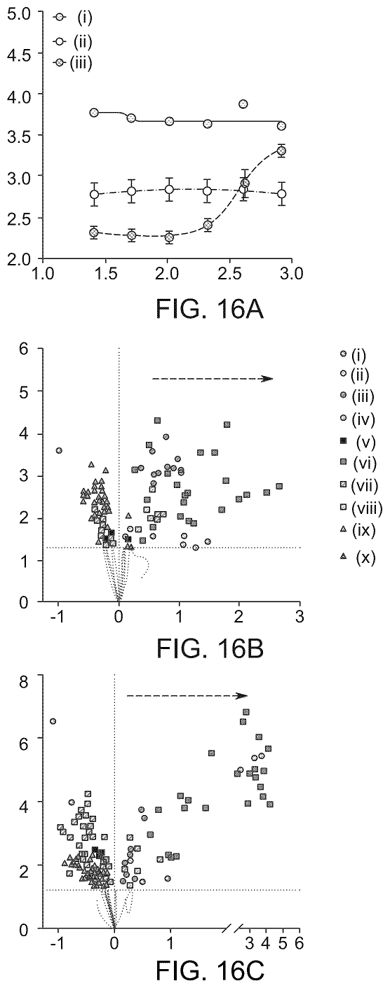

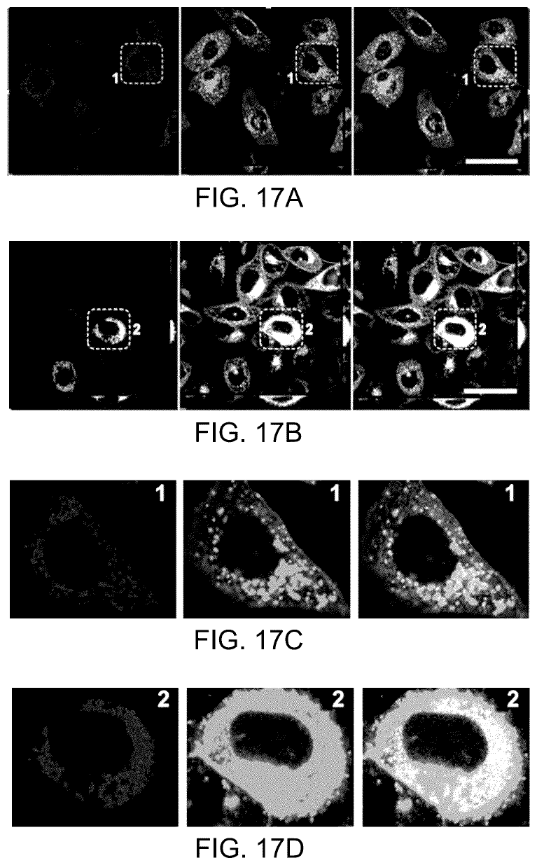

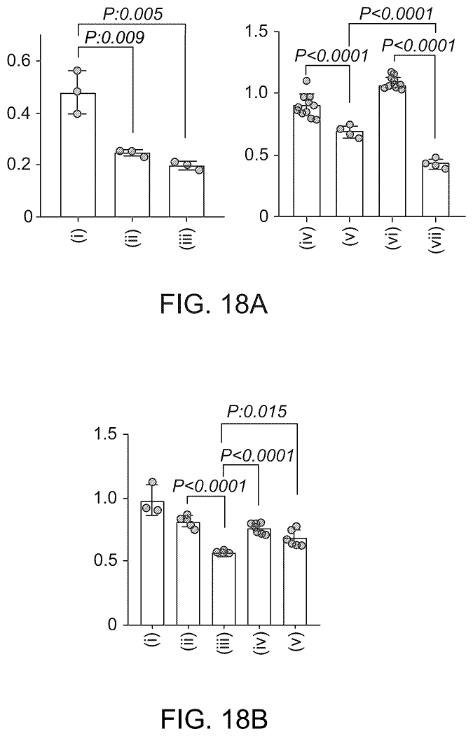

These findings of an adaptive Cav-1-mediated glycosphingolipid mechanism suggest that that targeting of the ceramide to glycosphingolipid conversion may represent an actionable metabolic vulnerability in prostate cancer. To test this hypothesis, the efficacy of PDMP, PPMP and eliglustat three different inhibitors of glucosylceramide synthase (also known as UGCG; UDP-glucose:ceramide glucosyltransferase), a rate-limiting enzyme in glycosphingolipid metabolism, for reducing viability of RM-9 and PC-3M prostate cancer cells in vitro was then evaluated. Treatment of RM-9 and PC-3M prostate cancer cells with PDMP, PPMP and eliglustat resulted in dose-dependent cytotoxicity (FIGS. 15A, 15B, and 15C). Next, alterations in the lipidome following pharmacological inhibition of UGCG in RM-9 and PC-3M prostate cancer cells were evaluated. RM-9 and PC-3M cells were challenged with PDMP, PPMP, eliglustat or vehicle and evaluated after six hours of treatment to capture early metabolic changes, particularly in sphingolipid metabolism, and to mitigate influence of secondary events that may cause elevations in ceramide pools, and reductions in GLS expression that can occur as a result of reduced cell survival. Short-term (6 hr) challenge of RM-9 and PC-3M prostate cells with PDMP, PPMP or eliglustat resulted in accumulation of intracellular ceramides, acylcarnitines, lysophospholipids and diacylglycerols and reductions in glycosphingolipids, phospholipids and triacylglycerols (FIGS. 15A, 15B, and 15C, FIGS. 16A, 16B, and 16C). Notably, the acute cytotoxic effects of eliglustat were mediated through a non-apoptotic mechanism (FIG. 16A). Reductions in phospholipids and triacylglycerols coupled with elevations in their downstream catabolites suggested mitophagy. Consistent with this, treatment of PC-3M cells with eliglustat increased protein expression of the mitophagy-associated markers LC3B-II (FIGS. 14A and 14B). Assessment of mitochondrial morphology in PC-3M cells following acute (6 hr) treatment with eliglustat indicated loss of the branched, fused-like-mitochondrial architecture and accumulation of punctate mitochondria co-localized with the lysosome (FIGS. 17A, 17B, 17C, and 17D); these changes were met with increased protein expression of Parkin and PINK1, further suggesting increased mitophagy. Previous reports have demonstrated that ceramides target autophagosomes to mitochondria to induce lethal mitophagy. Notably, knockdown of CAV1 or pretreatment with a Cav-1 specific monoclonal antibody (Cav-1mAb) further sensitized PC-3M cells to eliglustat whereas overexpression of Cav-1 in LNCaP reduced the anti-cancer effects of eliglustat (FIGS. 18A and 18B).

### Example 12: Inhibition of RM-9 Tumor Growth by Eliglustat

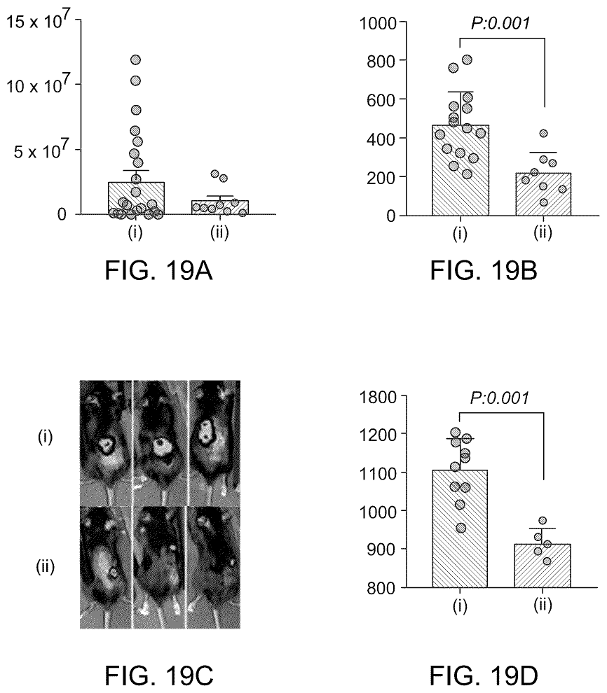

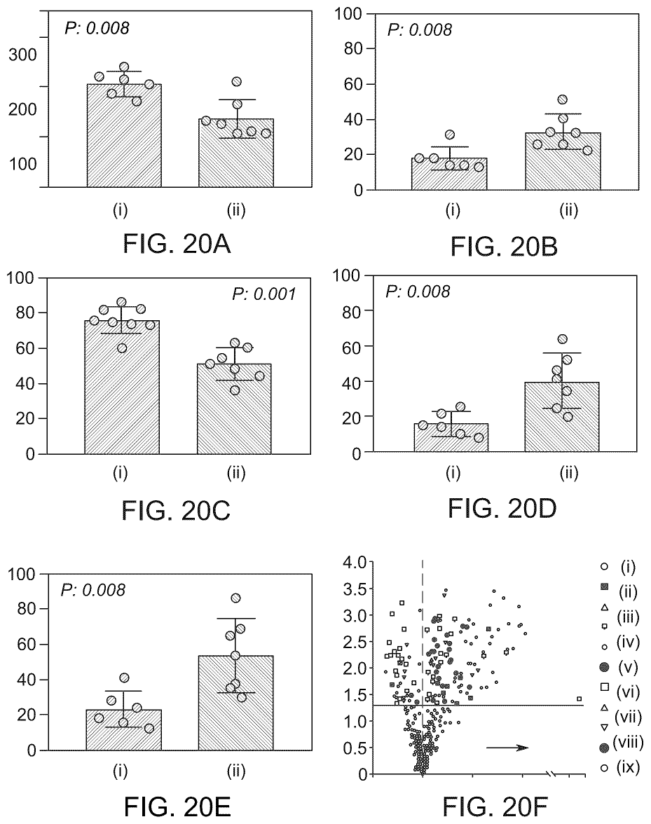

Inhibition of tumor growth by eliglustat was next examined. RM-9 cells harbor driver oncogenic RAS and MYC genes which model RAS-MAPK pathway activation and MYC-driven transcriptional activities which are associated with aggressive primary prostate cancer. RM-9-luciferase cells were subcutaneously implanted into C57BL/6N mice. RM-9 tumor growth was suppressed by eliglustat (FIGS. 19A, 19B, and 19C). Metabolomics analysis of tumor tissues from all treatment groups showed that eliglustat was linked to reductions in glycosphingolipids in RM-9 tumor bearing mice (FIG. 19D). Immunohistochemical analyses of tumor tissue indicated that treatment with eliglustat statistically significantly reduced Cav-1 and PCNA staining (2-sided Wilcoxon rank sum test p: 0.008 and 0.001, respectively), whereas BrdU-TUNEL and mitophagy-associated marker (LC3B and HMGB1) staining was statistically significantly increased (2-sided Wilcoxon rank sum test p: 0.008 for all three markers) (FIGS. 20A, 20B, 20C, 20D, 20E, and 20F). Notably, these results indicated that RM-9 tumors were associated with a plasma lipid signature, similar to that observed in prostate cancer patients, that included elevations in several sphingomyelins and glycosphingolipids (FIGS. 20A, 20B, 20C, 20D, 20E, and 20F). In addition, plasma Cav-1 levels tended to be elevated in RM-9 tumor-bearing mice compared to control mice. Interestingly, plasma levels of Cav-1 and identified lipid species that are part of the Cav-1 sphingolipid signature increased upon treatment with eliglustat in RM-9 tumor bearing mice. Treatment with eliglustat in RM-9 tumor bearing mice also resulted in statistically significant increases (2-sided Wilcoxon rank sum test p: 0.004) in plasma ceramides, suggesting that the elevation in plasma Cav-1 and sphingolipids may be a consequence of cell death (FIGS. 19A, 19B, and 19C).

### Example 13: Calculation of Biomarker Score

Measured concentrations of plasma Cav-1-sphingolipid signature features were used to calculate a biomarker score based on a logistic regression model. In this model for progression of prostate cancer, the values for the plasma analyte signature features are combined into a model that is applied to predict risk of disease progression.

The table below illustrates the ratio between the actual hazard and the baseline hazard rate as well as the hazard rate at different cutoff points of the biomarker panel score. Here, the hazard is defined as the risk of an event (i.e., disease progression) as a function of time where a hazard rate>than 1 implies that the time-to-disease progression is shorter.

The table shows two things as a function of model score. The first is the risk of progression relative to the calculated “baseline hazard rate” for a given signature, and the second considers the use of the signature score as a classifier cut point; the last column then gives the relative risk of disease progression between the high and low scoring groups, ie the “hazard ratio.”

The first column of the table shows the biomarker panel score in the Active Surveillance cohort, the second column describes the change in the actual hazard relative to the baseline hazard, and the third column illustrates the hazard ratio as well as the corresponding 95% confidence interval, based on a dichotomization of the population using different cutoff points for the biomarker panel score.

| | Ratio between the actual hazard rate | |
|---|---|---|
| Model | and the baseline hazard rate | Hazard Ratio (binary |
| Score | (h(t)/h0(t)) | cutoff) |
| 2.524 | 8.684 | |
| 2.718 | 10.252 | |
| 2.840 | 11.381 | |
| 3.017 | 13.251 | |
| 3.019 | 13.273 | |
| 3.056 | 13.700 | |
| 3.081 | 13.990 | |
| 3.110 | 14.347 | |
| 3.133 | 14.631 | |
| 3.157 | 14.934 | |
| 3.161 | 14.986 | 2.64 [0.369-18.969] |
| 3.162 | 15.004 | 2.76 [0.385-19.807] |
| 3.164 | 15.019 | 2.89 [0.402-20.694] |
| 3.166 | 15.048 | 3 [0.419-21.541] |
| 3.170 | 15.106 | 3.35 [0.467-23.999] |
| 3.171 | 15.114 | 3.47 [0.483-24.854] |
| 3.171 | 15.121 | 1.79 [0.441-7.266] |
| 3.175 | 15.163 | 1.25 [0.397-3.958] |
| 3.178 | 15.206 | 1.34 [0.423-4.223] |
| 3.182 | 15.261 | 1.47 [0.464-4.626] |
| 3.183 | 15.274 | 1.56 [0.493-4.918] |
| 3.188 | 15.336 | 1.6 [0.506-5.044] |
| 3.189 | 15.345 | 1.64 [0.519-5.178] |
| 3.202 | 15.520 | 1.68 [0.532-5.305] |
| 3.205 | 15.567 | 1.74 [0.551-5.498] |
| 3.208 | 15.604 | 1.86 [0.589-5.867] |
| 3.209 | 15.610 | 1.96 [0.621-6.187] |
| 3.227 | 15.863 | 2.06 [0.654-6.518] |
| 3.237 | 15.992 | 2.2 [0.696-6.939] |
| 3.247 | 16.127 | 2.26 [0.717-7.142] |
| 3.251 | 16.192 | 2.38 [0.755-7.524] |
| 3.262 | 16.338 | 2.43 [0.768-7.656] |
| 3.263 | 16.358 | 2.56 [0.812-8.088] |
| 3.264 | 16.364 | 2.63 [0.832-8.295] |
| 3.271 | 16.468 | 2.72 [0.861-8.582] |
| 3.279 | 16.581 | 2.83 [0.895-8.918] |
| 3.283 | 16.630 | 2.94 [0.93-9.267] |
| 3.287 | 16.689 | 2.98 [0.944-9.411] |
| 3.295 | 16.806 | 3.11 [0.984-9.81] |
| 3.298 | 16.855 | 3.22 [1.02-10.165] |
| 3.299 | 16.865 | 3.36 [1.066-10.617] |
| 3.304 | 16.942 | 2.58 [0.947-7.009] |
| 3.305 | 16.945 | 2.67 [0.982-7.272] |
| 3.312 | 17.051 | 2.13 [0.865-5.235] |
| 3.317 | 17.130 | 2.19 [0.89-5.387] |
| 3.324 | 17.235 | 2.22 [0.902-5.456] |
| 3.328 | 17.295 | 1.87 [0.818-4.27] |
| 3.333 | 17.361 | 1.59 [0.739-3.439] |
| 3.333 | 17.366 | 1.64 [0.76-3.54] |
| 3.333 | 17.371 | 1.7 [0.786-3.659] |
| 3.338 | 17.432 | 1.49 [0.722-3.069] |
| 3.338 | 17.440 | 1.51 [0.73-3.104] |
| 3.357 | 17.720 | 1.52 [0.738-3.139] |
| 3.358 | 17.745 | 1.55 [0.751-3.191] |
| 3.366 | 17.856 | 1.61 [0.783-3.325] |
| 3.376 | 18.009 | 1.63 [0.791-3.359] |
| 3.380 | 18.082 | 1.65 [0.799-3.394] |
| 3.391 | 18.248 | 1.68 [0.817-3.469] |
| 3.394 | 18.293 | 1.74 [0.844-3.583] |
| 3.406 | 18.482 | 1.78 [0.861-3.659] |
| 3.407 | 18.504 | 1.8 [0.874-3.712] |
| 3.409 | 18.535 | 1.83 [0.887-3.768] |
| 3.426 | 18.810 | 1.83 [0.887-3.768] |
| 3.429 | 18.851 | 1.88 [0.915-3.885] |
| 3.431 | 18.888 | 1.69 [0.849-3.346] |
| 3.434 | 18.931 | 1.71 [0.861-3.392] |
| 3.436 | 18.971 | 1.74 [0.878-3.458] |
| 3.438 | 18.993 | 1.78 [0.894-3.525] |
| 3.440 | 19.027 | 1.81 [0.913-3.598] |
| 3.441 | 19.052 | 1.86 [0.939-3.7] |
| 3.443 | 19.078 | 1.69 [0.879-3.252] |
| 3.443 | 19.083 | 1.71 [0.89-3.294] |
| 3.446 | 19.131 | 1.75 [0.912-3.375] |
| 3.450 | 19.193 | 1.79 [0.928-3.434] |
| 3.462 | 19.390 | 1.82 [0.944-3.495] |
| 3.464 | 19.431 | 1.83 [0.952-3.523] |
| 3.469 | 19.511 | 1.85 [0.964-3.566] |
| 3.477 | 19.645 | 1.87 [0.971-3.595] |
| 3.482 | 19.728 | 1.89 [0.983-3.639] |
| 3.483 | 19.741 | 1.92 [1-3.701] |
| 3.487 | 19.818 | 1.96 [1.019-3.772] |
| 3.488 | 19.828 | 1.77 [0.945-3.313] |
| 3.488 | 19.830 | 1.8 [0.961-3.372] |
| 3.490 | 19.859 | 1.83 [0.98-3.436] |
| 3.494 | 19.933 | 1.89 [1.008-3.536] |
| 3.494 | 19.933 | 1.9 [1.016-3.562] |
| 3.500 | 20.034 | 1.75 [0.955-3.197] |
| 3.501 | 20.057 | 1.6 [0.892-2.868] |
| 3.504 | 20.106 | 1.61 [0.899-2.889] |
| 3.512 | 20.235 | 1.62 [0.905-2.91] |
| 3.513 | 20.256 | 1.63 [0.912-2.931] |
| 3.525 | 20.473 | 1.66 [0.927-2.98] |
| 3.526 | 20.493 | 1.7 [0.946-3.041] |
| 3.528 | 20.515 | 1.73 [0.963-3.096] |
| 3.530 | 20.553 | 1.6 [0.908-2.815] |
| 3.531 | 20.576 | 1.63 [0.926-2.872] |
| 3.532 | 20.583 | 1.66 [0.942-2.922] |
| 3.534 | 20.622 | 1.69 [0.958-2.971] |
| 3.535 | 20.651 | 1.72 [0.979-3.036] |
| 3.536 | 20.662 | 1.76 [0.998-3.094] |
| 3.538 | 20.697 | 1.77 [1.007-3.124] |
| 3.539 | 20.722 | 1.81 [1.027-3.184] |
| 3.543 | 20.784 | 1.82 [1.033-3.204] |
| 3.545 | 20.816 | 1.86 [1.055-3.271] |
| 3.549 | 20.886 | 1.9 [1.077-3.339] |
| 3.556 | 21.012 | 1.93 [1.099-3.407] |
| 3.558 | 21.047 | 1.97 [1.121-3.477] |
| 3.558 | 21.054 | 2 [1.138-3.53] |
| 3.559 | 21.069 | 2.02 [1.145-3.552] |
| 3.569 | 21.249 | 2.04 [1.156-3.584] |
| 3.577 | 21.402 | 2.05 [1.163-3.606] |
| 3.577 | 21.404 | 2.06 [1.17-3.628] |
| 3.586 | 21.562 | 2.1 [1.19-3.691] |
| 3.589 | 21.617 | 2.11 [1.197-3.713] |
| 3.589 | 21.624 | 2.14 [1.214-3.765] |
| 3.594 | 21.704 | 2.16 [1.225-3.798] |
| 3.597 | 21.771 | 2.19 [1.246-3.864] |
| 3.598 | 21.788 | 2.03 [1.171-3.517] |
| 3.600 | 21.831 | 2.05 [1.181-3.548] |
| 3.600 | 21.834 | 2.09 [1.203-3.616] |
| 3.601 | 21.846 | 2.12 [1.224-3.676] |
| 3.608 | 21.967 | 2.15 [1.24-3.725] |
| 3.608 | 21.967 | 2.18 [1.257-3.778] |
| 3.614 | 22.079 | 2.18 [1.257-3.778] |
| 3.619 | 22.181 | 2.04 [1.194-3.486] |
| 3.620 | 22.196 | 2.07 [1.21-3.535] |
| 3.626 | 22.307 | 2.11 [1.233-3.6] |
| 3.626 | 22.308 | 2.12 [1.243-3.63] |
| 3.629 | 22.365 | 2.16 [1.266-3.697] |
| 3.632 | 22.430 | 2.19 [1.283-3.747] |
| 3.633 | 22.446 | 2.22 [1.301-3.8] |
| 3.635 | 22.485 | 2.26 [1.324-3.868] |
| 3.637 | 22.530 | 2.3 [1.345-3.93] |
| 3.638 | 22.542 | 2.32 [1.356-3.962] |
| 3.640 | 22.586 | 2.33 [1.364-3.984] |
| 3.641 | 22.597 | 2.37 [1.385-4.046] |
| 3.644 | 22.664 | 2.21 [1.308-3.723] |
| 3.650 | 22.783 | 2.06 [1.237-3.442] |
| 3.651 | 22.790 | 2.09 [1.253-3.487] |
| 3.659 | 22.952 | 2.12 [1.269-3.53] |
| 3.659 | 22.957 | 1.98 [1.203-3.276] |
| 3.664 | 23.053 | 2.02 [1.223-3.333] |
| 3.665 | 23.076 | 2.03 [1.23-3.351] |
| 3.667 | 23.110 | 2.04 [1.236-3.368] |
| 3.667 | 23.117 | 2.06 [1.25-3.406] |
| 3.667 | 23.117 | 2.09 [1.269-3.458] |
| 3.669 | 23.144 | 2.13 [1.288-3.51] |
| 3.672 | 23.206 | 2.15 [1.302-3.548] |
| 3.672 | 23.212 | 2.16 [1.309-3.566] |
| 3.672 | 23.216 | 2.03 [1.242-3.321] |
| 3.673 | 23.235 | 2.04 [1.249-3.338] |
| 3.675 | 23.274 | 2.07 [1.267-3.388] |
| 3.680 | 23.368 | 2.08 [1.274-3.405] |
| 3.683 | 23.438 | 1.96 [1.211-3.181] |
| 3.683 | 23.442 | 1.85 [1.153-2.98] |
| 3.685 | 23.468 | 1.87 [1.161-3.002] |
| 3.685 | 23.471 | 1.78 [1.115-2.839] |
| 3.688 | 23.531 | 1.79 [1.12-2.854] |
| 3.697 | 23.715 | 1.8 [1.126-2.868] |
| 3.697 | 23.722 | 1.81 [1.131-2.882] |
| 3.707 | 23.917 | 1.71 [1.08-2.713] |
| 3.708 | 23.937 | 1.74 [1.097-2.758] |
| 3.711 | 23.995 | 1.74 [1.097-2.758] |
| 3.714 | 24.059 | 1.66 [1.052-2.612] |
| 3.716 | 24.100 | 1.67 [1.058-2.626] |
| 3.716 | 24.110 | 1.69 [1.071-2.658] |
| 3.717 | 24.117 | 1.6 [1.024-2.513] |
| 3.725 | 24.298 | 1.55 [0.996-2.418] |
| 3.729 | 24.377 | 1.56 [1.004-2.437] |
| 3.731 | 24.424 | 1.57 [1.009-2.449] |
| 3.733 | 24.447 | 1.59 [1.021-2.479] |
| 3.734 | 24.484 | 1.61 [1.032-2.505] |
| 3.735 | 24.502 | 1.63 [1.047-2.541] |
| 3.736 | 24.523 | 1.55 [1.002-2.409] |
| 3.737 | 24.540 | 1.48 [0.961-2.288] |
| 3.737 | 24.546 | 1.49 [0.965-2.299] |
| 3.738 | 24.565 | 1.51 [0.981-2.336] |
| 3.742 | 24.656 | 1.53 [0.991-2.36] |
| 3.744 | 24.687 | 1.55 [1.005-2.393] |
| 3.748 | 24.778 | 1.48 [0.963-2.276] |
| 3.750 | 24.818 | 1.49 [0.97-2.292] |
| 3.751 | 24.836 | 1.52 [0.988-2.335] |
| 3.751 | 24.840 | 1.54 [1.001-2.364] |
| 3.756 | 24.944 | 1.57 [1.019-2.408] |
| 3.761 | 25.052 | 1.57 [1.019-2.408] |
| 3.764 | 25.116 | 1.59 [1.034-2.442] |
| 3.766 | 25.154 | 1.61 [1.046-2.47] |
| 3.767 | 25.188 | 1.63 [1.062-2.509] |
| 3.772 | 25.281 | 1.64 [1.07-2.527] |
| 3.774 | 25.334 | 1.66 [1.077-2.545] |
| 3.775 | 25.351 | 1.68 [1.09-2.575] |
| 3.776 | 25.365 | 1.69 [1.098-2.594] |
| 3.777 | 25.388 | 1.71 [1.111-2.625] |
| 3.779 | 25.442 | 1.65 [1.076-2.523] |
| 3.780 | 25.457 | 1.66 [1.087-2.548] |
| 3.782 | 25.509 | 1.68 [1.095-2.566] |
| 3.785 | 25.578 | 1.6 [1.051-2.445] |
| 3.787 | 25.611 | 1.53 [1.009-2.333] |
| 3.788 | 25.630 | 1.47 [0.969-2.228] |
| 3.790 | 25.686 | 1.41 [0.932-2.13] |
| 3.790 | 25.692 | 1.42 [0.938-2.144] |
| 3.792 | 25.729 | 1.44 [0.955-2.184] |
| 3.792 | 25.736 | 1.45 [0.96-2.194] |
| 3.797 | 25.841 | 1.47 [0.969-2.216] |
| 3.798 | 25.850 | 1.48 [0.981-2.241] |
| 3.799 | 25.880 | 1.49 [0.987-2.257] |
| 3.799 | 25.880 | 1.43 [0.949-2.159] |
| 3.801 | 25.928 | 1.45 [0.964-2.192] |
| 3.803 | 25.968 | 1.46 [0.97-2.207] |
| 3.806 | 26.025 | 1.47 [0.975-2.218] |
| 3.811 | 26.147 | 1.48 [0.982-2.233] |
| 3.811 | 26.154 | 1.49 [0.986-2.244] |
| 3.812 | 26.163 | 1.51 [1-2.274] |
| 3.814 | 26.215 | 1.52 [1.005-2.285] |
| 3.815 | 26.231 | 1.54 [1.02-2.32] |
| 3.816 | 26.251 | 1.56 [1.031-2.346] |
| 3.819 | 26.319 | 1.5 [0.994-2.252] |
| 3.827 | 26.512 | 1.5 [0.999-2.262] |
| 3.828 | 26.542 | 1.52 [1.01-2.288] |
| 3.829 | 26.563 | 1.54 [1.022-2.314] |
| 3.831 | 26.597 | 1.56 [1.036-2.345] |
| 3.832 | 26.617 | 1.57 [1.046-2.368] |
| 3.833 | 26.644 | 1.59 [1.06-2.4] |
| 3.834 | 26.675 | 1.6 [1.065-2.411] |
| 3.836 | 26.709 | 1.61 [1.072-2.427] |
| 3.836 | 26.717 | 1.55 [1.035-2.333] |
| 3.838 | 26.752 | 1.57 [1.049-2.365] |
| 3.838 | 26.767 | 1.6 [1.063-2.397] |
| 3.839 | 26.777 | 1.6 [1.068-2.408] |
| 3.839 | 26.779 | 1.62 [1.08-2.436] |
| 3.839 | 26.785 | 1.65 [1.1-2.48] |
| 3.840 | 26.800 | 1.59 [1.058-2.377] |
| 3.844 | 26.893 | 1.6 [1.066-2.394] |
| 3.844 | 26.898 | 1.61 [1.076-2.417] |
| 3.846 | 26.951 | 1.63 [1.089-2.446] |
| 3.850 | 27.042 | 1.65 [1.101-2.475] |
| 3.852 | 27.070 | 1.67 [1.113-2.501] |
| 3.853 | 27.113 | 1.69 [1.128-2.535] |
| 3.855 | 27.147 | 1.62 [1.086-2.431] |
| 3.856 | 27.172 | 1.64 [1.098-2.459] |
| 3.857 | 27.189 | 1.67 [1.115-2.496] |
| 3.864 | 27.363 | 1.68 [1.12-2.508] |
| 3.866 | 27.409 | 1.7 [1.133-2.537] |
| 3.866 | 27.414 | 1.72 [1.146-2.567] |
| 3.868 | 27.447 | 1.65 [1.103-2.463] |
| 3.868 | 27.459 | 1.67 [1.115-2.489] |
| 3.869 | 27.486 | 1.68 [1.126-2.514] |
| 3.870 | 27.514 | 1.7 [1.137-2.538] |
| 3.871 | 27.531 | 1.72 [1.149-2.566] |
| 3.874 | 27.586 | 1.75 [1.171-2.614] |
| 3.875 | 27.631 | 1.76 [1.176-2.626] |
| 3.876 | 27.646 | 1.69 [1.132-2.521] |
| 3.879 | 27.722 | 1.72 [1.149-2.56] |
| 3.879 | 27.723 | 1.73 [1.161-2.586] |
| 3.881 | 27.766 | 1.75 [1.174-2.616] |
| 3.883 | 27.806 | 1.77 [1.188-2.646] |
| 3.889 | 27.947 | 1.8 [1.204-2.683] |
| 3.891 | 28.012 | 1.83 [1.223-2.725] |
| 3.893 | 28.039 | 1.84 [1.235-2.752] |
| 3.900 | 28.208 | 1.8 [1.204-2.678] |
| 3.902 | 28.255 | 1.81 [1.213-2.696] |
| 3.904 | 28.310 | 1.75 [1.172-2.6] |
| 3.912 | 28.504 | 1.76 [1.185-2.629] |
| 3.921 | 28.739 | 1.7 [1.14-2.526] |
| 3.923 | 28.782 | 1.71 [1.146-2.538] |
| 3.923 | 28.785 | 1.73 [1.164-2.578] |
| 3.925 | 28.823 | 1.74 [1.172-2.597] |
| 3.927 | 28.874 | 1.76 [1.18-2.615] |
| 3.927 | 28.875 | 1.78 [1.197-2.652] |
| 3.928 | 28.902 | 1.8 [1.209-2.679] |
| 3.928 | 28.903 | 1.81 [1.215-2.692] |
| 3.933 | 29.021 | 1.83 [1.229-2.722] |
| 3.934 | 29.043 | 1.85 [1.244-2.756] |
| 3.938 | 29.149 | 1.87 [1.258-2.787] |
| 3.947 | 29.370 | 1.89 [1.267-2.807] |
| 3.947 | 29.372 | 1.9 [1.276-2.827] |
| 3.948 | 29.394 | 1.85 [1.242-2.749] |
| 3.952 | 29.498 | 1.79 [1.206-2.666] |
| 3.952 | 29.513 | 1.74 [1.168-2.582] |
| 3.953 | 29.518 | 1.76 [1.185-2.619] |
| 3.954 | 29.564 | 1.78 [1.199-2.649] |
| 3.955 | 29.582 | 1.78 [1.199-2.649] |
| 3.958 | 29.645 | 1.81 [1.216-2.688] |
| 3.960 | 29.702 | 1.83 [1.23-2.719] |
| 3.963 | 29.791 | 1.86 [1.25-2.764] |
| 3.970 | 29.970 | 1.87 [1.26-2.784] |
| 3.972 | 30.004 | 1.9 [1.278-2.826] |
| 3.974 | 30.052 | 1.84 [1.239-2.737] |
| 3.975 | 30.101 | 1.78 [1.196-2.644] |
| 3.979 | 30.189 | 1.8 [1.209-2.673] |
| 3.980 | 30.223 | 1.82 [1.227-2.713] |
| 3.984 | 30.335 | 1.83 [1.234-2.726] |
| 3.990 | 30.483 | 1.86 [1.248-2.759] |
| 3.990 | 30.490 | 1.87 [1.258-2.78] |
| 3.991 | 30.508 | 1.89 [1.274-2.816] |
| 3.993 | 30.544 | 1.93 [1.296-2.864] |
| 3.993 | 30.553 | 1.86 [1.253-2.772] |
| 3.994 | 30.579 | 1.89 [1.272-2.814] |
| 3.996 | 30.643 | 1.9 [1.279-2.829] |
| 4.001 | 30.774 | 1.92 [1.289-2.851] |
| 4.003 | 30.817 | 1.93 [1.296-2.865] |
| 4.010 | 30.996 | 1.87 [1.255-2.778] |
| 4.013 | 31.084 | 1.9 [1.275-2.821] |
| 4.015 | 31.148 | 1.91 [1.285-2.844] |
| 4.025 | 31.411 | 1.94 [1.301-2.879] |
| 4.030 | 31.531 | 1.96 [1.318-2.918] |
| 4.031 | 31.572 | 1.89 [1.268-2.809] |
| 4.033 | 31.633 | 1.9 [1.274-2.824] |
| 4.042 | 31.853 | 1.91 [1.281-2.838] |
| 4.043 | 31.882 | 1.92 [1.292-2.861] |
| 4.046 | 31.966 | 1.93 [1.298-2.876] |
| 4.053 | 32.175 | 1.86 [1.248-2.769] |
| 4.054 | 32.190 | 1.88 [1.263-2.801] |
| 4.054 | 32.198 | 1.81 [1.214-2.697] |
| 4.056 | 32.239 | 1.83 [1.228-2.729] |
| 4.071 | 32.667 | 1.85 [1.238-2.751] |
| 4.076 | 32.820 | 1.88 [1.261-2.802] |
| 4.080 | 32.914 | 1.92 [1.288-2.864] |
| 4.082 | 32.985 | 1.93 [1.296-2.88] |
| 4.084 | 33.031 | 1.98 [1.325-2.946] |
| 4.090 | 33.214 | 2.01 [1.347-2.996] |
| 4.091 | 33.230 | 1.93 [1.294-2.885] |
| 4.092 | 33.250 | 1.87 [1.249-2.79] |
| 4.092 | 33.267 | 1.89 [1.264-2.824] |
| 4.093 | 33.284 | 1.92 [1.282-2.865] |
| 4.094 | 33.316 | 1.93 [1.29-2.881] |
| 4.095 | 33.349 | 1.94 [1.297-2.898] |
| 4.097 | 33.417 | 1.88 [1.255-2.812] |
| 4.099 | 33.449 | 1.89 [1.263-2.828] |
| 4.101 | 33.529 | 1.94 [1.293-2.897] |
| 4.112 | 33.833 | 1.97 [1.313-2.942] |
| 4.117 | 33.990 | 1.89 [1.26-2.832] |
| 4.118 | 34.006 | 1.81 [1.208-2.726] |
| 4.121 | 34.102 | 1.85 [1.232-2.781] |
| 4.122 | 34.138 | 1.86 [1.239-2.798] |
| 4.126 | 34.234 | 1.87 [1.247-2.814] |
| 4.129 | 34.329 | 1.8 [1.195-2.708] |
| 4.130 | 34.375 | 1.83 [1.213-2.75] |
| 4.133 | 34.440 | 1.84 [1.221-2.766] |
| 4.133 | 34.447 | 1.86 [1.237-2.802] |
| 4.140 | 34.648 | 1.88 [1.248-2.828] |
| 4.144 | 34.790 | 1.92 [1.274-2.888] |
| 4.146 | 34.839 | 1.96 [1.301-2.95] |
| 4.153 | 35.039 | 1.97 [1.309-2.968] |
| 4.162 | 35.310 | 1.98 [1.317-2.987] |
| 4.162 | 35.316 | 2.02 [1.339-3.036] |
| 4.168 | 35.511 | 1.94 [1.283-2.922] |
| 4.173 | 35.659 | 1.96 [1.301-2.962] |
| 4.180 | 35.864 | 1.99 [1.319-3.003] |
| 4.187 | 36.076 | 2.02 [1.337-3.045] |
| 4.189 | 36.128 | 2.05 [1.356-3.088] |
| 4.191 | 36.215 | 2.06 [1.365-3.108] |
| 4.194 | 36.287 | 2.1 [1.392-3.17] |
| 4.196 | 36.347 | 2.15 [1.424-3.243] |
| 4.199 | 36.463 | 2.1 [1.385-3.171] |
| 4.202 | 36.557 | 2.15 [1.418-3.247] |
| 4.203 | 36.587 | 2.17 [1.432-3.281] |
| 4.208 | 36.750 | 2.18 [1.442-3.303] |
| 4.209 | 36.763 | 2.2 [1.453-3.326] |
| 4.209 | 36.770 | 2.21 [1.463-3.349] |
| 4.211 | 36.823 | 2.14 [1.413-3.253] |
| 4.213 | 36.887 | 2.16 [1.423-3.276] |
| 4.213 | 36.891 | 2.18 [1.439-3.312] |
| 4.220 | 37.130 | 2.22 [1.462-3.364] |
| 4.222 | 37.163 | 2.16 [1.421-3.291] |
| 4.224 | 37.238 | 2.2 [1.446-3.348] |
| 4.228 | 37.368 | 2.22 [1.457-3.373] |
| 4.236 | 37.642 | 2.29 [1.503-3.484] |
| 4.237 | 37.645 | 2.19 [1.436-3.349] |
| 4.241 | 37.779 | 2.25 [1.474-3.44] |
| 4.242 | 37.818 | 2.27 [1.486-3.468] |
| 4.245 | 37.925 | 2.3 [1.504-3.509] |
| 4.257 | 38.311 | 2.33 [1.522-3.552] |
| 4.279 | 39.042 | 2.37 [1.553-3.624] |
| 4.284 | 39.213 | 2.42 [1.585-3.698] |
| 4.284 | 39.218 | 2.48 [1.624-3.79] |
| 4.292 | 39.490 | 2.5 [1.637-3.821] |
| 4.299 | 39.702 | 2.53 [1.658-3.869] |
| 4.305 | 39.902 | 2.46 [1.606-3.775] |
| 4.308 | 40.011 | 2.49 [1.628-3.824] |
| 4.318 | 40.353 | 2.52 [1.642-3.857] |
| 4.322 | 40.487 | 2.56 [1.673-3.929] |
| 4.322 | 40.497 | 2.62 [1.707-4.01] |
| 4.331 | 40.802 | 2.54 [1.648-3.901] |
| 4.331 | 40.806 | 2.6 [1.687-3.993] |
| 4.332 | 40.865 | 2.48 [1.608-3.836] |
| 4.333 | 40.896 | 2.39 [1.541-3.71] |
| 4.357 | 41.732 | 2.49 [1.603-3.868] |
| 4.360 | 41.837 | 2.55 [1.642-3.962] |
| 4.361 | 41.880 | 2.6 [1.676-4.045] |
| 4.364 | 41.977 | 2.63 [1.694-4.087] |
| 4.369 | 42.178 | 2.71 [1.745-4.21] |
| 4.373 | 42.311 | 2.74 [1.762-4.252] |
| 4.375 | 42.385 | 2.67 [1.709-4.164] |
| 4.376 | 42.413 | 2.54 [1.622-3.993] |
| 4.380 | 42.551 | 2.6 [1.659-4.084] |
| 4.388 | 42.857 | 2.5 [1.585-3.946] |
| 4.389 | 42.894 | 2.38 [1.499-3.779] |
| 4.390 | 42.924 | 2.28 [1.428-3.646] |
| 4.395 | 43.113 | 2.35 [1.467-3.748] |
| 4.396 | 43.135 | 2.24 [1.395-3.613] |
| 4.400 | 43.306 | 2.32 [1.442-3.737] |
| 4.404 | 43.452 | 2.2 [1.355-3.567] |
| 4.410 | 43.677 | 2.11 [1.29-3.456] |
| 4.413 | 43.793 | 2.03 [1.231-3.36] |
| 4.414 | 43.839 | 1.96 [1.174-3.272] |
| 4.416 | 43.889 | 1.84 [1.089-3.105] |
| 4.434 | 44.575 | 1.86 [1.102-3.144] |
| 4.445 | 45.005 | 1.92 [1.139-3.248] |
| 4.450 | 45.186 | 1.96 [1.162-3.313] |
| 4.451 | 45.224 | 1.84 [1.073-3.139] |
| 4.462 | 45.645 | 1.89 [1.107-3.238] |
| 4.464 | 45.738 | 1.76 [1.017-3.061] |
| 4.471 | 46.007 | 1.66 [0.939-2.918] |
| 4.481 | 46.394 | 1.72 [0.978-3.039] |
| 4.481 | 46.416 | 1.76 [1-3.107] |
| 4.481 | 46.423 | 1.79 [1.015-3.152] |
| 4.487 | 46.634 | 1.79 [1.015-3.152] |
| 4.488 | 46.683 | 1.86 [1.053-3.272] |
| 4.509 | 47.533 | 1.94 [1.102-3.422] |
| 4.509 | 47.540 | 2.01 [1.14-3.54] |
| 4.517 | 47.877 | 2.09 [1.189-3.692] |
| 4.518 | 47.916 | 1.98 [1.105-3.558] |
| 4.525 | 48.202 | 2.02 [1.126-3.623] |
| 4.548 | 49.131 | 2.13 [1.186-3.817] |
| 4.552 | 49.304 | 2.17 [1.207-3.887] |
| 4.570 | 50.100 | 1.99 [1.089-3.651] |
| 4.573 | 50.230 | 1.84 [0.983-3.452] |
| 4.588 | 50.855 | 1.9 [1.012-3.553] |
| 4.603 | 51.541 | 1.93 [1.032-3.624] |
| 4.608 | 51.765 | 1.85 [0.963-3.57] |
| 4.609 | 51.769 | 1.96 [1.016-3.766] |
| 4.614 | 52.010 | 2.05 [1.066-3.951] |
| 4.628 | 52.631 | 2.2 [1.142-4.234] |
| 4.642 | 53.286 | 1.98 [0.997-3.935] |
| 4.645 | 53.400 | 2.03 [1.022-4.034] |
| 4.650 | 53.632 | 2.19 [1.103-4.354] |
| 4.652 | 53.732 | 2.34 [1.177-4.646] |
| 4.655 | 53.889 | 2.08 [1.011-4.3] |
| 4.658 | 54.006 | 2.18 [1.058-4.5] |
| 4.661 | 54.136 | 1.96 [0.91-4.24] |
| 4.691 | 55.559 | 1.69 [0.74-3.866] |
| 4.735 | 57.713 | 1.75 [0.765-3.994] |
| 4.776 | 59.768 | 1.89 [0.828-4.324] |
| 4.780 | 59.974 | 2.05 [0.896-4.676] |
| 4.839 | 63.081 | 2.17 [0.95-4.961] |
| 4.854 | 63.856 | 1.93 [0.786-4.758] |
| 4.861 | 64.239 | 2.21 [0.899-5.44] |
| 4.916 | 67.337 | 2.58 [1.049-6.348] |
| 4.968 | 70.427 | 2.94 [1.194-7.226] |
| 4.971 | 70.616 | 3.41 [1.385-8.385] |
| 4.973 | 70.702 | 4.19 [1.701-10.31] |
| 5.005 | 72.702 | 4.44 [1.628-12.111] |
| 5.019 | 73.550 | 7.42 [2.696-20.415] |
| 5.089 | 78.092 | 7.35 [2.306-23.435] |
| 5.408 | 102.698 | 5.55 [1.359-22.693] |
| 5.456 | 106.995 | 10.28 [2.509-42.081] |
| 5.534 | 114.388 | 18.3 [2.51-133.473] |
| 5.800 | 143.552 | |

### Example 14: Validation Study

In order to assess the risk of prostate cancer disease progression among men on active surveillance for prostate cancer, an independent validation study was conducted using a cohort of 248 participants (35 progressors, 213 non progressors). The levels of (TrihexosylCer(34:1), LactosylCer(36:0), LactosylCer(32:0), SM(44:2), SM(40:2), the plasma sphingolipid signature (SphingoSignature), and a simplified sphingolipid signature [Simplified Signature, consisting of TrihexosylCer(34:1), LactosylCer(36:0), and SM(40:2)] were tested in each participant. Cav-1 was not included in this analysis. The model was derived using fixed coefficients from the logistic regression as previously described.

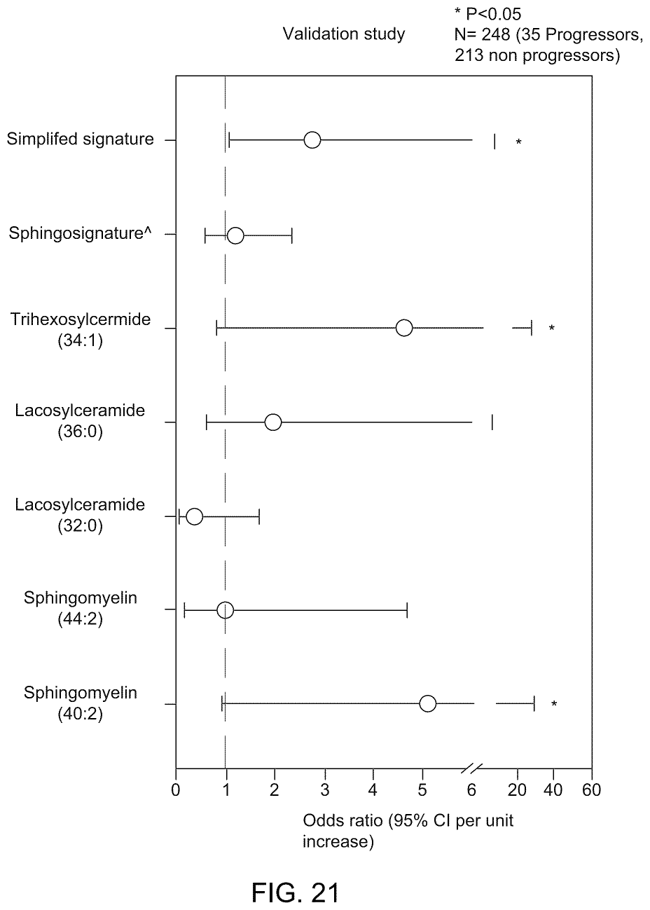

TrihexosylCer(34:1) and SM(40:2), along with the simplified sphingolipid signature, were found to have statistically significant odds ratios per unit increase, while hexosylceramide(40:0) was not quantifiable in the cohort samples (see FIG. 21).

### Other Embodiments

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

1.-3. (canceled)

4. A method of:
classifying a subject with prostate cancer as being at risk of developing aggressive prostate cancer or not being at risk of developing aggressive prostate cancer,
predicting a predisposition to aggressive prostate cancer in a subject,
diagnosing aggressive prostate cancer in a subject with prostate cancer,
determining the risk of a subject for having aggressive prostate cancer,
predicting the likelihood of progression of prostate cancer in a subject with prostate cancer,
providing a prognosis for a subject with prostate cancer, or
selecting a subject with prostate cancer for treatment with an anticancer therapy,
comprising:
(a) measuring the levels of trihexosylceramide 34:1 (TriHexCer 34:1) in a biological sample from said subject using an in vitro assay and
(b) comparing the levels of TriHexCer 34:1 in said sample with a reference,
wherein an altered amount of TriHexCer 34:1 relative to said reference provides an indication selected from the group consisting of:
an indication that the subject is at risk of developing aggressive prostate cancer or not at risk of developing aggressive prostate cancer,
an indication of a predisposition of the subject to aggressive prostate cancer,
an indication of the likelihood of progression of the prostate cancer in the subject,
an indication of progression-free survival of the subject,
an indication of the likely outcome of treatment of the prostate cancer, and
an indication that the subject is a candidate for treatment with an anticancer therapy.

5. A method of:
classifying a subject with prostate cancer as being at risk of developing aggressive prostate cancer or not being at risk of developing aggressive prostate cancer,
predicting a predisposition to aggressive prostate cancer in a subject,
diagnosing aggressive prostate cancer in a subject with prostate cancer,
determining the risk of a subject for having aggressive prostate cancer,
predicting the likelihood of progression of prostate cancer in a subject with prostate cancer,
providing a prognosis for a subject with prostate cancer, or
selecting a subject with prostate cancer for treatment with an anticancer therapy,
comprising:
(a) measuring the levels of sphingomyelin 40:2 (SM 40:2) in a biological sample from said subject using an in vitro assay and
(b) comparing the levels of SM 40:2 in said sample with a reference,
wherein an altered amount of SM 40:2 relative to said reference provides an indication selected from the group consisting of:
an indication that the subject is at risk of developing aggressive prostate cancer or not at risk of developing aggressive prostate cancer,
an indication of a predisposition of the subject to aggressive prostate cancer,
an indication of the likelihood of progression of the prostate cancer in the subject,
an indication of progression-free survival of the subject,
an indication of the likely outcome of treatment of the prostate cancer, and
an indication that the subject is a candidate for treatment with an anticancer therapy.

6.-49. (canceled)

50. The method claim 4, further comprising:
(a) measuring the levels of:
sphingomyelin 40:2 (SM 40:2), lactosylceramide 36:0 (LacCer 36:0), and TriHexCer 34:1 in a biological sample from said subject using an in vitro assay and
(b) comparing the levels of SM 40:2, LacCer 36:0, and TriHexCer 34:1 in said sample with a reference,
wherein an altered amount of SM 40:2, LacCer 36:0, and TriHexCer 34:1 relative to said reference provides an indication selected from the group consisting of:
an indication that the subject is at risk of developing aggressive prostate cancer or not at risk of developing aggressive prostate cancer,
an indication of a predisposition of the subject to aggressive prostate cancer,
an indication of the likelihood of progression of the prostate cancer in the subject,
an indication of progression-free survival of the subject,
an indication of the likely outcome of treatment of the prostate cancer, and
an indication that the subject is a candidate for treatment with an anticancer therapy.

51. The method claim 4, further comprising:
(a) measuring the levels of:
Caveolin-1 (CAV-1), sphingomyelin 40:2 (SM 40:2), sphingomyelin 44:2 (SM 44:2), lactosylceramide 32:0 (LacCer 32:0), lactosylceramide 36:0 (LacCer 36:0), trihexosylceramide 34:1 (TriHexCer 34:1), and hexosylceramide 40:0 (HexCer 40:0) in a biological sample from said subject using an in vitro assay and
(b) comparing the levels of CAV-1, SM 40:2, SM 44:2, LacCer 32:0, LacCer 36:0, TriHexCer 34:1, and HexCer 40:0 in said sample with a reference,
wherein an altered amount of CAV-1, SM 40:2, SM 44:2, LacCer 32:0, LacCer 36:0, TriHexCer 34:1, and HexCer 40:0 relative to said reference provides an indication selected from the group consisting of:
an indication that the subject is at risk of developing aggressive prostate cancer or not at risk of developing aggressive prostate cancer,
an indication of a predisposition of the subject to aggressive prostate cancer,
an indication of the likelihood of progression of the prostate cancer in the subject,
an indication of progression-free survival of the subject,
an indication of the likely outcome of treatment of the prostate cancer, and
an indication that the subject is a candidate for treatment with an anticancer therapy.

52. The method claim 4, further comprising:
(a) measuring the levels of:
sphingomyelin 40:2 (SM 40:2), sphingomyelin 44:2 (SM 44:2), lactosylceramide 32:0 (LacCer 32:0), lactosylceramide 36:0 (LacCer 36:0), and trihexosylceramide 34:1 (TriHexCer 34:1) in a biological sample from said subject using an in vitro assay and
(b) comparing the levels of SM 40:2, SM 44:2, LacCer 32:0, LacCer 36:0, and TriHexCer 34:1 in said sample with a reference,
wherein an altered amount of SM 40:2, SM 44:2, LacCer 32:0, LacCer 36:0, and TriHexCer 34:1 relative to said reference provides an indication selected from the group consisting of:
an indication that the subject is at risk of developing aggressive prostate cancer or not at risk of developing aggressive prostate cancer,
an indication of a predisposition of the subject to aggressive prostate cancer,
an indication of the likelihood of progression of the prostate cancer in the subject,
an indication of progression-free survival of the subject,
an indication of the likely outcome of treatment of the prostate cancer, and
an indication that the subject is a candidate for treatment with an anticancer therapy.

53. The method of claim 4, wherein the subject has prostate cancer.

54. The method of claim 51, wherein the levels of CAV-1, SM 40:2, SM 44:2, LacCer 32:0, LacCer 36:0, TriHexCer 34:1, and/or HexCer 40:0 are elevated in the subject relative to a healthy subject.

55. The method of claim 51, wherein the measuring of the CAV-1, SM 40:2, SM 44:2, LacCer 32:0, LacCer 36:0, TriHexCer 34:1, and/or HexCer 40:0 is carried out by UV-visible spectroscopy, mass spectrometry, nuclear magnetic resonance (NMR) spectroscopy, proton NMR spectroscopy, nuclear magnetic resonance (NMR) spectrometry, gas chromatography-mass spectrometry (GC-MS), liquid chromatography-mass spectrometry (LC-MS), high performance liquid chromatography (HPLC), ultra performance liquid chromatography (UPLC), liquid chromatography-mass spectrometry (LC-MS), correlation spectroscopy (COSy), nuclear Overhauser effect spectroscopy (NOESY), rotating frame nuclear Overhauser effect spectroscopy (ROESY), LC-TOF-MS, LC-MS/MS, or capillary electrophoresis-mass spectrometry.

56. A diagnostic panel for aggressive prostate cancer comprising trihexosylceramide 34:1 (TriHexCer 34:1) and/or sphingomyelin 40:2 (SM 40:2).

57. The diagnostic panel of claim 56, further comprising Caveolin-1 (CAV-1), sphingomyelin 40:2 (SM 40:2), sphingomyelin 44:2 (SM 44:2), lactosylceramide 32:0 (LacCer 32:0), lactosylceramide 36:0 (LacCer 36:0), trihexosylceramide 34:1 (TriHexCer 34:1) and hexosylceramide 40:0 (HexCer 40:0).

58. The diagnostic panel of claim 56, further comprising sphingomyelin 40:2 (SM 40:2), sphingomyelin 44:2 (SM 44:2), lactosylceramide 32:0 (LacCer 32:0), lactosylceramide 36:0 (LacCer 36:0), and trihexosylceramide 34:1 (TriHexCer 34:1).

59. The diagnostic panel of claim 56, further comprising sphingomyelin 40:2 (SM 40:2), lactosylceramide 36:0 (LacCer 36:0), and trihexosylceramide 34:1 (TriHexCer 34:1).

60. The diagnostic panel of claim 56, further comprising sphingomyelin 40:2 (SM 40:2), lactosylceramide 36:0 (LacCer 36:0), and trihexosylceramide 34:1.

61. A method of treatment or prevention of progression of prostate cancer in a subject in whom the levels of trihexosylceramide 34:1 and/or sphingomyelin 40:2 (SM 40:2) are elevated relative to a reference without prostate cancer, comprising one or more of:
administering an anticancer drug to the subject with prostate cancer;
administering therapeutic radiation to the subject with prostate cancer; and
surgery for partial or complete surgical removal of cancerous tissue in the subject with prostate cancer.

62. The method of claim 61, wherein the levels of Caveolin-1 (CAV-1), sphingomyelin 40:2 (SM 40:2), sphingomyelin 44:2 (SM 44:2), lactosylceramide 32:0 (LacCer 32:0), lactosylceramide 36:0 (LacCer 36:0), trihexosylceramide 34:1 and hexosylceramide 40:0 are elevated relative to a reference without prostate cancer.

63. The method of claim 61, wherein the levels of sphingomyelin 40:2 (SM 40:2), sphingomyelin 44:2 (SM 44:2), lactosylceramide 32:0 (LacCer 32:0), lactosylceramide 36:0 (LacCer 36:0), and trihexosylceramide 34:1 are elevated relative to a reference without prostate cancer.

64. The method of claim 61, wherein the levels of sphingomyelin 40:2 (SM 40:2), lactosylceramide 36:0 (LacCer 36:0), and trihexosylceramide 34:1 are elevated relative to a reference without prostate cancer.

65. The method of claim 61, wherein the levels of sphingomyelin 40:2 (SM 40:2), lactosylceramide 36:0 (LacCer 36:0), and trihexosylceramide 34:1 are elevated relative to a reference without prostate cancer.

66. The method as recited in claim 61 comprising, as a prior step,
classifying a subject with prostate cancer as being at risk of developing aggressive prostate cancer or not being at risk of developing aggressive prostate cancer,
predicting a predisposition to aggressive prostate cancer in a subject,
diagnosing aggressive prostate cancer in a subject with prostate cancer,
determining the risk of a subject for having aggressive prostate cancer,
predicting the likelihood of progression of prostate cancer in a subject with prostate cancer,
providing a prognosis for a subject with prostate cancer, or
selecting a subject with prostate cancer for treatment with an anticancer therapy.
